(19) United States
(12) Patent Application Publication
(10) Pub. No.: US 2025/0195617 A1
(43) Pub. Date: Jun. 19, 2025

(54) Liquid Pharmaceutical Formulations of CNP Compounds

(71) Applicant: Ascendis Pharma Growth Disorders A/S, Hellerup (DK)

(72) Inventors: Ulrich HERSEL, Heidelberg (DE); Tom WOODS, Heidelberg (DE); Andrea LADIGES, Heidelberg (DE); Steffen SCHRÖDER, Heidelberg (DE)

(21) Appl. No.: 18/867,010

(22) PCT Filed: May 22, 2023
(86) PCT No.: PCT/EP2023/063596

§ 371 Date: Nov. 19, 2024

(30) Foreign Application Priority Data
May 23, 2022 (EP) 22174758.7

(51) Int. Cl. A61K 38/22; A61K 9/08; A61K 47/10; A61K 47/26; A61K 47/60

(52) U.S. Cl. CPC A61K 38/2242; A61K 9/08; A61K 47/10; A61K 47/26; A61K 47/60

(57) ABSTRACT

A liquid pharmaceutical formulation comprising a CNP compound, a buffering agent, an isotonicity agent, a preservative and optionally an antioxidant and methods for making and using such formulation.

The present invention relates to liquid pharmaceutical formulations comprising a CNP compound, a buffering agent, an isotonicity agent, a preservative and optionally an antioxidant and to methods for making and using such formulations.

Achondroplasia (ACH) is a genetic disorder which occurs due to an autosomal dominant mutation in the fibroblast-growth-factor-receptor 3 (FGFR3) gene, that causes an abnormality of cartilage formation and results in dwarfism. C-type Natriuretic Peptide (CNP) is a hormone that binds and activates the peptide receptor B (NPR-B) resulting in the inhibition of FGFR3 downstream signaling. This in turn triggers endochondral growth and skeletal overgrowth, as observed in both mice and humans overexpressing CNP. Overproduction of CNP in the cartilage or continuous delivery of CNP through intravenous (iv) infusion normalizes the dwarfism of achondroplasic mice, suggesting that administration of CNP at supraphysiological levels is a strategy for treating ACH.

The human prepro-CNP which comprises 126 amino acids is further cleaved by furin to yield CNP-53. The CNP-53 possesses biological activity but is typically processed by an unknown mechanism to the biologically active 22 amino acids form in circulation, i.e. CNP-22. The bioactivity of CNP is tightly regulated and its clearance from the plasma is very rapid. Therefore, given the short in vivo half-life of CNP (2 min after intravenous administration), its use as a therapeutic agent is challenging in a pediatric population because it would require continuous infusion.

Various approaches for increasing the in vivo half-life of CNP were explored. For instance, Lorget et al. (Am. J. Hum. Genet. 91, 1108-1114, 2012) discloses a recombinant CNP-39 (also known as BMN111), consisting of the 37 C-terminal amino acids of human CNP-53 plus glycine and proline added at the N-terminus of the peptide, that mimics the pharmacological activity of human CNP.

An expansion of the scope of increasing the in vivo half-life of CNP based on conjugation to water-soluble carrier moieties such as PEG, via reversible prodrug linkers was explored in WO 2016/110577 A1, WO 2017/118693 A1, WO 2017/118698 A1, WO 2017/118700 A1, WO 2017/118703 A1, WO 2017/118704 A1 and WO 2017/118707 A1. Pharmaceutical formulations of such reversible CNP conjugates, wherein the carrier is attached to CNP via a reversible linkage, have to provide sufficient stability of the CNP conjugate in order to avoid premature CNP release during storage. In case the reversible linkage between the carrier and CNP is degraded during storage, the concentration of the readily available drug is increased, and this could lead to risk of overdosage upon administration, which may result in hypotension. In addition, any drug released during storage is subject to rapid renal clearance upon application to a patient, and consequently the time for which the long-acting composition provides therapeutically relevant amounts of drug is reduced. This poses a risk of unmet medical needs.

As degradation products that may form during storage may impair the bioactivity of the CNP moiety, it is thus desirable to minimize their formation. Moreover, within the reversible CNP conjugates the reversible linkage between the CNP moiety and the carrier moiety makes the storage of the pharmaceutical formulation comprising the CNP conjugates challenging.

WO 2020/165081 A1 teaches dry pharmaceutical formulations comprising a reversible CNP conjugate which requires reconstitution in an aqueous solution to provide an injectable pharmaceutical.

It is also important to identify suitable liquid pharmaceutical formulations of CNP compounds, particularly of reversible CNP conjugates comprising CNP covalently linked via a reversible linker to a carrier moiety, wherein the peptide will exhibit an acceptable impurity profile. Also, particularly for reversible CNP conjugates it is important to identify suitable liquid formulations displaying limited premature CNP release even after extended storage.

Thus, there remains a need in the art for ready-to-use liquid formulations of CNP compounds, particularly of reversible CNP conjugates, that exhibit storage stability and provide ease of use.

It is thus an object of the present invention to at least partially overcome the shortcomings described above.

This object is achieved with a liquid pharmaceutical formulation comprising a CNP compound, a buffering agent, an isotonicity agent, a preservative and optionally an antioxidant.

It was surprisingly found that the liquid pharmaceutical formulation of the present invention allows for stable long-term storage. The liquid pharmaceutical formulation of the present invention exhibits a substantial degree of peptide stability after storage for several months and without the need of freezing it, such as without the need of storing it in a freezer.

Within the meaning of the present invention the terms are used as follows.

As used herein, the term “about” in combination with a numerical value is used to indicate a range ranging from and including the numerical value plus and minus no more than 20% of said numerical value, in certain embodiments, no more than 10% of said numerical value, in certain embodiments, no more than 8% of said numerical value, in certain embodiments, no more than 5% of said numerical value and in certain embodiments, no more than 2% of said numerical value. For example, the phrase “about 200” is used to mean a range ranging from and including 200+/−10%, i.e. ranging from and including 180 to 220; in certain embodiments, 200+/−8%, i.e. ranging from and including 184 to 216; in certain embodiments, ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “antimicrobial” refers to a chemical substance, such as a chemical substance that kills or inhibits the growth of microorganisms, such as bacteria, fungi, yeasts, protozoans, molds and/or destroys viruses.

As used herein, the term “anti-adsorption agents” refers to mainly ionic or non-ionic surfactants, proteins or soluble polymers used to coat or adsorb competitively to the inner surface of the container comprising the formulation. Chosen concentration and type of excipient depends on the effect to be avoided but typically a monolayer of surfactant is formed at the interface just above the critical micelle concentration (CMC).

As used herein, the term “buffer” or “buffering agent” refers to a chemical compound that maintains the pH in a desired range. Physiologically tolerated buffers are, for example, sodium phosphate, histidine, bicarbonate, citrate, acetate, sulfate, nitrate, chloride and pyruvate. Antacids such as Mg(OH)₂ or ZnCO₃ may be also used.

As used herein, the term “CNP” refers to all CNP polypeptides, in certain embodiments from mammalian species, such as from human and mammalian species, in particular from human and murine species, as well as their variants, analogs, orthologs, homologs and derivatives and fragments thereof, that are characterized by regulating the growth, proliferation and differentiation of cartilaginous growth plate chondrocytes. The term “CNP” also includes all CNP variants, analogs, orthologs, homologs, derivatives and fragments thereof. The CNP variants, analogs, orthologs, homologs, derivatives and fragments thereof as disclosed in WO 2009/067639 A2 and WO 2010/135541 A2 are herewith incorporated by reference.

As used herein, the term “CNP polypeptide variant” refers to a polypeptide from the same species that differs from a reference CNP polypeptide. Generally, differences are limited so that the amino acid sequence of the reference and the variant are closely similar overall and, in many regions, identical. In certain embodiments, CNP polypeptide variants are at least 70%, 80%, 90%, or 95% identical to a reference CNP polypeptide. By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. These alterations of the reference sequence may occur at the amino (N-terminal) or carboxy terminal (C-terminal) positions of the reference amino acid sequence or anywhere between those terminal positions, interspersed either individually among residues in the reference sequence or in one or more contiguous groups within the reference sequence. The query sequence may be an entire amino acid sequence of the reference sequence or any fragment specified as described herein. Such CNP polypeptide variants may be naturally occurring variants, such as naturally occurring allelic variants encoded by one of several alternate forms of a CNP occupying a given locus on a chromosome or an organism, or isoforms encoded by naturally occurring splice variants originating from a single primary transcript. Alternatively, a CNP polypeptide variant may be a variant that is not known to occur naturally and that can be made by mutagenesis techniques known in the art. It is known in the art that one or more amino acids may be deleted from the N-terminus or C-terminus of a bioactive peptide or protein without substantial loss of biological function. Such N- and/or C-terminal deletions are also encompassed by the term CNP polypeptide variant.

It is also recognized by one of ordinary skill in the art that some amino acid sequences of CNP polypeptides can be varied without significant effect on the structure or function of the peptide. Such mutants include deletions, insertions, inversions, repeats, and substitutions selected according to general rules known in the art so as to have little effect on activity. For example, guidance concerning how to make phenotypically silent amino acid substitutions is provided in Bowie et al. (1990), Science 247:1306-1310, which is hereby incorporated by reference in its entirety, wherein the authors indicate that there are two main approaches for studying the tolerance of the amino acid sequence to change.

As used herein, the term “CNP analog” refers to CNP of different and unrelated organisms which perform the same functions in each organism, but which did not originate from an ancestral structure that the organisms' ancestors had in common. Instead, analogous CNPs arose separately and then later evolved to perform the same or similar functions. In other words, analogous CNP polypeptides are polypeptides with quite different amino acid sequences that perform the same biological activity, namely regulating the growth, proliferation and differentiation of cartilaginous growth plate chondrocytes.

As used herein, the term “CNP ortholog” refers to CNP within two different species whose sequences are related to each other via a common homologous CNP in an ancestral species, but which have evolved to become different from each other.

As used herein, the term “CNP homolog” refers to CNP of different organisms which perform the same functions in each organism and which originate from an ancestral structure that the organisms' ancestors had in common. In other words, homologous CNP polypeptides are polypeptides with quite similar amino acid sequences that perform the same biological activity, namely regulating the growth, proliferation and differentiation of cartilaginous growth plate chondrocytes. In certain embodiments, CNP polypeptide homologs may be defined as polypeptides exhibiting at least 40%, 50%, 60%, 70%, 80%, 90% or 95% identity to a reference CNP polypeptide.

Thus, a CNP polypeptide may be, for example: (i) one in which at least one of the amino acid residues is substituted with a conserved or non-conserved amino acid residue, in certain embodiments, a conserved amino acid residue, and such substituted amino acid residue may or may not be one encoded by the genetic code; and/or (ii) one in which at least one of the amino acid residues includes a substituent group; and/or (iii) one in which the CNP polypeptide is fused with another compound, such as a compound to increase the half-life of the polypeptide (for example, polyethylene glycol); and/or (iv) one in which additional amino acids are fused to the CNP polypeptide, such as an IgG Fc fusion region peptide or leader or secretory sequence or a sequence which is employed for purification of the above form of the polypeptide or a pre-protein sequence.

As used herein, the term “CNP polypeptide fragment” refers to any peptide comprising a continuous span of a part of the amino acid sequence of a CNP polypeptide.

More specifically, a CNP polypeptide fragment comprises at least 6, such as at least 8, at least 10 or at least 17 consecutive amino acids of a CNP polypeptide. A CNP polypeptide fragment may additionally be described as sub-genuses of CNP polypeptides comprising at least 6 amino acids, wherein “at least 6” is defined as any integer between 6 and the integer representing the C-terminal amino acid of a CNP polypeptide. Further included are species of CNP polypeptide fragments at least 6 amino acids in length, as described above, that are further specified in terms of their N-terminal and C-terminal positions. Also encompassed by the term “CNP polypeptide fragment” as individual species are all CNP polypeptide fragments, at least 6 amino acids in length, as described above, that may be particularly specified by a N-terminal and C-terminal position. That is, every combination of an N-terminal and C-terminal position that a fragment of at least 6 contiguous amino acid residues in length could occupy, on any given amino acid sequence of a CNP polypeptide.

As the term CNP includes the above-described variants, analogs, orthologs, homologs, derivatives and fragments of CNP, all references to specific positions within a reference sequence also include the equivalent positions in the variants, analogs, orthologs, homologs, derivatives and fragments of a CNP moiety, even if not explicitly mentioned.

Naturally occurring CNP-22 (SEQ ID NO:1) has the following sequence:

GLSKGCFGLKLDRIGSMSGLGC,

wherein the cysteines at position 6 and 22 are connected through a disulfide-bridge.

In certain embodiments, the term “CNP” also refers to the following peptide sequences:

SEQ ID NO: 2 (CNP-53):
DLRVDTKSRAAWARLLQEHPNARKYKGANKKGLSKGCFGLKLDRIGSM
SGLGC;
SEQ ID NO: 3 (G-CNP-53):
GDLRVDTKSRAAWARLLQEHPNARKYKGANKKGLSKGCFGLKLDRIGS
MSGLGC;
SEQ ID NO: 4 (M-CNP-53):
MDLRVDTKSRAAWARLLQEHPNARKYKGANKKGLSKGCFGLKLDRIGS
MSGLGC;
SEQ ID NO: 5 (P-CNP-53):
PDLRVDTKSRAAWARLLQEHPNARKYKGANKKGLSKGCFGLKLDRIGS
MSGLGC;
SEQ ID NO: 6 (CNP-53 M48N):
DLRVDTKSRAAWARLLQEHPNARKYKGANKKGLSKGCFGLKLDRIGSN
SGLGC;
SEQ ID NO: 7 (CNP-53 Δ15-31):
DLRVDTKSRAAWARGLSKGCFGLKLDRIGSMSGLGC;
SEQ ID NO: 8 (CNP-52):
LRVDTKSRAAWARLLQEHPNARKYKGANKKGLSKGCFGLKLDRIGSMS
GLGC;
SEQ ID NO: 9 (CNP-51):
RVDTKSRAAWARLLQEHPNARKYKGANKKGLSKGCFGLKLDRIGSMSG
LGC;
SEQ ID NO: 10 (CNP-50):
VDTKSRAAWARLLQEHPNARKYKGANKKGLSKGCFGLKLDRIGSMSGL
GC;
SEQ ID NO: 11 (CNP-49):
DTKSRAAWARLLQEHPNARKYKGANKKGLSKGCFGLKLDRIGSMSGLG
C;
SEQ ID NO: 12 (CNP-48):
TKSRAAWARLLQEHPNARKYKGANKKGLSKGCFGLKLDRIGSMSGLGC;
SEQ ID NO: 13 (CNP-47):
KSRAAWARLLQEHPNARKYKGANKKGLSKGCFGLKLDRIGSMSGLGC;
SEQ ID NO: 14 (CNP-46):
SRAAWARLLQEHPNARKYKGANKKGLSKGCFGLKLDRIGSMSGLGC;
SEQ ID NO: 15 (CNP-45):
RAAWARLLQEHPNARKYKGANKKGLSKGCFGLKLDRIGSMSGLGC;
SEQ ID NO: 16 (CNP-44):
AAWARLLQEHPNARKYKGANKKGLSKGCFGLKLDRIGSMSGLGC;
SEQ ID NO: 17 (CNP-44 Δ14-22):
AAWARLLQEHPNAGLSKGCFGLKLDRIGSMSGLGC;
SEQ ID NO: 18 (CNP-44 Δ15-22):
AAWARLLQEHPNARGLSKGCFGLKLDRIGSMSGLGC;
SEQ ID NO: 19 (CNP-43):
AWARLLQEHPNARKYKGANKKGLSKGCFGLKLDRIGSMSGLGC;
SEQ ID NO: 20 (CNP-42):
WARLLQEHPNARKYKGANKKGLSKGCFGLKLDRIGSMSGLGC;
SEQ ID NO: 21 (CNP-41):
ARLLQEHPNARKYKGANKKGLSKGCFGLKLDRIGSMSGLGC;
SEQ ID NO: 22 (CNP-40):
RLLQEHPNARKYKGANKKGLSKGCFGLKLDRIGSMSGLGC;
SEQ ID NO: 23 (CNP-39):
LLQEHPNARKYKGANKKGLSKGCFGLKLDRIGSMSGLGC;
SEQ ID NO: 24 (CNP-38):
LQEHPNARKYKGANKKGLSKGCFGLKLDRIGSMSGLGC,

wherein the cysteines at position 22 and 38 are connected through a disulfide-bridge;

SEQ ID NO: 25 (CNP-37):
QEHPNARKYKGANKKGLSKGCFGLKLDRIGSMSGLGC;
SEQ ID NO: 26 (CNP-37 Q1pQ, wherein pQ =
pyroglutamate):
pQEHPNARKYKGANKKGLSKGCFGLKLDRIGSMSGLGC;
SEQ ID NO: 27 (G-CNP-37):
GQEHPNARKYKGANKKGLSKGCFGLKLDRIGSMSGLGC;
SEQ ID NO: 28 (P-CNP-37):
PQEHPNARKYKGANKKGLSKGCFGLKLDRIGSMSGLGC;
SEQ ID NO: 29 (M-CNP-37):
MQEHPNARKYKGANKKGLSKGCFGLKLDRIGSMSGLGC;
SEQ ID NO: 30 (PG-CNP-37):
PGQEHPNARKYKGANKKGLSKGCFGLKLDRIGSMSGLGC;
SEQ ID NO: 31 (MG-CNP-37):
MGQEHPNARKYKGANKKGLSKGCFGLKLDRIGSMSGLGC;
SEQ ID NO: 32 (CNP-37 M32N):
QEHPNARKYKGANKKGLSKGCFGLKLDRIGSNSGLGC;
SEQ ID NO: 33 (G-CNP-37 M32N):
GQEHPNARKYKGANKKGLSKGCFGLKLDRIGSNSGLGC;
SEQ ID NO: 34 (G-CNP-37 K14Q):
GQEHPNARKYKGANQKGLSKGCFGLKLDRIGSMSGLGC;
SEQ ID NO: 35 (G-CNP-37 K14P):
GQEHPNARKYKGANPKGLSKGCFGLKLDRIGSMSGLGC;
SEQ ID NO: 36 (G-CNP-37 K14Q, Δ15):
GQEHPNARKYKGANQGLSKGCFGLKLDRIGSMSGLGC;
SEQ ID NO: 37 (G-CNP-37 K14Q, K15Q):
GQEHPNARKYKGANQQGLSKGCFGLKLDRIGSMSGLGC;
SEQ ID NO: 38 (CNP-36):
EHPNARKYKGANKKGLSKGCFGLKLDRIGSMSGLGC;
SEQ ID NO: 39 (CNP-35):
HPNARKYKGANKKGLSKGCFGLKLDRIGSMSGLGC;
SEQ ID NO: 40 (CNP-34):
PNARKYKGANKKGLSKGCFGLKLDRIGSMSGLGC;
SEQ ID NO: 41 (CNP-33):
NARKYKGANKKGLSKGCFGLKLDRIGSMSGLGC;
SEQ ID NO: 42 (CNP-32):
ARKYKGANKKGLSKGCFGLKLDRIGSMSGLGC;
SEQ ID NO: 43 (CNP-31):
RKYKGANKKGLSKGCFGLKLDRIGSMSGLGC;
SEQ ID NO: 44 (CNP-30):
KYKGANKKGLSKGCFGLKLDRIGSMSGLGC;
SEQ ID NO: 45 (CNP-29):
YKGANKKGLSKGCFGLKLDRIGSMSGLGC;
SEQ ID NO: 46 (CNP-28):
KGANKKGLSKGCFGLKLDRIGSMSGLGC;
SEQ ID NO: 47 (GHKSEVAHRF-CNP-28):
GHKSEVAHRFKGANKKGLSKGCFGLKLDRIGSMSGLGC;
SEQ ID NO: 48 (CNP-27):
GANKKGLSKGCFGLKLDRIGSMSGLGC;
SEQ ID NO: 49 (CNP-27 K4Q, K5Q):
GANQQGLSKGCFGLKLDRIGSMSGLGC;
SEQ ID NO: 50 (CNP-27 K4R, K5R):
GANRRGLSKGCFGLKLDRIGSMSGLGC;
SEQ ID NO: 51 (CNP-27 K4P, K5R):
GANPRGLSKGCFGLKLDRIGSMSGLGC;
SEQ ID NO: 52 (CNP-27 K4S, K5S):
GANSSGLSKGCFGLKLDRIGSMSGLGC;
SEQ ID NO: 53 (CNP-27 K4P, K5R):
GANGANPRGLSRGCFGLKLDRIGSMSGLGC;
SEQ ID NO: 54 (CNP-27 K4R, K5R, K9R):
GANRRGLSRGCFGLKLDRIGSMSGLGC;
SEQ ID NO: 55 (CNP-27 K4R, K5R, K9R, M22N):
GANRRGLSRGCFGLKLDRIGSNSGLGC;
SEQ ID NO: 56 (P-CNP-27 K4R, K5R, K9R):
PGANRRGLSRGCFGLKLDRIGSMSGLGC;
SEQ ID NO: 57 (M-CNP-27 K4R, K5R, K9R):
MGANRRGLSRGCFGLKLDRIGSMSGLGC;
SEQ ID NO: 58 (HSA fragment-CNP-27):
GHKSEVAHRFKGANKKGLSKGCFGLKLDRIGSMSGLG;
SEQ ID NO: 59 (HSA fragment-CNP-27 M22N):
GHKSEVAHRFKGANKKGLSKGCFGLKLDRIGSNSGLGC;
SEQ ID NO: 60 (M-HSA fragment-CNP-27):
MGHKSEVAHRFKGANKKGLSKGCFGLKLDRIGSMSGLGC;
SEQ ID NO: 61 (P-HSA fragment-CNP-27):
PGHKSEVAHRFKGANKKGLSKGCFGLKLDRIGSMSGLGC;
SEQ ID NO: 62 (CNP-26):
ANKKGLSKGCFGLKLDRIGSMSGLGC;
SEQ ID NO: 63 (CNP-25):
NKKGLSKGCFGLKLDRIGSMSGLGC;
SEQ ID NO: 64 (CNP-24):
KKGLSKGCFGLKLDRIGSMSGLGC;
SEQ ID NO: 65 (CNP-23):
KGLSKGCFGLKLDRIGSMSGLGC;
SEQ ID NO: 66 (R-CNP-22):
RGLSKGCFGLKLDRIGSMSGLGC;
SEQ ID NO: 67 (ER-CNP-22):
ERGLSKGCFGLKLDRIGSMSGLGC;
SEQ ID NO: 68 (R-CNP-22 K4R):
RGLSRGCFGLKLDRIGSMSGLGC;
SEQ ID NO: 69 (ER-CNP-22 4KR):
ERGLSRGCFGLKLDRIGSMSGLGC;
SEQ ID NO: 70 (RR-CNP-22):
RRGLSRGCFGLKLDRIGSMSGLGC;
SEQ ID NO: 71 (HRGP fragment-CNP-22):
GHHSHEQHPHGANQQGLSKGCFGLKLDRIGSMSGLGC;
SEQ ID NO: 72 (HRGP fragment-CNP-22):
GAHHPHEHDTHGANQQGLSKGCFGLKLDRIGSMSGLGC;
SEQ ID NO: 73 (HRGP fragment-CNP-22):
GHHSHEQHPHGANPRGLSKGCFGLKLDRIGSMSGLGC;
SEQ ID NO: 74 (IgG₁(F_{c}) fragment-CNP-22):
GQPREPQVYTLPPSGLSKGCFGLKLDRIGSMSGLGC;
SEQ ID NO: 75 (HSA fragment-CNP-22):
GQHKDDNPNLPRGANPRGLSKGCFGLKLDRIGSMSGLGC;
SEQ ID NO: 76 (HSA fragment-CNP-22):
GERAFKAWAVARLSQGLSKGCFGLKLDRIGSMSGLGC;
SEQ ID NO: 77 (osteocrin NPR C inhibitor
fragment-CNP22):
FGIPMDRIGRNPRGLSKGCFGLKLDRIGSMSGLGC;
SEQ ID NO: 78 (FGF2 heparin-binding domain
fragment-CNP22):
GKRTGQYKLGSKTGPGPKGLSKGCFGLKLDRIGSMSGLGC;
SEQ ID NO: 79 (IgG₁(F_{c}) fragment-CNP-22 K4R):
GQPREPQVYTGANQQGLSRGCFGLKLDRIGSMSGLGC;
SEQ ID NO: 80 (HSA fragment-CNP-22 K4R):
GVPQVSTSTGANQQGLSRGCFGLKLDRIGSMSGLGC;
SEQ ID NO: 81 (fibronectin fragment-CNP-22 K4R):
GQPSSSSQSTGANQQGLSRGCFGLKLDRIGSMSGLGC;
SEQ ID NO: 82 (fibronectin fragment-CNP-22 K4R):
GQTHSSGTQSGANQQGLSRGCFGLKLDRIGSMSGLGC;
SEQ ID NO: 83 (fibronectin fragment-CNP-22 K4R):
GSTGQWHSESGANQQGLSRGCFGLKLDRIGSMSGLGC;
SEQ ID NO: 84 (zinc finger fragment-CNP-22 K4R):
GSSSSSSSSSGANQQGLSRGCFGLKLDRIGSMSGLGC;
SEQ ID NO: 85 (CNP-21):
LSKGCFGLKLDRIGSMSGLGC;
SEQ ID NO: 86 (CNP-20):
SKGCFGLKLDRIGSMSGLGC;
SEQ ID NO: 87 (CNP-19):
KGCFGLKLDRIGSMSGLGC;
SEQ ID NO: 88 (CNP-18):
GCFGLKLDRIGSMSGLGC;
SEQ ID NO: 89 (CNP-17):
CFGLKLDRIGSMSGLGC;
SEQ ID NO: 90 (BNP fragment-CNP-17-BNP fragment):
SPKMVQGSGCFGLKLDRIGSMSGLGCKVLRRH;
SEQ ID NO: 91 (CNP-38 L1G):
GQEHPNARKYKGANKKGLSKGCFGLKLDRIGSMSGLGC;
SEQ ID NO: 92 (Ac-CNP-37; wherein Ac = acetyl):
Ac-QEHPNARKYKGANKKGLSKGCFGLKLDRIGSMSGLGC;
SEQ ID NO: 93:
QEHPNARX₁YX₂GANX₃X₄GLSX₅GCFGLX₆LDRIGSMSGLGC,

wherein X₁, X₂, X₃, X₄, X₅ and X₆ are independently of each other selected from the group consisting of K, R, P, S and Q, with the provision that at least one of X₁, X₂, X₃, X₄, X₅ and X₆ is selected from the group consisting of R, P, S and Q; in certain embodiments, X₁, X₂, X₃, X₄, X₅ and X₆ are selected from the group consisting of K and R, with the provision that at least one of X₁, X₂, X₃, X₄, X₅ and X₆ is R;

SEQ ID NO: 94:
QEHPNARKYKGANX₁X₂GLSX₃GCFGLX₄LDRIGSMSGLGC,

wherein X₁, X₂, X₃ and X₄ are independently of each other selected from the group consisting of K, R, P, S and Q, with the provision that at least one of X₁, X₂, X₃ and X₄ is selected from the group consisting of R, P, S and Q; in certain embodiments, X₁, X₂, X₃ and X₄ are selected from K and R, with the provision that at least one of X₁, X₂, X₃ and X₄ is R;

SEQ ID NO: 95:
QEHPNARKYKGANX₁X₂GLSKGCFGLKLDRIGSMSGLGC,

wherein X₁X₂ are selected from the group consisting of KR, RK, KP, PK, SS, RS, SR, QK, QR, KQ, RQ, RR and QQ.

It is understood that also the equivalents of the cysteines in positions 22 and 38 of SEQ ID NO: 24 are connected through a disulfide-bridge in SEQ ID NOs: 2 to 95.

The term “CNP” also includes poly(amino acid) conjugates which have a sequence as described above, but have a backbone that comprises both amide and non-amide linkages, such as ester linkages, like for example depsipeptides. Depsipeptides are chains of amino acid residues in which the backbone comprises both amide (peptide) and ester bonds. Accordingly, the term “side chain” as used herein refers either to the moiety attached to the alpha-carbon of an amino acid moiety, if the amino acid moiety is connected through amine bonds such as in polypeptides, or to any carbon atom-comprising moiety attached to the backbone of a poly(amino acid) conjugate, such as for example in the case of depsipeptides. In certain embodiments, the term “CNP” refers to polypeptides having a backbone formed through amide (peptide) bonds.

As used herein, the term “ring moiety” refers to the stretch of consecutive amino acid residues of the CNP that is located between two cysteine residues that form an intramolecular disulfide bridge or between homologous amino acid residues which are connected through a chemical linker. Preferably, the ring moiety is located between two cysteine residues that form an intramolecular disulfide bridge. These two cysteines correspond to the cysteines at position 22 and position 38 in the sequence of CNP-38 (SEQ ID NO:24). Accordingly, amino acids 23 to 37 are located in said ring moiety, if the CNP has the sequence of CNP-38.

Independently of the length of the CNP, the sequence of the ring moiety of wild-type CNP is FGLKLDRIGSMSGLG (SEQ ID NO:96). In certain embodiments, the ring moiety of CNP is FGLKLDRIGSNSGLG (SEQ ID NO:97).

As the term CNP includes the above-described variants, analogs, orthologs, homologs, derivatives and fragments of CNP, the term “ring moiety” also includes the corresponding variants, analogs, orthologs, homologs, derivatives and fragments of the sequence of SEQ ID NO: 96. Accordingly, all references to specific positions within a reference sequence also include the equivalent positions in variants, analogs, orthologs, homologs, derivatives and fragments of a CNP moiety, even if not explicitly mentioned.

As used herein, the term “CNP agonist” refers to any molecule that activates the natriuretic peptide receptor B (NPR-B). In certain embodiments, said CNP agonist has an EC₅₀ that is at most 50-fold higher than the NPR-B activity of CNP-22 (SEQ ID NO:1).

As used herein, the term “CNP conjugate” refers to any compound, crystal or admixture that comprises at least one CNP moiety which is conjugated to at least one other moiety.

As used herein, the term “reversible CNP conjugate” refers to a CNP conjugate comprising at least one CNP moiety that is covalently and reversibly conjugated to a carrier moiety and from which at least one CNP moiety is released with a release half-life of at least 6 hours under physiological conditions.

As used herein, the term “stable CNP conjugate” refers to a CNP conjugate comprising at least one CNP moiety that is covalently and stably conjugated to a carrier moiety, i.e. via a stable linkage having a half-life under physiological conditions of more than six months.

As used herein, the phrase “CNP conjugate, of which CNP moiety” followed by an amount in mg/ml means that the liquid formulation comprises a CNP conjugate, but that for the corresponding amount only the CNP moiety is considered instead of taking the full CNP conjugate, i.e. moieties of the CNP conjugate other than the CNP moiety, such as the carrier moiety, are not taken into account. The amount of CNP moiety within a CNP conjugate can be determined by quantitative amino acid analysis or by any known analytical methods that allows quantification of the unknown sample compared to the CNP conjugate of a known CNP moiety content.

As used herein, the term “cryoprotectant” refers to a chemical compound that is added to a formulation in order to protect the CNP compound during freezing stages.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀, C₁₋₂₀ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH—CH₂, —CH—CH—CH₃, —CH₂—CH═CH₂, —CH═CH—CH₂—CH₃ and —CH—CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, —CH₂—CH₂—C≡CH and —CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which are in certain embodiments, selected from the group consisting of

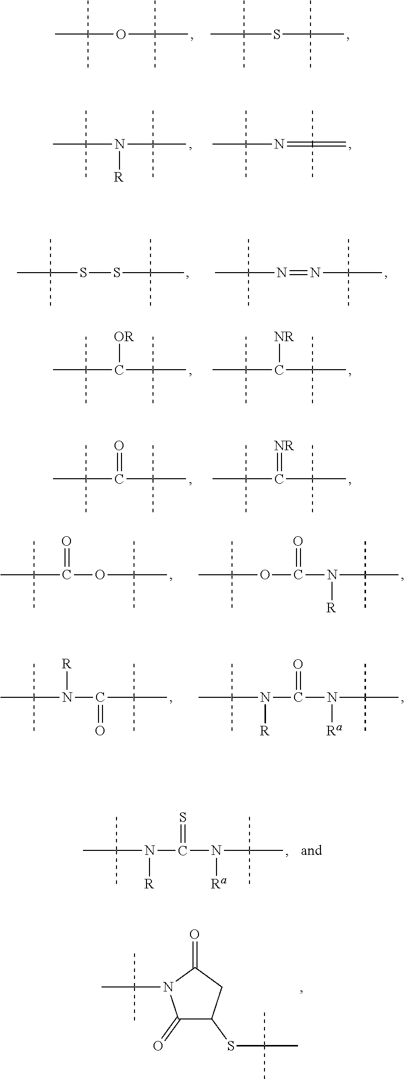

wherein
dashed lines indicate attachment to the remainder of the moiety or reagent; and —R and —R^{a} are independently of each other selected from the group consisting of —H, methyl, ethyl, propyl, butyl, pentyl and hexyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings, in certain embodiments of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including-S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair —R^{x}/—R^{y} is joined together with the atom to which they are attached to form a C₃₋₁₀ cycloalkyl or a 3- to 10-membered heterocyclyl” in relation with a moiety of the structure:

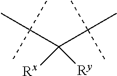

means that —R^{x} and —R^{y} form the following structure:

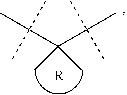

wherein R is C₃₋₁₀ cycloalkyl or 3- to 10-membered heterocyclyl.

It is also understood that the phrase “the pair —R^{x}/—R^{y} is joined together with the atoms to which they are attached to form a ring A” in relation with a moiety of the structure:

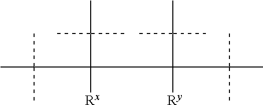

means that —R^{x} and —R^{y} form the following structure:

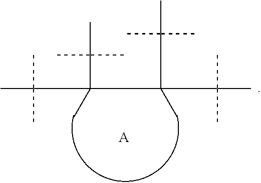

As used herein, the term “dry pharmaceutical formulation” means that a pharmaceutical formulation is provided which may be stored in a dry form such as for at least 1 week, such as for at least 2 weeks, such as for at least 1 month, such as for at least 6 months, such as for at least 12 months, such as for at least 18 months, such as for at least 24 months, such as for at least 36 months or such as for at least 48 months before administration. Suitable methods for drying are spray-drying and lyophilization, i.e. freeze-drying. Such dry pharmaceutical formulations comprising CNP compounds, particularly CNP conjugates have a residual water content of a maximum of 5%, in certain embodiments less than 2% and in certain embodiments less than 1%, as determined with Karl Fischer. In certain embodiments, the dry pharmaceutical formulation is dried by lyophilization.

As used herein, the term “drug” refers to a substance used in the treatment, cure, prevention, or diagnosis of a disease or used to otherwise enhance physical or mental well-being. If a drug, such as CNP, is conjugated to another moiety, the moiety of the resulting product that originated from the drug is referred to as “drug moiety”.

As used herein, the term “EC₅₀” with regards to a CNP agonist or CNP conjugate refers to the concentration of the CNP agonist or CNP conjugate with which a half-maximum cGMP production is elicited. NPR-B activity in the form of its EC₅₀ of the CNP agonist, of the CNP conjugate and of CNP-22 is measured by cultivating NIH-3T3 (Murine Embryo Fibroblast cell line) cells which express NPR-B on their cell surface, incubating the cells with the CNP agonist, CNP conjugate, the corresponding released CNP or CNP-22, respectively, and determining the intracellular production of the second messenger cGMP with a standard cGMP assay. In particular the assay is performed as follows:
(1) murine NIH-3T3 cells expressing endogenous NPR-B are cultivated in DMEM F-12 medium with 5% FBS and 5 mM glutamine at 37° C. and 5% CO₂;
(2) for each assay 50,000 cells are resuspended in Dulbecco's PBS with IBMX and incubated with either the CNP agonist, CNP conjugate, the corresponding released CNP or CNP-22; each in different concentrations;
(3) after incubating for 30 min at 37° C. and 5% CO₂, the cells are lysed and cGMP levels are determined; and
(4) generating an EC₅₀ value from the determined cGMP levels.

Preferably, the IBMX concentration in step (2) is 0.5 mM.

Step (3) can be performed using any assay for measuring cGMP which is a standard procedure well-known to the person skilled in the art. Preferably step (3) is done with a cGMP TR-FRET assay, more preferably with the cGMP TR-FRET assay from Cisbio, Cat. No. 62GM2PEB. As during such experiments the reversible CNP conjugate releases a certain amount of CNP which would distort the results, measurements for the NPR-B activity of the CNP conjugate are preferably made in the form of a stable CNP conjugate which does not release CNP.

As used herein, the term “excipient” refers to compounds administered together with the CNP compound, for example, buffering agents, isotonicity modifiers, preservatives, stabilizers, anti-adsorption agents, oxidation protection agents, or other auxiliary agents. However, in some cases, one excipient may have dual or triple functions. The term “excipient” may also refer to a diluent, adjuvant, or vehicle with which the CNP compound, is administered. Such pharmaceutical excipient can be sterile liquids, such as water and oils, including those of petroleum, animal, vegetable or synthetic origin, including, but not limited to peanut oil, soybean oil, mineral oil, sesame oil and the like. Water is a preferred excipient when the pharmaceutical formulation is administered orally. Saline and aqueous dextrose are preferred excipients when the pharmaceutical formulation is administered intravenously or subcutaneously. Saline solutions and aqueous dextrose and glycerol solutions are in certain embodiments, employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical formulation can also contain minor amounts of wetting or emulsifying agents, pH buffering agents, like, for example, acetate, succinate, Tris (tris(hydroxymethyl)aminomethane), carbonate, phosphate, HEPES (4-(2-hydroxyethyl)-1-piperazineethanesulfonic acid), MES (2-(N-morpholino)ethanesulfonic acid), or can contain detergents, like Tween®, poloxamers, poloxamines, CHAPS, Igepal®, or amino acids like, for example, glycine, lysine, or histidine. These pharmaceutical formulations can take the form of solutions, suspensions, emulsions, tablets, pills, capsules, powders, sustained-release formulations and the like. The pharmaceutical formulation can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such formulations will contain a therapeutically effective amount of the CNP compound, together with a suitable amount of excipient so as to provide the form for proper administration to the patient. The formulation should suit the mode of administration.

As used herein, the term “formulation” or “pharmaceutical formulation” refers to a formulation containing one or more CNP compounds and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, the pharmaceutical formulations of the present invention encompass any formulation or composition made by admixing one or more CNP compounds and a pharmaceutically acceptable excipient such as a buffering agent and isotonicity agent.

As used herein, the term “free form” of a drug refers to the drug in its unmodified, pharmacologically fully active form, e.g. after being released from the reversible CNP conjugate.

As used herein, the term “functional group” means a group of atoms which can react with other groups of atoms. Functional groups include, but are not limited, to the following groups: carboxylic acid (—(C═O) OH), primary or secondary amine (—NH₂, —NH—), maleimide, thiol (—SH), sulfonic acid (—(O═S═O) OH), carbonate, carbamate (—O(C═O)N<), hydroxyl (—OH), aldehyde (—(C═O)H), ketone (—(C—O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. It is generally preferred that halogen is fluoro or chloro.

As used herein, the term “interrupted” means that a moiety is inserted in between two carbon atoms or—if the insertion is at one of the ends of the moiety—between a carbon or heteroatom and a hydrogen atom, in certain embodiments between a carbon and a hydrogen atom.

As used herein, the term “isotonicity agent” refers to a compound that minimizes pain, irritation and tissue damage that can result from cell damage due to osmotic pressure differences between the injected solution and plasma.

As used herein, the term “liquid pharmaceutical formulation” means that a pharmaceutical formulation is provided and may be stored in a liquid form such as for at least 1 week, such as for at least 2 weeks, such as for at least 1 month, such as for at least 6 months, such as for at least 12 months, such as for at least 18 months, such as for at least 24 months, such as for at least 36 months or such as for at least 48 months before administration.

As used therein, the term “lyophilized formulation” means that the formulation comprising the CNP compound was first frozen and subsequently subjected to water removal by means of reduced pressure. This terminology does not exclude additional drying steps which occur in the manufacturing process prior to filling the formulation into the final container.

As used herein, the terms “lyophilization” or “freeze-drying” are interchangeable, and refer to a dehydration process, characterized by freezing a formulation and then reducing the surrounding pressure and, optionally, adding heat to allow the frozen water in the formulation to sublime directly from the solid phase to gas. Typically, the sublimed water is collected by desublimation.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety. Accordingly, a drug moiety, such as a CNP moiety, is released from a reversible CNP conjugate as a drug, such as CNP.

It is understood that if the sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R¹)—” can be attached to two moieties or interrupting a moiety either as “—C(O)N(R¹)—” or as “—N(R¹)C(O)—”. Similarly, a moiety:

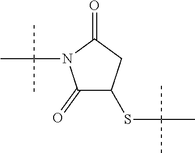

can be attached to two moieties or can interrupt a moiety either as:

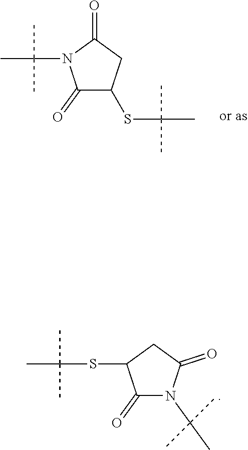

In case the CNP moiety comprises one or more acidic or basic groups, the liquid pharmaceutical formulation comprises also their corresponding pharmaceutically or toxicologically acceptable salts, in particular their pharmaceutically utilizable salts. Thus, the CNP moieties comprising one or more acidic groups can be present and used, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, and other salts or amines known to the person skilled in the art. CNP moieties comprising one or more basic groups, i.e. groups which can be protonated, can be present and can be used in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively charged ammonium group and an appropriate counterion of the salt. If the CNP moieties simultaneously comprise acidic and basic groups, the pharmaceutical formulations according to the present invention also include, in addition to the salt forms mentioned, inner salts or betaines (zwitterions). The respective salts can be obtained by customary methods which are known to the person skilled in the art like, for example by contacting these conjugates with an organic or inorganic acid or base in a solvent or dispersant, or by anion exchange or cation exchange with other salts.

As used herein, the term “natural product” refers to purified organic compounds isolated from natural sources that are produced by the pathways of primary or secondary metabolism.

As used herein, the term “oligonucleotide” refers to double- or single-stranded RNA and DNA with preferably 2 to 1000 nucleotides and any modifications thereof. Modifications include for example, those which provide other chemical groups that incorporate additional charge, polarizability, hydrogen bonding, electrostatic interaction, and fluxionality to the nucleic acid ligand bases or to the nucleic acid ligand as a whole. Such modifications include for example, to 2′-position sugar modifications, 5-position pyrimidine modifications, 8-position purine modifications, modifications at exocyclic amines, substitution of 4-thiouridines, substitution of 5-bromo or 5-iodo-uracil; backbone modifications, methylations, unusual base-pairing combinations such as the isobases isocytidine and isoguanidine. Modifications can also include 3′ and 5′ modifications such as capping and change of stereochemistry. The term also includes aptamers.

As used herein, the term “oxidation protection agent” or “antioxidant” refers to a compound which suppresses the oxidation of other compounds such as peptides.

As used herein, the term “pH-adjusting agent” refers to a chemical compound that is used to adjust the pH of a solution.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “physiological conditions” refers to an aqueous buffer at pH 7.4 and 37° C.

The term “polypeptide” as used herein refers to a chain of at least 2 and up to and including 50 amino acid monomer moieties linked by peptide (amide) linkages. Only for CNP, the sequences having more than 50 amino acids will also be referred to as “polypeptide” for simplification.

As used herein, the term “preservative” refers to a chemical substance that has antimicrobial effects and/or prevents chemical degradation.

As used herein, the term “protein” refers to a chain of more than 50 amino acid monomer moieties linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. It is understood that a polymer may also comprise one or more other chemical groups and/or moieties, such as, for example, one or more functional groups. In certain embodiments, a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, in certain embodiments it has a molecular weight of at most 1000 kDa, such as at most 750 kDa, such as at most 500 kDa, such as at most 300 kDa, such as at most 200 kDa, such as at most 100 kDa.

It is understood that also a protein or a polypeptide is a polymer in which the amino acids are the repeating structural units, even though the side chains of each amino acid may be different.

As used herein, the term “polymeric” or “polymeric moiety” means a reagent or a moiety comprising one or more polymers or polymer moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

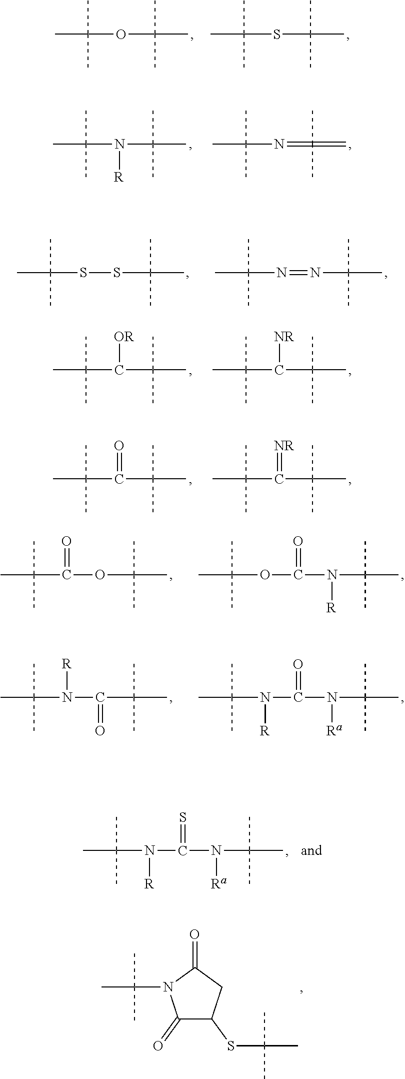

wherein
dashed lines indicate attachment to the remainder of the moiety or reagent, and —R and —R^{a} are independently of each other selected from the group consisting of —H, methyl, ethyl, propyl, butyl, pentyl and hexyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e., to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean number of monomers lies. An integer for “x” given as “about x” means that the arithmetic mean number of monomers lies in a range of integers of x+/−10%, in certain embodiments lies in a range of integers x+/−8%, in certain embodiments lies in a range of integers x+/−5% and in certain embodiments lies in a range of integers x+/−2%.

As used herein, the term “PEG-based” in relation to a moiety or reagent means that said moiety or reagent comprises PEG. In certain embodiments, a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95% (w/w) PEG. The remaining weight percentage of the PEG-based moiety or reagent are other moieties selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

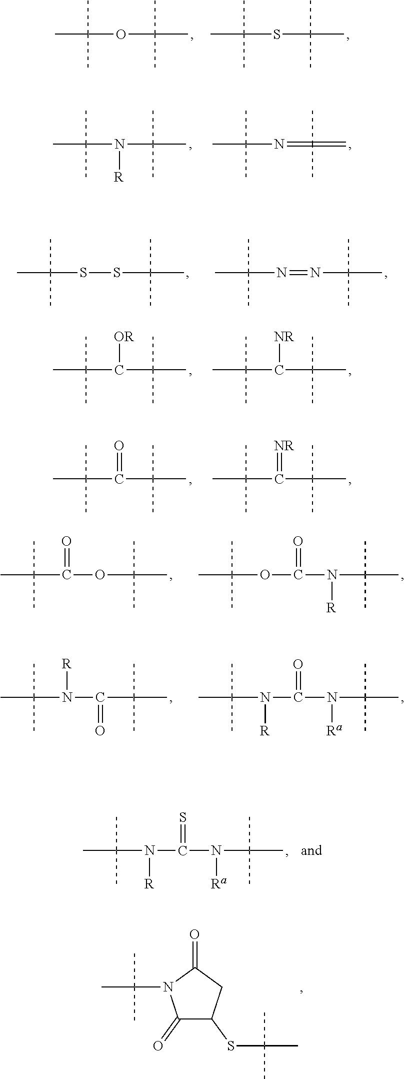

wherein
dashed lines indicate attachment to the remainder of the moiety or reagent, and —R and —R^{a} are independently of each other selected from the group consisting of —H, methyl, ethyl, propyl, butyl, pentyl and hexyl.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent and in certain embodiments, all ethylene glycol units of said moiety or reagent are present in one block; the remaining weight percentage of the PEG-based moiety or reagent are other moieties in certain embodiments selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

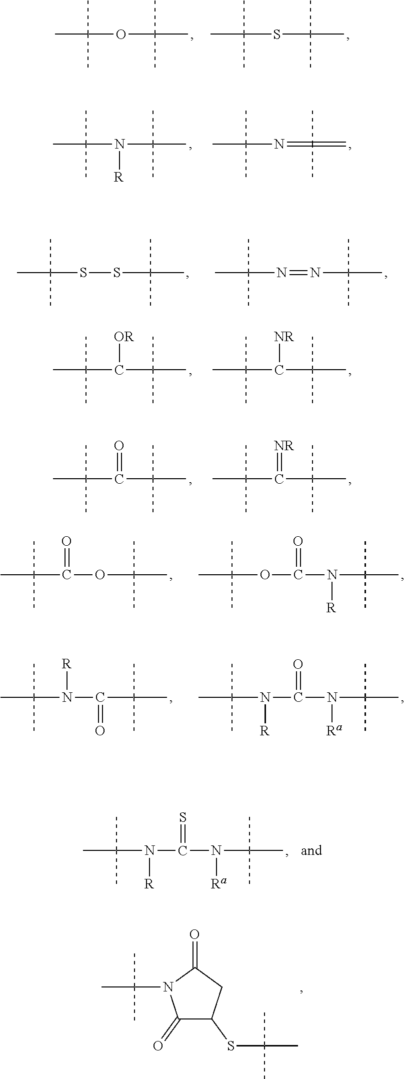

wherein
dashed lines indicate attachment to the remainder of the moiety or reagent, and —R and —R^{a} are independently of each other selected from the group consisting of —H, methyl, ethyl, propyl, butyl, pentyl and hexyl.

As used herein, the term “hyaluronic acid-based comprising at least X % hyaluronic acid” is used accordingly.

It is also recognized by one of ordinary skill in the art that the reversible CNP conjugates comprised within the liquid formulation of the present invention are prodrugs. As used herein, the term “prodrug” refers to a drug moiety, such as a CNP moiety, reversibly and covalently conjugated to a carrier moiety, such as —Z, through a reversible linker moiety. A prodrug releases the reversibly and covalently bound drug moiety in the form of its corresponding drug. In other words, a prodrug is a reversible conjugate comprising a drug moiety, such as a CNP moiety, which is covalently and reversibly conjugated to a carrier moiety via a reversible linker moiety, which covalent and reversible conjugation of the carrier moiety to the reversible linker moiety is either direct or through a spacer. Such prodrugs or conjugates release the formerly conjugated drug moiety in the form of a free drug.

As used herein, the term “random coil” refers to a peptide or protein adopting/having/forming, in certain embodiments having, a conformation which substantially lacks a defined secondary and tertiary structure as determined by circular dichroism spectroscopy performed in aqueous buffer at ambient temperature, and pH 7.4. In certain embodiments, the ambient temperature is about 20° C., i.e. between 18° C. and 22° C., while in certain embodiments the ambient temperature is 20° C.

As used herein, the term “reversible linkage” is a linkage that is cleavable, in the absence of enzymes under physiological conditions (aqueous buffer at pH 7.4, 37° C.) with a half-life ranging from one hour to six months, such as from one hour to four months, such as from one hour to three months, from one hour to two months or from one hour to one month. Accordingly, a stable linkage is a linkage having a half-life under physiological conditions (aqueous buffer at pH 7.4, 37° C.) of more than six months.

As used herein, the term “reagent” means a chemical compound which comprises at least one functional group for reaction with the functional group of another chemical compound or drug. It is understood that a drug comprising a functional group (such as a primary or secondary amine or hydroxyl functional group) is also a reagent.

As used herein, the term “reversible linker moiety” is a moiety which is covalently conjugated to a drug moiety, such as a CNP moiety, through a reversible linkage and is also covalently conjugated to a carrier moiety, such as —Z, wherein the covalent conjugation to said carrier moiety is either direct or through a spacer moiety, such as -L²-. In certain embodiments, the linkage between —Z and -L²- is a stable linkage.

As used herein, the term “reconstitution” means the addition of a liquid to a dry pharmaceutical formulation to bring back the original form of a formulation, such as a solution.

As used herein, the term “small molecule” or “small drug molecule” refers to molecules or drugs that are organic compounds with a molecular weight of less than 1000 Da, such as less than 900 Da or less than 800 Da.

As used herein, the term “sealing a container” means that the container is closed in such way that it is airtight, allowing no gas exchange between the outside and the inside and keeping the content sterile.

As used herein, the term “spacer” or “spacer moiety” refers to a moiety suitable for connecting two moieties. Suitable spacers may be selected from the group consisting of C₁₋₅₀ alkyl, C₂₋₅₀ alkenyl or C₂₋₅₀ alkynyl, which C₁₋₅₀ alkyl, C₂₋₅₀ alkenyl or C₂₋₅₀ alkynyl is optionally interrupted by one or more groups selected from —NH—, —N(C₁₋₄ alkyl)-, —O—, —S—, —C(O)—, —C(O) NH—, —C(O)N(C₁₋₄ alkyl)-, —O—C(O)—, —S(O)—, —S(O)₂—, 4- to 7-membered heterocyclyl, phenyl and naphthyl.

As used herein, the term “substituted” means that one or more-H atom(s) of a molecule or moiety are replaced by a different atom or a group of atoms, which are referred to as “substituent”.

In certain embodiments, such one or more substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O) OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
—R^{x1}, —R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the one or more substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O) OR^{x1a}, —N(R^{x1})C(O)N(R^{x1a}R^{x1b}), —OC(O)N(R^{x1}R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;

each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, a —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the one or more substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O) OR^{x1a}, —N(R^{x1})C(O)N(R^{x1a}R^{x1b}), —OC(O)N(R^{x1}R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3});
each —R^{x1}, —R^{x1a}, —R^{x1b}, —R^{x2}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different.

In certain embodiments, a maximum of 6-H atoms of an optionally substituted molecule are independently replaced by a substituent, e.g., 5-H atoms are independently replaced by a substituent, 4-H atoms are independently replaced by a substituent, 3-H atoms are independently replaced by a substituent, 2-H atoms are independently replaced by a substituent, or 1-H atom is replaced by a substituent.

As used herein, the term “stable” and “stability” with regards to a pharmaceutical formulation comprising a reversible CNP conjugate means that after a storage time, such as after one month, two months, four months, six months, eight months, twelve months, eighteen months, twenty-four months, thirty-six months, forty-eight months, sixty months, in particular after the indicated storage time, the pharmaceutical formulation comprises less than 5% of CNP in its free form and less than 20%, such as less than 10% or such as less than 5% of impurities, such as impurities resulted from the oxidation of methionine. Such storage preferably occurs at 2 to 8° C., i.e., with the formulation in liquid form. Impurities may be quantified by RP-HPLC or SEC based on their respective peak area relative to the total peak area of all CNP-compounds related peaks, particularly of the CNP conjugate-related peaks in the chromatograms and impurities in the CNP moiety and the CNP moiety of the reversible CNP conjugate may be determined by proteolytic digest or peptide mapping and quantified as based on their respective peak area relative to the peak area of the corresponding unmodified proteolytic peptide. Accordingly, the term “stable” and “stability” with regards to a pharmaceutical formulation comprising a CNP agonist or stable CNP conjugate means that after a storage time, such as after one month, two months, four months, six months, eight months, twelve months, eighteen months, twenty-four months, thirty-six months, forty-eight months, sixty months, in particular after the indicated storage time, the pharmaceutical formulation comprises less than 20%, such as less than 10% or such as less than 5% of impurities, such as impurities resulted from the oxidation of methionine. Impurities may be quantified by RP-HPLC or SEC based on their respective peak area relative to the total peak area of all CNP agonists or stable CNP conjugates related peaks, particularly of the CNP conjugate-related peaks in the chromatograms and impurities in the CNP agonist may be determined by proteolytic digest or peptide mapping and quantified as based on their respective peak area relative to the peak area of the corresponding unmodified proteolytic peptide.

As used herein, the term “stabilizer” refers to compounds used to stabilize the CNP compound. Stabilization may be achieved by strengthening of the peptide-stabilizing forces or by direct binding of excipients to the CNP compound.

As used herein, the term “surfactant” refers to wetting agents that lower the surface tension of a liquid.

As used herein, the term “therapeutically effective amount” means an amount sufficient to cure, alleviate or partially arrest the clinical manifestations of a given disease and its complications. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician. Within the scope of this invention, therapeutically effective amount relates to dosages that aim to achieve therapeutic effect for an extended period of time, i.e., for at least one day, such as for two days, such as for three days, such as for four days, such as for five days, such as for six days, such as for one week or such as for two weeks.

As used herein, the term “traceless linker” means a reversible linker which upon cleavage releases the drug in its free form.

As used herein, the term “unit dose” means the amount of the drug administered to a patient in a single dose.

As used herein, the term “water-soluble” with reference to a carrier moiety such as a polymeric moiety means that when such moiety is part of the CNP conjugate, at least 1 g of the CNP conjugate comprising such water-soluble carrier moiety can be dissolved in one liter of water at 20° C. to form a homogeneous solution.

In general, the term “comprise” or “comprising” also encompasses “consist of”, “consisting of”, “consist essentially of” or “consisting essentially of”.

When the disclosure refers to a range of values for a parameter, the disclosure should be understood as also disclosing integers within the range and subranges formed between integers within the range.

In certain embodiments, the CNP compound is a CNP conjugate.

In certain embodiments, the CNP compound is a reversible CNP conjugate, wherein the CNP conjugate comprises at least one CNP moiety that is covalently and reversibly conjugated to a carrier moiety and from which at least one CNP moiety is released with a release half-life of at least 6 hours under physiological conditions.

In certain embodiments, the CNP compound is a stable CNP conjugate, wherein the CNP conjugate comprises at least one CNP moiety that is covalently and stably conjugated to a carrier moiety, i.e. via a stable linkage having a half-life under physiological conditions of more than six months.

In certain embodiments, the CNP compound is a stable CNP conjugate, wherein the CNP conjugate comprises a CNP moiety that is covalently and stably conjugated to a fatty acid derivative.

In certain embodiments, the CNP moiety has a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO: 12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO: 23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO: 34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO: 45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO: 56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO: 67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO: 78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO: 89, SEQ ID NO:90, SEQ ID NO:91, SEQ ID NO:92, SEQ ID NO:93, SEQ ID NO:94 and SEQ ID NO:95.

In certain embodiments, the CNP moiety has the sequence of SEQ ID NO:9, SEQ ID NO:10, SEQ ID NO:11, SEQ ID NO:12, SEQ ID NO:13, SEQ ID NO: 14, SEQ ID NO:15, SEQ ID NO: 16, SEQ ID NO:17, SEQ ID NO: 18, SEQ ID NO: 19, SEQ ID NO:20, SEQ ID NO:21, SEQ ID NO:22, SEQ ID NO:23, SEQ ID NO:24, SEQ ID NO:25 or SEQ ID NO:30. In certain embodiments, the CNP moiety has the sequence of SEQ ID NO:20, SEQ ID NO:21, SEQ ID NO:22, SEQ ID NO:23, SEQ ID NO:24, SEQ ID NO:25 or SEQ ID NO:30. In certain embodiments, the CNP moiety has the sequence of SEQ ID NO:20. In certain embodiments, the CNP moiety has the sequence of SEQ ID NO:21. In certain embodiments, the CNP moiety has the sequence of SEQ ID NO:22. In certain embodiments, the CNP moiety has the sequence of SEQ ID NO:23. In certain embodiments, the CNP moiety has the sequence of SEQ ID NO:24. In certain embodiments, the CNP moiety has the sequence of SEQ ID NO:25. In certain embodiments, the CNP moiety has the sequence of SEQ ID NO:30.

In certain embodiments, the ring moiety of the CNP moiety has the SEQ ID NO:96 or SEQ ID NO: 97. In certain embodiments, the ring moiety of the CNP moiety has the SEQ ID NO: 96. In certain embodiments, the ring moiety of the CNP moiety has the SEQ ID NO:97.

In certain embodiments, the carrier moiety is a polymeric moiety defined as variable-Z, which is described in more detail elsewhere herein.

In order to maintain a certain pH or pH range, the liquid pharmaceutical formulation comprises a buffering agent. The buffering agent maintains the pH of the liquid pharmaceutical formulation within a desired range.

The buffering agent may be selected from the group consisting of acetic acid, succinic acid, citric acid, lactic acid, glutamic acid, fumaric acid, aspartic acid, glutaric acid, phosphoric acid, histidine, gluconic acid, tartaric acid, malic acid, Tris (tris (hydroxymethyl) aminomethane) and mixtures thereof. It is clear to the person skilled in the art that the corresponding conjugate bases or salts of the buffering agents such as acetate, succinate, citrate, lactate, glutamate, fumarate, aspartate, glutarate, phosphate, gluconate, tartrate, malate and mixtures thereof are also included.

In certain embodiments, the buffering agent is selected from the group consisting of acetic acid, succinic acid, citric acid, lactic acid, glutamic acid, fumaric acid, aspartic acid, glutaric acid, phosphoric acid, histidine, gluconic acid, tartaric acid and malic acid.

In certain embodiments, the buffering agent is acetic acid. In certain embodiments, the buffering agent is succinic acid. In certain embodiments, the buffering agent is citric acid. In certain embodiments, the buffering agent is lactic acid. In certain embodiments, the buffering agent is glutamic acid. In certain embodiments, the buffering agent is fumaric acid. In certain embodiments, the buffering agent is aspartic acid. In certain embodiments, the buffering agent is glutaric acid. In certain embodiments, the buffering agent is phosphoric acid. In certain embodiments, the buffering agent is histidine. In certain embodiments, the buffering agent is gluconic acid. In certain embodiments, the buffering agent is tartaric acid. In certain embodiments, the buffering agent is malic acid.

Acetic acid was found to be a particularly useful buffering agent as its use under long term storage does not result in the formation of acetylated adduct impurities as detected by RP-HPLC and/or RP-HPLC-MS.

In certain embodiments, the buffering agent is not citric acid.

In certain embodiments, the buffering agent has a concentration ranging from 5 to 50 mM. In certain embodiments, the buffering agent has a concentration ranging from 10 to 50 mM. In certain embodiments, the buffering agent has a concentration of about 10 mM.

In certain embodiments, the pH of the liquid pharmaceutical formulation is not higher than 6, as under basic conditions the reversible linkage within the reversible CNP conjugate may not be stable.

In certain embodiments, the pH of the liquid pharmaceutical formulation is from about pH 3.5 to about pH 5.5. In certain embodiments, the pH of the liquid pharmaceutical formulation is from about pH 3.5 to about pH 5.0. In certain embodiments, the pH of the liquid pharmaceutical formulation is from about pH 4.5 to about pH 5.0. In certain embodiments, the pH of the liquid pharmaceutical formulation is from about pH 4.0 to about pH 4.5. In certain embodiments, the pH of the liquid pharmaceutical formulation is about 4.5. In certain embodiments, the pH of the liquid pharmaceutical formulation is about 4.0. In certain embodiments, the pH of the liquid pharmaceutical formulation is 4.5. In certain embodiments, the pH of the liquid pharmaceutical formulation is 4.0.

The liquid pharmaceutical formulation according to the present invention comprises an isotonicity agent. The isotonicity agent may be selected from the group consisting of mannitol, trehalose, sucrose, raffinose, gelatin, lactose, dibasic calcium phosphate, sorbitol, xylitol, glycine, histidine, ethanol, hydroxyethylstarch, potassium chloride, sodium chloride, dextrose, dextran, Ficoll®, propylene glycol, glycerol and mixtures thereof.

In certain embodiments, the isotonicity agent is selected from the group consisting of mannitol, trehalose, sucrose, raffinose, gelatin, lactose, dibasic calcium phosphate, sorbitol, xylitol, glycine, histidine, ethanol, hydroxyethylstarch, potassium chloride, sodium chloride, dextrose, dextran, propylene glycol, glycerol and mixtures thereof.

In certain embodiments, the isotonicity agent is selected from the group consisting of mannitol, trehalose, sucrose, raffinose, gelatin, lactose, dibasic calcium phosphate, sorbitol, xylitol, glycine, histidine, ethanol, hydroxyethylstarch, potassium chloride, sodium chloride, dextrose, dextran, glycerol and propylene glycol.

In certain embodiments, the isotonicity agent is mannitol. In certain embodiments, the isotonicity agent is trehalose, such as trehalose dihydrate. In certain embodiments, the isotonicity agent is sucrose. In certain embodiments, the isotonicity agent is raffinose. In certain embodiments, the isotonicity agent is gelatin. In certain embodiments, the isotonicity agent is lactose. In certain embodiments, the isotonicity agent is dibasic calcium phosphate. In certain embodiments, the isotonicity agent is sorbitol. In certain embodiments, the isotonicity agent is xylitol. In certain embodiments, the isotonicity agent is glycine. In certain embodiments, the isotonicity agent is histidine. In certain embodiments, the isotonicity agent is ethanol. In certain embodiments, the isotonicity agent is hydroxyethylstarch. In certain embodiments, the isotonicity agent is potassium chloride. In certain embodiments, the isotonicity agent is sodium chloride. In certain embodiments, the isotonicity agent is dextrose. In certain embodiments, the isotonicity agent is dextran. In certain embodiments, the isotonicity agent is Ficoll®. In certain embodiments, the isotonicity agent is propylene glycol.

In certain embodiments, the isotonicity agent is glycerol.

In certain embodiments, the isotonicity agent is selected from the group consisting of mannitol and trehalose.

In certain embodiments, the isotonicity agent is a mixture of mannitol and trehalose.

As defined herein, the term “trehalose” is intended to encompass all salts and hydration states of trehalose, such as trehalose anhydrous or trehalose dihydrate. In certain embodiments, the term “trehalose” refers to trehalose anhydrous. In certain embodiments, the term “trehalose” refers to trehalose dihydrate.

As defined herein, the term “mannitol” is intended to encompass both D-mannitol and L-mannitol, and mixtures thereof. In certain embodiments, the term “mannitol” refers to L-mannitol. In certain embodiments, the term “mannitol” refers to D-mannitol. In certain embodiments, the term “mannitol” refers to a mixture of L-mannitol and D-mannitol.

In certain embodiments, the isotonicity agent has a concentration ranging from 10 to 200 mg/ml. In certain embodiments, the isotonicity agent has a concentration of about 100 mg/ml.

In certain embodiments, the isotonicity agent has a concentration of about 84 mg/ml. In certain embodiments, the isotonicity agent has a concentration of about 80 mg/ml. In certain embodiments, the isotonicity agent has a concentration of about 50 mg/ml. In certain embodiments, the isotonicity agent has a concentration of about 41 mg/ml.

In some embodiments, the liquid formulation is substantially isotonic implying an osmolality of about 250-350 mOsm/kg water. Liquid formulations can also be hypertonic (>350 mOsm/kg water) or hypotonic (<250 mOsm/kg water).

In certain embodiments, reducing sugars should be avoided, as they may react with the CNP moiety. Accordingly, in certain embodiments the pharmaceutical formulation does not comprise a reducing sugar.

The liquid pharmaceutical formulation according to the present invention comprises a preservative. The preservative may be selected from the group consisting of m-cresol, benzyl alcohol, benzoic acid, phenol, methylparaben, ethylparaben, propylparaben, butylparaben, potassium sorbate, chlorobutanol, benzyl alcohol, phenylmercuric nitrate, thimerosal, sorbic acid, potassium sorbate, chlorocresol, benzalkonium chloride, 2-ethoxyethanol, chlorhexidine, chlorobutanol, phenylethyl alcohol, phenylmercuric acetate and mixtures thereof.

In certain embodiments, the preservative is m-cresol. In certain embodiments, the preservative is benzyl alcohol. In certain embodiments, the preservative is benzoic acid. In certain embodiments, the preservative is phenol. In certain embodiments, the preservative is methylparaben. In certain embodiments, the preservative is ethylparaben. In certain embodiments, the preservative is propylparaben. In certain embodiments, the preservative is butylparaben. In certain embodiments, the preservative is potassium sorbate. In certain embodiments, the preservative is chlorocresol. In certain embodiments, the preservative is benzyl alcohol. In certain embodiments, the preservative is phenylmercuric nitrate. In certain embodiments, the preservative is thimerosal. In certain embodiments, the preservative is sorbic acid. In certain embodiments, the preservative is potassium sorbate. In certain embodiments, the preservative is chlorocresol. In certain embodiments, the preservative is benzalkonium chloride. In certain embodiments, the preservative is 2-ethoxyethanol. In certain embodiments, the preservative is chlorhexidine. In certain embodiments, the preservative is chlorobutanol. In certain embodiments, the preservative is phenylethyl alcohol. In certain embodiments, the preservative is phenylmercuric acetate.

In certain embodiments, the preservative has a concentration ranging from 1 to 6 mg/ml. In certain embodiments, the preservative has a concentration ranging from 3 to 6 mg/ml. In certain embodiments, the preservative has a concentration ranging from 2 to 3 mg/ml. In certain embodiments, the preservative has a concentration of about 3 mg/ml. In certain embodiments, the preservative has a concentration of 3 mg/ml.

The liquid pharmaceutical formulation according to the present invention may further comprise a pH-adjusting agent. In certain embodiments, the pH-adjusting agent is a base. Examples of bases may be selected from the group consisting of sodium hydroxide, Tris (tris (hydroxymethyl) aminomethane), potassium hydroxide, lysine, and mixtures thereof.

In certain embodiments, the pH-adjusting agent is a base selected from the group consisting of sodium hydroxide, sodium acetate, Tris (tris (hydroxymethyl) aminomethane), potassium hydroxide and lysine.

In certain embodiments, the pH-adjusting agent is selected from the group consisting of sodium hydroxide and Tris (tris (hydroxymethyl) aminomethane).

In certain embodiments, the pH-adjusting agent is sodium hydroxide. In certain embodiments, the pH-adjusting agent is sodium acetate. In certain embodiments, the pH-adjusting agent is Tris. In certain embodiments, the pH-adjusting agent is potassium hydroxide. In certain embodiments, the pH-adjusting agent is lysine.

In certain embodiments, the pH-adjusting agent is an acid. Examples of acids may be selected from the group consisting of hydrochloric acid, phosphoric acid, carbonic acid, nitric acid and mixtures thereof.

In certain embodiments, the pH-adjusting agent is an acid selected from the group consisting of hydrochloric acid, phosphoric acid, carbonic acid and nitric acid.

In certain embodiments, the pH-adjusting agent is hydrochloric acid. In certain embodiments, the pH-adjusting agent is phosphoric acid. In certain embodiments, the pH-adjusting agent is carbonic acid. In certain embodiments, the pH-adjusting agent is nitric acid.

In certain embodiment, the pH-adjusting agent is a mixture of at least one base and at least one acid. In certain embodiment, the pH-adjusting agent is a mixture of one base and one acid. In certain embodiment, the pH-adjusting agent is a mixture of sodium hydroxide and hydrochloric acid.

In certain embodiments, the pH-adjusting agent or mixture of pH-adjusting agents has a concentration ranging from 0.01 to 5 mg/ml. In certain embodiments, the pH-adjusting agent or mixture of pH-adjusting agents has a concentration ranging from 0.04 to 2.5 mg/ml. In certain embodiments, the pH-adjusting agent or mixture of pH-adjusting agents has a concentration ranging from 0.08 to 1.25 mg/ml. In certain embodiments, the pH-adjusting agent or mixture of pH-adjusting agents has a concentration of about 0.4 mg/ml. It is understood that in case of a mixture of pH-adjusting agents the provided concentrations refer to the total concentration of all pH-adjusting agents.

The liquid pharmaceutical formulation according to the present invention optionally comprises an antioxidant. Examples of antioxidants may be selected from the group consisting of methionine, butylhydroxytoluene, butylhydroxyanisole, tocopherol, propylgallate, ascorbic acid, sodium bisulfite, ethylenediaminetetraacetic acid (EDTA), cysteine, glutathione, monothioglycerol, poly(ethylenimine), vitamin E, ectoine, morin and mixtures thereof.

In certain embodiments, the antioxidant is selected from the group consisting of methionine, butylhydroxytoluene, butylhydroxyanisole, tocopherol, propylgallate, ascorbic acid, sodium bisulfite, ethylenediaminetetraacetic acid (EDTA), cysteine, glutathione, monothioglycerol, poly(ethylenimine), vitamin E, ectoine and morin.

It was observed that methionine is particularly useful in preventing the oxidation of the residues, such as of the methionine residues within the CNP compound to, for example methionine sulfoxide or methionine sulfone.

In certain embodiments, the antioxidant is methionine. In certain embodiments, the antioxidant is ascorbic acid. In certain embodiments, the antioxidant is butylhydroxytoluene. In certain embodiments, the antioxidant is butylhydroxyanisole. In certain embodiments, the antioxidant is tocopherol. In certain embodiments, the antioxidant is propylgallate. In certain embodiments, the antioxidant is sodium bisulfite. In certain embodiments, the antioxidant is monothioglycerol. In certain embodiments, the antioxidant is EDTA. In certain embodiments, the antioxidant is cysteine. In certain embodiments, the antioxidant is glutathione. In certain embodiments, the antioxidant is poly(ethylenimine). In certain embodiments, the antioxidant is vitamin E. In certain embodiments, the antioxidant is ectoine. In certain embodiments, the antioxidant is morin.

As defined herein, the term “methionine” is intended to encompass both D-methionine and L-methionine, and mixtures thereof. In certain embodiments, the term “methionine” refers to L-methionine. In certain embodiments, the term “methionine” refers to D-methionine. In certain embodiments, the term “methionine” refers to a mixture of D-methionine or L-methionine.

As defined herein, the term “EDTA” is intended to encompass all EDTA forms that are known in the art such as EDTA salts, including EDTA metal salts, such as EDTA disodium salt, EDTA dipotassium salt, EDTA calcium salt, EDTA dimagnesium salt or mixtures thereof. In certain embodiments, EDTA refers to EDTA disodium salt. In certain embodiments, the term “EDTA” refers to EDTA dicalcium salt. In certain embodiments, the term “EDTA” refers to EDTA anhydrous.

In certain embodiments, the liquid pharmaceutical formulation does not comprise sodium bisulfite. In certain embodiments, the liquid pharmaceutical formulation does not comprise cysteine. In certain embodiments, the liquid pharmaceutical formulation does not comprise glutathione. In certain embodiments, the liquid pharmaceutical formulation does not comprise monothioglycerol. In certain embodiments, the liquid pharmaceutical formulation does not comprise ascorbic acid.

In certain embodiments, the antioxidant has a concentration ranging from about 0.1 mg/ml to about 1.5 mg/ml. In certain embodiments, the antioxidant has a concentration ranging from about 0.5 mg/ml to about 1.0 mg/ml.

In certain embodiments, the molar ratio of antioxidant to CNP or CNP agonist is from about 0.1:1 to about 100:1. In certain embodiments, the molar ratio of antioxidant to CNP or CNP agonist is from about 0.1:1 to about 70:1. In certain embodiments, the molar ratio of antioxidant to CNP or CNP agonist is from about 0.1:1 to about 15:1. In certain embodiments, the molar ratio of antioxidant to CNP or CNP agonist is from about 1:1 to about 10:1. In certain embodiments, the molar ratio of antioxidant to CNP or CNP agonist is from about 3:1 to about 7:1.

In certain embodiments, the liquid pharmaceutical formulation of the present invention comprises no antioxidant.

The liquid pharmaceutical formulation according to the present invention optionally comprises a detergent. Examples of detergents may be selected from the group consisting of octylsucrose, Triton X-100, Triton X-114, polysorbate-20, polysorbate-80, NP-40, CA-630, Brij-35, Brij-58, n-dodecyl-beta-maltoside, octyl-beta-glucoside, octylthioglucoside, sodium dodecyl sulfate, CHAPS, Pluronic® F-127 and Pluronic® F-68.

In certain embodiments, the liquid pharmaceutical formulation of the present invention comprises no detergent.

Formulations intended for administration to humans are preferably made under good manufacturing practices (GMP) approved or approvable by the FDA or a regulatory agency for a country other than the United States, for example, the European Medicines Agency, for preparation of drugs for administration to humans. Typically, the formulations are sterile, for example, as accomplished by sterile filtration using a 0.2 μm or a 0.22 μm filter.

In certain embodiments, the liquid pharmaceutical formulation of the present invention comprises a CNP agonist in a concentration of from about 0.1 to about 20 mg/ml. In certain embodiments, the concentration of the CNP agonist is from about 0.5 to about 15 mg/ml. In certain embodiments, the concentration of the CNP agonist is from about 0.75 to about 10 mg/ml. In certain embodiments, the concentration of the CNP agonist is from about 1 to about 8 mg/ml. In certain embodiments, the concentration of the CNP agonist is from about 2 to about 6 mg/ml. In certain embodiments, the concentration of the CNP agonist is about 10 mg/ml. In certain embodiments, the concentration of the CNP agonist is about 8 mg/ml. In certain embodiments, the concentration of the CNP agonist is about 6 mg/ml. In certain embodiments, the concentration of the CNP agonist is about 4 mg/ml. In certain embodiments, the concentration of the CNP agonist is about 2 mg/ml. In certain embodiments, the concentration of the CNP agonist is about 1 mg/ml.

In certain embodiments, the liquid pharmaceutical formulation of the present invention comprises a CNP agonist in a concentration of from 0.1 to 20 mg/ml. In certain embodiments, the concentration of the CNP agonist of from 0.5 to 15 mg/ml. In certain embodiments, the concentration of the CNP agonist is of from 0.75 to 10 mg/ml. In certain embodiments, the concentration of the CNP agonist is of from 1 to 8 mg/ml. In certain embodiments, the concentration of the CNP agonist is of from 2 to 6 mg/ml. In certain embodiments, the concentration of the CNP agonist is 10 mg/ml. In certain embodiments, the concentration of the CNP agonist is 8 mg/ml. In certain embodiments, the concentration of the CNP agonist is 6 mg/ml. In certain embodiments, the concentration of the CNP agonist is 4 mg/ml. In certain embodiments, the concentration of the CNP agonist is 2 mg/ml. In certain embodiments, the concentration of the CNP agonist is 1 mg/ml.

In certain embodiments, the liquid pharmaceutical formulation of the present invention comprises a CNP conjugate of which the CNP moiety is present in a concentration of from about 0.1 to about 20 mg/ml. In certain embodiments, the liquid pharmaceutical formulation comprises a CNP conjugate of which the CNP moiety is present in a concentration of from about 0.5 to about 15 mg/ml. In certain embodiments, the liquid pharmaceutical formulation comprises a CNP conjugate of which the CNP moiety is present in a concentration of from about 0.75 to about 10 mg/ml. In certain embodiments, the liquid pharmaceutical formulation comprises a CNP conjugate of which the CNP moiety is present in a concentration of from about 1 to about 8 mg/ml. In certain embodiments, the liquid pharmaceutical formulation comprises a CNP conjugate of which the CNP moiety is present in a concentration of from about 2 to about 6 mg/ml. In certain embodiments, the liquid pharmaceutical formulation comprises a CNP conjugate of which the CNP moiety is present in a concentration of about 10 mg/ml. In certain embodiments, the liquid pharmaceutical formulation comprises a CNP conjugate of which the CNP moiety is present in a concentration of about 8 mg/ml. In certain embodiments, the liquid pharmaceutical formulation comprises a CNP conjugate of which the CNP moiety is present in a concentration of about 6 mg/ml. In certain embodiments, the liquid pharmaceutical formulation comprises a CNP conjugate of which the CNP moiety is present in a concentration of about 4 mg/ml. In certain embodiments, the liquid pharmaceutical formulation comprises a CNP conjugate of which the CNP moiety is present in a concentration of about 2 mg/ml. In certain embodiments, the liquid pharmaceutical formulation comprises a CNP conjugate of which the CNP moiety is present in a concentration of about 1 mg/ml. It is understood that the above provided concentrations refer to the amount of the CNP moiety, but not to the whole CNP conjugate.

In certain embodiments, the liquid pharmaceutical formulation of the present invention comprises a CNP conjugate of which the CNP moiety is present in a concentration of from 0.1 to 20 mg/ml. In certain embodiments, the liquid pharmaceutical formulation comprises a CNP conjugate of which the CNP moiety is present in a concentration of from 0.5 to 15 mg/ml. In certain embodiments, the liquid pharmaceutical formulation comprises a CNP conjugate of which the CNP moiety is present in a concentration of from 0.75 to 10 mg/ml. In certain embodiments, the liquid pharmaceutical formulation comprises a CNP conjugate of which the CNP moiety is present in a concentration of from 1 to 8 mg/ml. In certain embodiments, the liquid pharmaceutical formulation comprises a CNP conjugate of which the CNP moiety is present in a concentration of from 2 to 6 mg/ml. In certain embodiments, the liquid pharmaceutical formulation comprises a CNP conjugate of which the CNP moiety is present in a concentration of 10 mg/ml. In certain embodiments, the liquid pharmaceutical formulation comprises a CNP conjugate of which the CNP moiety is present in a concentration of 8 mg/ml. In certain embodiments, the liquid pharmaceutical formulation comprises a CNP conjugate of which the CNP moiety is present in a concentration of 6 mg/ml. In certain embodiments, the liquid pharmaceutical formulation comprises a CNP conjugate of which the CNP moiety is present in a concentration of 4 mg/ml. In certain embodiments, the liquid pharmaceutical formulation comprises a CNP conjugate of which the CNP moiety is present in a concentration of 2 mg/ml. In certain embodiments, the liquid pharmaceutical formulation comprises a CNP conjugate of which the CNP moiety is present in a concentration of 1 mg/ml. It is understood that the above provided concentrations refer to the amount of the CNP moiety, but not to the whole CNP conjugate.

In certain embodiments, the reversible CNP conjugate is of formula (Ia) or (Ib):

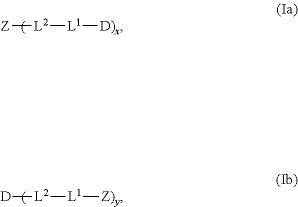

wherein
-D is a CNP moiety;
-L¹- is a reversible linker moiety;
-L²- is a single chemical bond or a spacer moiety;
—Z is a carrier moiety;
x is an integer selected from the group consisting of 1, 2, 3, 4, 5, 6, 7, 8, 9, 10, 11, 12, 13, 14, 15 and 16; and
y is an integer selected from the group consisting of 2, 3, 4 and 5.
-D of formula (Ia) or (Ib) is covalently and reversibly conjugated to -L¹-.

In certain embodiments, the liquid pharmaceutical formulation of the present invention comprises:

| | | |
|---|---|---|
| CNP conjugate, of which CNP moiety | 0.1-20 | mg/ml |
| acetic acid | 0.3-3 | mg/ml |
| D-mannitol | 10-200 | mg/ml |
| m-cresol | 1-10 | mg/ml. |

In certain embodiments, the liquid pharmaceutical formulation of the present invention comprises:

| | | |
|---|---|---|
| CNP conjugate, of which CNP moiety | 0.1-20 | mg/ml |
| acetic acid | 0.3-3 | mg/ml |
| trehalose dihydrate | 10-200 | mg/ml |
| m-cresol | 1-10 | mg/ml. |

In certain embodiments, —Z of formula (Ia) or (Ib) is a polymeric moiety. In certain embodiments, —Z of formula (Ia) or (Ib) is a water-soluble polymeric moiety.

In certain embodiments, x of formula (Ia) is an integer selected from the group consisting of 1, 2, 3, 4, 6 and 8. In certain embodiments, x of formula (Ia) is an integer selected from the group consisting of 1, 2, 4 and 6. In certain embodiments, x of formula (Ia) is an integer selected from the group consisting of 1, 4 and 6 and in certain embodiments, x of formula (Ia) is 1.

In certain embodiments, y of formula (Ib) is an integer selected from the group consisting of 3, 4 and 5. In certain embodiments, y of formula (Ib) is an integer selected from the group consisting of 4 and 5. In certain embodiments, y of formula (Ib) is an integer selected from the group consisting of 2 and 3. In certain embodiments, y of formula (Ib) is 2. In certain embodiments, y of formula (Ib) is 3. In certain embodiments, y of formula (Ib) is 4. In certain embodiments, y of formula (Ib) is 5.

In certain embodiments, the reversible CNP conjugate is of formula (Ia) with x=1.

In certain embodiments, -D of formula (Ia) or (Ib) has the sequence of SEQ ID NO:9, SEQ ID NO: 10, SEQ ID NO:11, SEQ ID NO: 12, SEQ ID NO:13, SEQ ID NO:14, SEQ ID NO:15, SEQ ID NO:16, SEQ ID NO:17, SEQ ID NO:18, SEQ ID NO: 19, SEQ ID NO:20, SEQ ID NO: 21, SEQ ID NO:22, SEQ ID NO:23, SEQ ID NO:24, SEQ ID NO:25 or SEQ ID NO:30. In certain embodiments, -D of formula (Ia) or (Ib) has the sequence of SEQ ID NO:20, SEQ ID NO: 21, SEQ ID NO:22, SEQ ID NO:23, SEQ ID NO:24 or SEQ ID NO:25.

In certain embodiments, -D of formula (Ia) or (Ib) has the sequence of SEQ ID NO:20. In certain embodiments, -D of formula (Ia) or (Ib) has the sequence of SEQ ID NO:21. In certain embodiments, -D of formula (Ia) or (Ib) has the sequence of SEQ ID NO:22. In certain embodiments, -D of formula (Ia) or (Ib) has the sequence of SEQ ID NO:23. In certain embodiments, -D of formula (Ia) or (Ib) has the sequence of SEQ ID NO:24. In certain embodiments, -D of formula (Ia) or (Ib) has the sequence of SEQ ID NO:25.

The moiety -L¹- of formula (Ia) or (Ib) is either conjugated to a functional group of the side chain of an amino acid residue of -D, to the N-terminal amine functional group or to the C-terminal carboxyl functional group of -D or to a nitrogen atom in the backbone polypeptide chain of -D. Attachment to either the N-terminus or C-terminus can either be direct through the corresponding amine or carboxyl functional group, respectively, or indirect wherein a spacer moiety is first conjugated to the amine or carboxyl functional group to which spacer moiety -L¹- is conjugated.

The moiety -L¹- of formula (Ia) or (Ib) is a reversible linker from which the drug, i.e. D-H is released in its free or unmodified form, i.e. -L¹- is a traceless linker. Suitable reversible linkers are known in the art, such as for example the reversible linker moieties disclosed in WO 2005/099768 A2, WO 2006/136586 A2, WO 2011/089216 A1 and WO 2013/024053 A1, which are incorporated by reference herewith.

In certain embodiments, -L¹- is a reversible linker as described in WO 2011/012722 A1, WO 2011/089214 A1, WO 2011/089215 A1, WO 2013/024052 A1 and WO 2013/160340 A1 which are incorporated by reference herewith.

The moiety -L¹- can be connected to -D through any type of linkage, provided that it is reversible. In certain embodiments, -L¹- is connected to -D through a linkage selected from the group consisting of amide, ester, carbamate, acetal, aminal, imine, oxime, hydrazone, disulfide and acylguanidine. In certain embodiments, -L¹- is connected to -D through a linkage selected from the group consisting of amide, ester, carbamate and acylguanidine. It is understood that these linkages may not per se be reversible, but that neighboring groups comprised in -L¹- may render the linkage reversible.

In certain embodiments, the moiety -L¹- is connected to -D through an amide linkage.

A moiety -L¹- is disclosed in WO 2009/095479 A2. Accordingly, in certain embodiments, the moiety -L¹- is of formula (II):

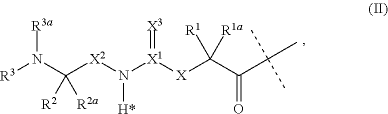

wherein the dashed line indicates the attachment to a nitrogen of -D which is a CNP moiety by forming an amide bond;
—X— is —C(R⁴R^{4a})—, —N(R⁴)—, —O—, —C(R⁴R^{4a})—C(R⁵R^{5a})—, —C(R⁵R^{5a})—C(R⁴R^{4a})—, —C(R⁴R^{4a})—N(R⁶)—, —N(R⁶)—C(R⁴R^{4a})—, —C(R⁴R^{4a})—O—, —O—C(R⁴R^{4a})— or —C(R⁷R^{7a})—;
X¹ is C or S (O);
—X²— is —C(R⁸R^{8a})— or —C(R⁸R^{8a})—C(R⁹R^{9a})—;
═X³ is ═O, ═S or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to the N atom to which they are attached through an sp³-hybridized carbon atom;
—R⁷ is-N(R¹⁰R^{10a}) or —NR¹⁰—(C═O)—R¹¹;
—R^{7a}, —R¹⁰, —R^{10a}, —R¹¹ are independently of each other-H or C₁₋₆ alkyl;
optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁸/—R^{8a}, —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹, —R²/—R³ are joined together with the atoms to which they are attached to form a ring A;
optionally, —R³/—R^{3a} are joined together with the nitrogen atom to which they are attached to form a 3- to 10-membered heterocycle;
A is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; and
wherein -L¹- is substituted with -L²-Z and wherein -L¹- is optionally further substituted, provided that the hydrogen marked with the asterisk in formula (II) is not replaced by -L²-Z or a substituent;
wherein
-L²- is a single chemical bond or a spacer; and
—Z is a carrier moiety, preferably a water-soluble polymeric moiety.

In certain embodiments, -L¹- of formula (II) is substituted with one moiety -L²-Z.

In certain embodiments, -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles, in which the atoms directly attached to the nitrogen are sp³-hybridized carbon atoms may be formed. In other words, such 3- to 10-membered heterocycle formed by —R³/—R^{3a} together with the nitrogen atom to which they are attached has the following structure:

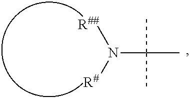

wherein
the dashed line indicates attachment to the rest of -L¹-;
the ring comprises 3 to 10 atoms comprising at least one nitrogen; and
R^{#} and R^{##} represent a sp³-hybridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

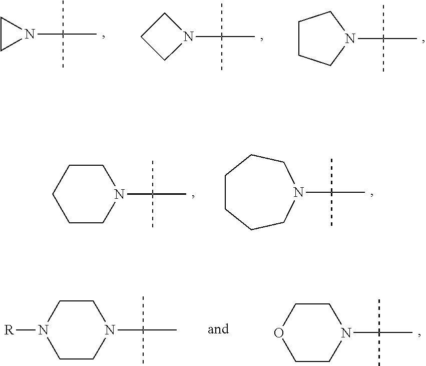

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e., the hydrogen marked with the asterisk in formula (II) is not replaced and the nitrogen of the moiety

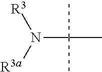

of formula (II) remains part of a primary, secondary or tertiary amine, i.e. —R³ and —R^{3a} are independently of each other-H or are connected to —N< through a sp³-hybridized carbon atom.

In certain embodiments, —R¹ or —R^{1a} of formula (II) is substituted with -L²-Z. In certain embodiments, —R² or —R^{2a} of formula (II) is substituted with -L²-Z. In certain embodiments, —R³ or —R^{3a} of formula (II) is substituted with -L²-Z. In certain embodiments, —R⁴ of formula (II) is substituted with -L²-Z. In certain embodiments, —R⁵ or —R^{5a} of formula (II) is substituted with -L²-Z. In certain embodiments, —R⁶ of formula (II) is substituted with -L²-Z. In certain embodiments, —R⁷ or —R^{7a} of formula (II) is substituted with -L²-Z. In certain embodiments, —R⁸ or —R^{8a} of formula (II) is substituted with -L²-Z. In certain embodiments, —R⁹ or —R^{9a} of formula (II) is substituted with -L²-Z.

In certain embodiments, —R⁴ of formula (II) is substituted with -L²-Z.

In certain embodiments, —X— of formula (II) is —C(R⁴R^{4a})— or —N(R⁴)—. In certain embodiments, —X— of formula (II) is —C(R⁴R^{4a})—.

In certain embodiments, X¹ of formula (II) is C.

In certain embodiments, ═X³ of formula (II) is ═O.

In certain embodiments, —X²— of formula (II) is —C(R⁸R^{8a})—.

In certain embodiments, —R⁸ and —R^{8a} of formula (II) are independently selected from the group consisting of —H, methyl and ethyl. In certain embodiments, at least one of —R⁸ and —R^{8a} of formula (II) is —H. In certain embodiments, both —R⁸ and —R^{8a} of formula (II) are —H.

In certain embodiments, —R¹ and —R^{1a} of formula (II) are independently selected from the group consisting of —H, methyl and ethyl. In certain embodiments, at least one of —R¹ and —R^{1a} of formula (II) is —H. In certain embodiments, both —R¹ and —R^{1a} of formula (II) are —H.

In certain embodiments, —R² and —R^{2a} of formula (II) are independently selected from the group consisting of —H, methyl and ethyl. In certain embodiments, at least one of —R² and —R^{2a} of formula (II) is —H. In certain embodiments, both —R² and —R^{2a} of formula (II) are H.

In certain embodiments, —R³ and —R^{3a} of formula (II) are independently selected from the group consisting of —H, methyl, ethyl, propyl and butyl. In certain embodiments, at least one of —R³ and —R^{3a} of formula (II) is methyl. In certain embodiments, —R³ and —R^{3a} of formula (II) are both —H. In certain embodiments, —R³ and —R^{3a} of formula (II) are both methyl. In certain embodiments, —R³ of formula (II) is —H and —R^{3a} of formula (II) is methyl.

In certain embodiments, —R⁴ and —R^{4a} of formula (II) are independently selected from the group consisting of —H, methyl and ethyl. In certain embodiments, at least one of —R⁴ and —R^{4a} of formula (II) is —H. In certain embodiments, both —R⁴ and —R^{4a} of formula (II) are —H.

In certain embodiments, the moiety -L¹- is of formula (IIa):

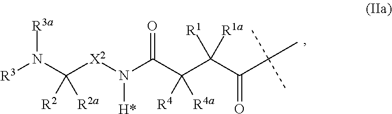

wherein the dashed line indicates the attachment to a nitrogen of -D which is a CNP moiety by forming an amide bond;
—R¹, —R^{1a}, —R², —R^{2a}, —R³, —R^{3a}, —R⁴, —R^{4a} and —X²— are used as defined in formula (II); and wherein -L¹- is substituted with -L²-Z and wherein -L¹- is optionally further substituted, provided that the hydrogen marked with the asterisk in formula (IIa) is not replaced by -L²-Z or a substituent.

In certain embodiments, -L¹- of formula (IIa) is substituted with one moiety -L²-Z.

In certain embodiments, the moiety -L¹- of formula (IIa) is not further substituted.

In certain embodiments, —R¹ and —R^{1a} of formula (IIa) are independently selected from the group consisting of —H, methyl and ethyl. In certain embodiments, at least one of —R¹ and —R^{1a} of formula (IIa) is —H. In certain embodiments, both —R¹ and —R^{1a} of formula (IIa) are —H. In certain embodiments, —R⁴ and —R^{4a} of formula (IIa) are independently selected from the group consisting of —H, methyl and ethyl. In certain embodiments, at least one of —R⁴ and —R^{4a} of formula (IIa) is —H. In certain embodiments, both —R⁴ and —R^{4a} of formula (IIa) are —H.

In certain embodiments, —X²— of formula (IIa) is —C(R⁸R^{8a})—.

In certain embodiments, —R⁸ and —R^{8a} of formula (IIa) are independently selected from the group consisting of —H, methyl and ethyl. In certain embodiments, at least one of —R⁸ and —R^{8a} of formula (IIa) is —H. In certain embodiments, both —R⁸ and —R^{8a} of formula (IIa) are —H.

In certain embodiments, —R² and —R^{2a} of formula (IIa) are independently selected from the group consisting of —H, methyl and ethyl. In certain embodiments, at least one of —R² and —R^{2a} of formula (IIa) is —H. In certain embodiments, both —R² and —R^{2a} of formula (IIa) are H.

In certain embodiments, —R³ and —R^{3a} of formula (IIa) are independently selected from the group consisting of —H, methyl, ethyl, propyl and butyl. In certain embodiments, at least one of —R³ and —R^{3a} of formula (IIa) is methyl. In certain embodiments, —R³ and —R^{3a} of formula (IIa) are both —H. In certain embodiments, —R³ and —R^{3a} of formula (IIa) are both methyl. In certain embodiments, —R³ of formula (IIa) is —H and —R^{3a} of formula (IIa) is methyl.

In certain embodiments, the moiety -L¹- is of formula (IIb):

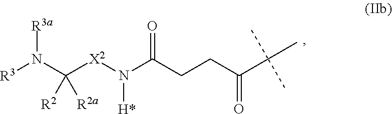

wherein the dashed line indicates the attachment to a nitrogen of -D which is a CNP moiety by forming an amide bond;
—R², —R^{2a}, —R³, —R^{3a} and —X²— are used as defined in formula (II); and
wherein -L¹- is substituted with -L²-Z and wherein -L¹- is optionally further substituted, provided that the hydrogen marked with the asterisk in formula (IIb) is not replaced by -L²-Z or a substituent.

In certain embodiments, -L¹- of formula (IIb) is substituted with one moiety -L²-Z. In certain embodiments, the moiety -L¹- of formula (IIb) is not further substituted.

In certain embodiments, —X²— of formula (IIb) is —C(R⁸R^{8a})—.

In certain embodiments, —R⁸ and —R^{8a} of formula (IIb) are independently selected from the group consisting of —H, methyl and ethyl. In certain embodiments, at least one of —R⁸ and —R^{8a} of formula (IIb) is —H. In certain embodiments, both —R⁸ and —R^{8a} of formula (IIb) are —H.

In certain embodiments, —R² and —R^{2a} of formula (IIb) are independently selected from the group consisting of —H, methyl and ethyl. In certain embodiments, at least one of —R² and —R^{2a} of formula (IIb) is —H. In certain embodiments, both —R² and —R^{2a} of formula (IIb) are H.

In certain embodiments, —R³ and —R^{3a} of formula (IIb) are independently selected from the group consisting of —H, methyl, ethyl, propyl and butyl. In certain embodiments, at least one of —R³ and —R^{3a} of formula (IIb) is methyl. In certain embodiments, —R³ and —R^{3a} of formula (IIb) are both —H. In certain embodiments, —R³ and —R^{3a} of formula (IIb) are both methyl. In certain embodiments, —R³ of formula (IIb) is —H and —R^{3a} of formula (IIb) is methyl.

In certain embodiments, the moiety -L¹- is of formula (IIb′):

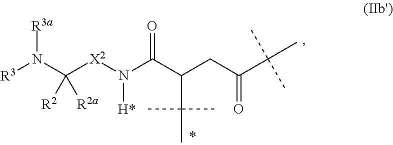

wherein
wherein the dashed line indicates the attachment to a nitrogen of -D which is a CNP moiety by forming an amide bond;
the dashed line marked with the asterisk indicates attachment to -L²-; —R², —R^{2a}, —R³, —R^{3a} and —X²— are used as defined in formula (II); and
wherein -L¹- is optionally further substituted, provided that the hydrogen marked with the asterisk in formula (IIb′) is not replaced by a substituent.

In certain embodiments, the moiety -L¹- of formula (IIb′) is not further substituted.

In certain embodiments, —X²— of formula (IIb′) is —C(R⁸R^{8a})—.

In certain embodiments, —R⁸ and —R^{8a} of formula (IIb′) are independently selected from the group consisting of —H, methyl and ethyl. In certain embodiments, at least one of —R⁸ and —R^{8a} of formula (IIb′) is —H. In certain embodiments, both —R⁸ and —R^{8a} of formula (IIb′) are —H.

In certain embodiments, —R² and —R^{2a} of formula (IIb′) are independently selected from the group consisting of —H, methyl and ethyl. In certain embodiments, at least one of —R² and —R^{2a} of formula (IIb′) is —H. In certain embodiments, both —R² and —R^{2a} of formula (IIb′) are H.

In certain embodiments, —R³ and —R^{3a} of formula (IIb′) are independently selected from the group consisting of —H, methyl, ethyl, propyl and butyl. In certain embodiments, at least one of —R³ and —R^{3a} of formula (IIb′) is methyl. In certain embodiments, —R³ and —R^{3a} of formula (IIb′) are both —H. In certain embodiments, —R³ and —R^{3a} of formula (IIb′) are both methyl. In certain embodiments, —R³ of formula (IIb′) is —H and —R^{3a} of formula (IIb′) is methyl.

In certain embodiments, the moiety -L¹- is of formula (IIc):

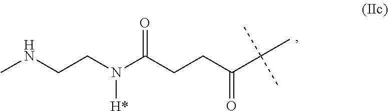

wherein the dashed line indicates the attachment to a nitrogen of -D which is a CNP moiety by forming an amide bond; and
wherein -L¹- is substituted with -L²-Z and wherein -L¹- is optionally further substituted, provided that the hydrogen marked with the asterisk in formula (IIc) is not replaced by -L²-Z or a substituent.

In certain embodiments, -L¹- of formula (IIc) is substituted with one moiety -L²-Z.

In certain embodiments, the moiety -L¹- of formula (IIc) is not further substituted.

In certain embodiments, the moiety -L¹- is of formula (IIc-a):

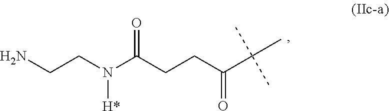

wherein the dashed line indicates the attachment to a nitrogen of -D which is a CNP moiety by forming an amide bond; and
wherein -L¹- is substituted with -L²-Z and wherein -L¹- is optionally further substituted, provided that the hydrogen marked with the asterisk in formula (IIc-a) is not replaced by -L²-Z or a substituent.

In certain embodiments, -L¹- of formula (IIc-a) is substituted with one moiety -L²-Z.

In certain embodiments, the moiety -L¹- of formula (IIc-a) is not further substituted.

In certain embodiments, the moiety -L¹- is of formula (IIc-b):

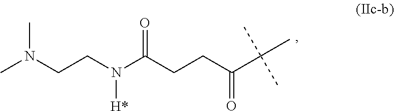

wherein the dashed line indicates the attachment to a nitrogen of -D which is a CNP moiety by forming an amide bond; and
wherein -L¹- is substituted with -L²-Z and wherein -L¹- is optionally further substituted, provided that the hydrogen marked with the asterisk in formula (IIc-b) is not replaced by -L²-Z or a substituent.

In certain embodiments, -L¹- of formula (IIc-b) is substituted with one moiety -L²-Z.

In certain embodiments, the moiety -L¹- of formula (IIc-b) is not further substituted.

In certain embodiments, the moiety -L¹- is selected from the group consisting of formula (IIc-i), (IIc-ii), (IIc-iii), (IIc-iv) and (IIc-v):

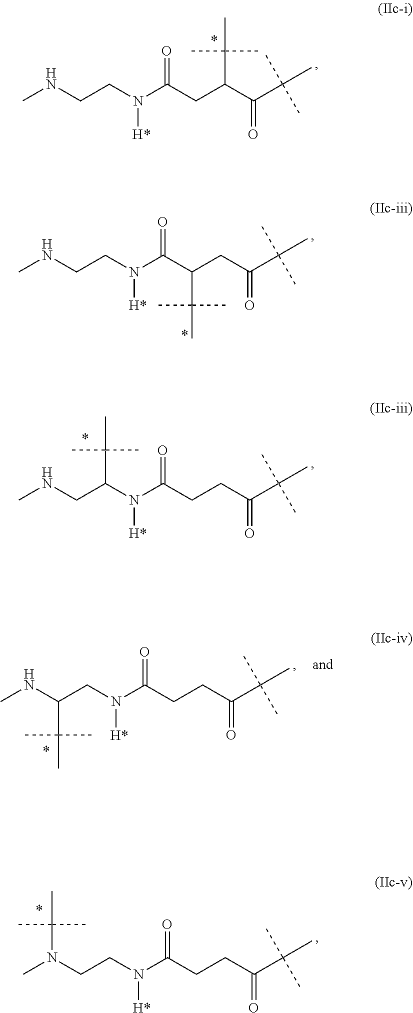

wherein
the unmarked dashed line indicates the attachment to a nitrogen of -D which is a CNP moiety by forming an amide bond; and the dashed line marked with the asterisk indicates attachment to -L²-Z; and
-L¹- is optionally further substituted, provided that the hydrogen marked with the asterisk in formula (IIc-i), (IIc-ii), (IIc-iii), (IIc-iv) and (IIc-v) is not replaced by a substituent.

In certain embodiments, the moiety -L¹- of formula (IIc-i), (IIc-ii), (IIc-iii), (IIc-iv) and (IIc-v) is not further substituted.

In certain embodiments, the moiety -L¹- is of formula (IIc-ii):

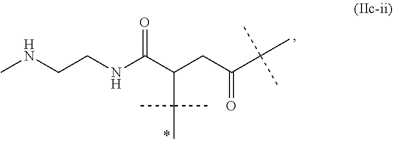

wherein
the unmarked dashed line indicates the attachment to a nitrogen of -D which is a CNP moiety by forming an amide bond; and
the dashed line marked with the asterisk indicates attachment to -L²-Z.

In certain embodiments, -L¹- of formula (IIc-ii) is substituted with one moiety -L²-Z.

In certain embodiments, the moiety -L¹- is selected from the group consisting of formula (IIc-i′), (IIc-ii′), (IIc-iii′), (IIc-iv′) and (IIc-v′):

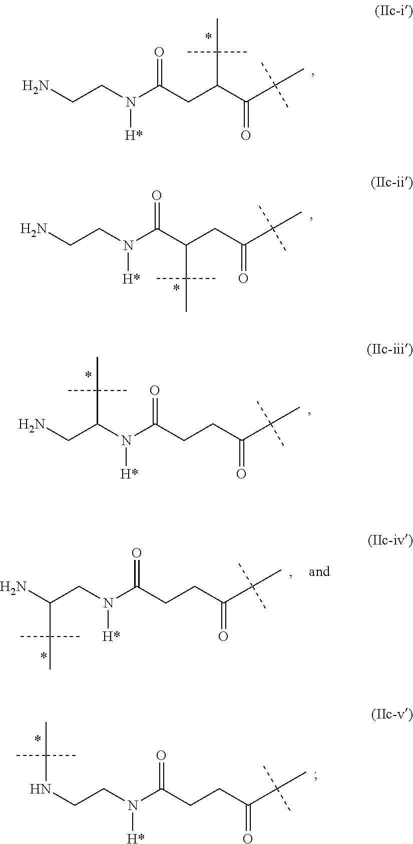

wherein
the unmarked dashed line indicates the attachment to a nitrogen of -D which is a CNP moiety by forming an amide bond;
the dashed line marked with the asterisk indicates attachment to -L²-Z; and
-L¹- is optionally further substituted, provided that the hydrogen marked with the asterisk in formula (IIc-i′), (IIc-ii′), (IIc-iii′), (IIc-iv′) and (IIc-v′) is not replaced by a substituent.

In certain embodiments, the moiety -L¹- of formula (IIc-i′), (IIc-ii′), (IIc-iii′), (IIc-iv′) and (IIc-v′) is not further substituted.

In certain embodiments, the moiety -L¹- is of formula (IIc-ii′):

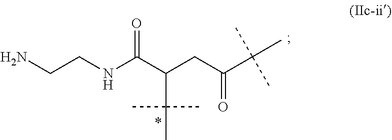

wherein
the unmarked dashed line indicates the attachment to a nitrogen of -D which is a CNP moiety by forming an amide bond; and
the dashed line marked with the asterisk indicates attachment to -L²-Z.

In certain embodiments, -L¹- of formula (IIc-ii′) is substituted with one moiety -L²-Z.

In certain embodiments, the moiety -L¹- is selected from the group consisting of formula (IIc-i″), (IIc-ii″), (IIc-iii″) and (IIc-iv″):

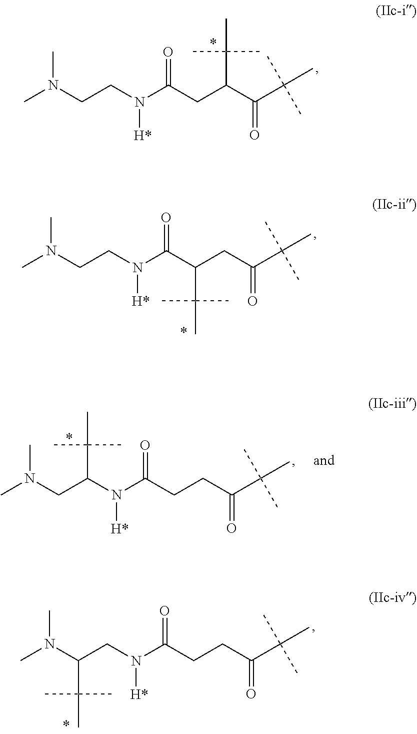

wherein
the unmarked dashed line indicates the attachment to a nitrogen of -D which is a CNP moiety by forming an amide bond;
the dashed line marked with the asterisk indicates attachment to -L²-Z; and
-L¹- is optionally further substituted, provided that the hydrogen marked with the asterisk in formula (IIc-i″), (IIc-ii″), (II-iii″) and (IIc-iv″) is not replaced by a substituent.

In certain embodiments, the moiety -L¹- of formula (IIc-i″), (IIc-ii″), (IIc-iii″) and (IIc-iv″) is not further substituted.

In certain embodiments, the moiety -L¹- is of formula (IIc-ii″):

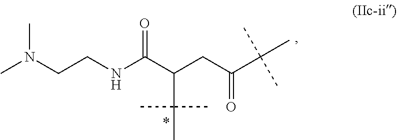

wherein
the unmarked dashed line indicates the attachment to a nitrogen of -D which is a CNP moiety by forming an amide bond; and
the dashed line marked with the asterisk indicates attachment to -L²-Z.

In certain embodiments, -L¹- of formula (IIc-ii″) is substituted with one moiety -L²-Z.

The optional further substituents of -L¹- of formula (II), (IIa), (IIb), (IIb′), (IIc), (IIc-a), (IIc-b), (IIc-i), (IIc-ii), (IIc-iii), (IIc-iv), (IIc-v), (IIc-i′), (IIc-ii′), (IIc-iii′), (IIc-iv′), (IIc-v′), (IIc-i″), (IIc-ii″), (IIc-iii″) and (IIc-iv″) are in certain embodiments as described above.

Another moiety -L¹- is disclosed in WO 2016/020373 A1. Accordingly, in certain embodiments, the moiety -L¹- is of formula (III):

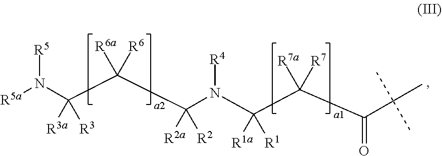

wherein
the dashed line indicates attachment to a primary or secondary amine or hydroxyl of -D which is a CNP moiety by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C≡N, —C(═NR⁸)R^{8a}, —CR⁸(—CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, —Rob are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O) OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein-T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein-T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O) OR^{13a}, N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}—R⁶/—R^{6a}—R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joined together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with -L²-Z and wherein -L¹- is optionally further substituted;
wherein
-L²- is a single chemical bond or a spacer; and
—Z is a carrier moiety, preferably a water-soluble polymeric moiety.

The optional further substituents of -L¹- of formula (III) are in certain embodiments as described above.

In certain embodiments, -L¹- of formula (III) is substituted with one moiety -L²-Z.

In certain embodiments, -L¹- of formula (III) is not further substituted.

Additional embodiments for -L¹- are disclosed in EP1536334B1, WO 2009/009712 A1, WO 2008/034122 A1, WO 2009/143412 A2, WO 2011/082368 A2, and U.S. Pat. No. 8,618,124 B2, which are herewith incorporated by reference in their entirety.

Additional embodiments for -L¹- are disclosed in U.S. Pat. Nos. 8,946,405B2 and 8,754,190B2, which are herewith incorporated by reference in their entirety. Accordingly, a moiety -L¹- is of formula (IV):

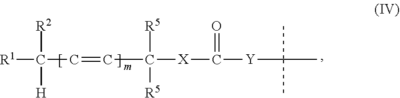

wherein
the dashed line indicates attachment to -D which is a CNP moiety and wherein attachment is through a functional group of -D selected from the group consisting of —OH, —SH and —NH₂;
m is 0 or 1; m
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q) CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3- to 8-membered ring; and
optionally, both —R⁹ together with the nitrogen to which they are attached form a heterocyclic ring;
wherein -L¹- is substituted with -L²-Z and wherein -L¹- is optionally further substituted;
wherein
-L²- is a single chemical bond or a spacer; and
—Z is a carrier moiety, preferably a water-soluble polymeric moiety.

The optional further substituents of -L¹- of formula (IV) are in certain embodiments, as described above.

In certain embodiments, -L¹- of formula (IV) is substituted with one moiety -L²-Z. In certain embodiments, -L¹- of formula (IV) is not further substituted.

Only in the context of formula (IV) the terms used have the following meaning:

The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, in certain embodiments, 6 to 10 carbons, including groups such as phenyl, naphthyl, and anthracenyl. The term “heteroaryl” includes aromatic rings comprising 3 to 15 carbons containing at least one N, O or S atom, in certain embodiments, 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In certain embodiments, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4- to 8-membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

An additional embodiment for -L¹- is disclosed in WO 2013/036857 A1, which is herewith incorporated by reference in its entirety. Accordingly, in certain embodiments, moiety -L¹- is of formula (V):

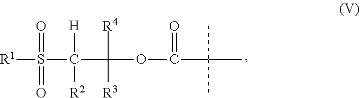

wherein
the dashed line indicates attachment to -D which is a CNP moiety and wherein attachment is through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two-R⁵ can be cycloalkyl or cycloheteroalkyl;
wherein -L¹- is substituted with -L²-Z and wherein -L¹- is optionally further substituted;
wherein
-L²- is a single chemical bond or a spacer; and
—Z is a carrier moiety, preferably a water-soluble polymeric moiety.

The optional further substituents of -L¹- of formula (V) are, in certain embodiments as described above.

In certain embodiments, -L¹- of formula (V) is substituted with one moiety -L²-Z. In certain embodiments, -L¹- of formula (V) is not further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 carbons.

“Aryl” includes aromatic hydrocarbon groups of 6-18 carbons, in certain embodiments, 6-10 carbons, including groups such as phenyl, naphthyl, and anthracene “Heteroaryl” includes aromatic rings comprising 3-15 carbons containing at least one N, O or S atom, in certain embodiments, 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

Another embodiment for -L¹- is disclosed in WO 2022/115563 A1, which is herewith incorporated by reference in its entirety. Accordingly, in certain embodiments, -L¹- is of formula (Va):

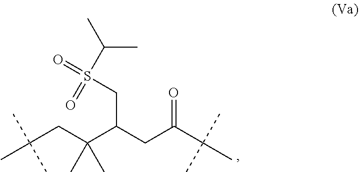

wherein the dashed line marked with the asterisk indicates the attachment to -L²-Z and the unmarked dashed line indicates the attachment to -D.

In certain embodiments, -L¹- is of formula (Va), the dashed line marked with the asterisk indicates the attachment to -L²-Z and the unmarked dashed line indicates the attachment to -D, wherein -D is a CNP moiety of the following amino acid sequence:

SEQ ID NO: 106:
LQEHPQARKYKGAQKKGLSKGCFGLKLDRIGSMSGLGC,

wherein the cysteines at position 22 and 38 are connected through a disulfide-bridge; and
wherein the attachment to -L¹-takes place either at the N-terminus or ring of the peptide.

Another embodiment for -L¹- is disclosed in U.S. Pat. No. 7,585,837 B2, which is herewith incorporated by reference in its entirety. Accordingly, in certain embodiments, a moiety -L¹- is of formula (VI):

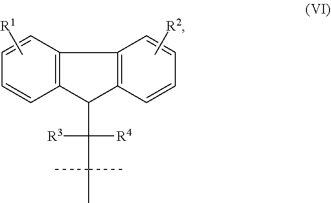

wherein
the dashed line indicates attachment to -D which is a CNP moiety and wherein attachment is through an amine functional group of -D;

R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with -L²-Z and wherein -L¹- is optionally further substituted;
wherein
-L²- is a single chemical bond or a spacer; and
—Z is a carrier moiety, preferably a water-soluble polymeric moiety.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

In certain embodiments, -L¹- of formula (VI) is substituted with one moiety -L²-Z.

The optional further substituents of -L¹- of formula (VI) are in certain embodiments as described above.

In certain embodiments, -L¹- of formula (VI) is not further substituted.

Only in the context of formula (VI) the terms used have the following meaning:
The terms “alkyl”, “alkoxy”, “alkoxyalkyl”, “aryl”, “alkaryl” and “aralkyl” mean alkyl radicals of 1-8, in certain embodiments, 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

A further embodiment for -L¹- is disclosed in WO 2002/089789 A1, which is herewith incorporated by reference in its entirety. Accordingly, a moiety -L¹- is of formula (VII):

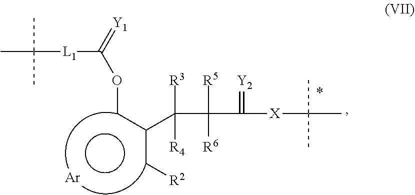

wherein
the dashed line indicates attachment to -D which is a CNP moiety and wherein attachment is through an amine functional group of -D;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with -L²-Z and wherein -L¹- is optionally further substituted;
wherein
-L²- is a single chemical bond or a spacer; and
—Z is a carrier moiety, preferably a water-soluble polymeric moiety.

In certain embodiments, -L¹- of formula (VII) is substituted with one moiety -L²-Z.

The optional further substituents of -L¹- of formula (VII) are in certain embodiments, as described above.

In certain embodiments, -L¹- of formula (VII) is not further substituted.

Only in the context of formula (VII) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moieities such as methoxy; and phenoxy includes moieties such as 3-nitrophenoxy. Halo-shall be understood to include fluoro, chloro, iodo and bromo.

In certain embodiments, -L¹-comprises a substructure of formula (VIII):

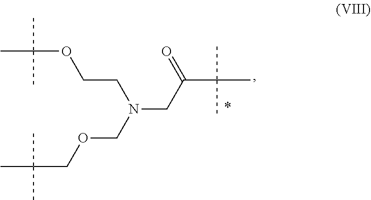

wherein
the dashed line marked with the asterisk indicates attachment to a nitrogen of -D which is a CNP moiety by forming an amide bond;
the unmarked dashed lines indicate attachment to the remainder of -L¹-; and
wherein -L¹- is substituted with -L²-Z and wherein -L¹- is optionally further substituted;
   wherein
   -L²- is a single chemical bond or a spacer; and
   —Z is a carrier moiety, preferably a water-soluble polymeric moiety.

In certain embodiments, -L¹- of formula (VIII) is substituted with one moiety -L²-Z.

The optional further substituents of -L¹- of formula (VIII) are as described above.

In certain embodiments, -L¹- of formula (VIII) is not further substituted.

In certain embodiments, -L¹-comprises a substructure of formula (IX):

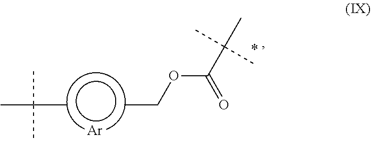

wherein
the dashed line marked with the asterisk indicates attachment to a nitrogen of -D which is a CNP moiety by forming a carbamate bond;
the unmarked dashed lines indicate attachment to the remainder of -L¹-; and wherein -L¹- is substituted with -L²-Z and wherein -L¹- is optionally further substituted;
   wherein
   -L²- is a single chemical bond or a spacer; and
   —Z is a carrier moiety, preferably a water-soluble polymeric moiety.

The optional further substituents of -L¹- of formula (IX) are as described above.

In certain embodiments, -L¹- of formula (IX) is substituted with one moiety -L²-Z.

In certain embodiments, -L¹- of formula (IX) is not further substituted.

The moiety-D may be connected to -L¹-through any functional group of D-H and is connected to -L¹- through an amine functional group of D-H. This may be the N-terminal amine functional group or an amine functional group provided by a lysine side chain, i.e. by the lysines at position 9, 11, 15, 16, 20 and 26, if the CNP has the sequence of SEQ ID NO: 24.

Attachment of -L¹- to the ring of a CNP moiety significantly reduces the CNP conjugate's affinity to NPR-B compared to attachment at the N-terminus or to the non-ring part of CNP, which reduced affinity to NPR-B in turn reduces the risk of cardiovascular side effects, such as hypotension.

Accordingly, in certain embodiments, -L¹- is conjugated to the side chain of an amino acid residue of said ring moiety of -D or to the backbone of said ring moiety of -D. In certain embodiments, -L¹- is covalently and reversibly conjugated to the side chain of an amino acid residue of said ring moiety of -D. If -D is a CNP moiety with the sequence of SEQ ID NO: 24, -L¹- is, in certain embodiments, conjugated to the amine functional group provided by the lysine at position 26 of the corresponding drug D-H.

The moiety -L²- is a chemical bond or a spacer moiety. In certain embodiments, -L²- is a chemical bond. In certain embodiments, -L²- is a spacer moiety.

The moiety -L²- can be attached to -L¹- by replacing any —H present, except where explicitly excluded.

When-L²- is other than a single chemical bond, -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein-T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein-T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O) OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

When -L²- is other than a single chemical bond, -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₂₀ alkyl, C₂₋₂₀ alkenyl, and C₂₋₂₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein-T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, OXO (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(RYSR^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O) OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

When -L²- is other than a single chemical bond, -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl;
wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; each —R^{y2} is independently selected from the group consisting of halogen, and C₁₋₆ alkyl; and each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments, -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments, -L²- has a molecular weight in the range of from 14 g/mol to 750 g/mol.

In certain embodiments, -L²- has a chain length of 1 to 20 atoms.

As used herein, the term “chain length” with regard to the moiety -L²-refers to the number of atoms of -L²- present in the shortest connection between -L¹- and —Z.

In certain embodiments, -L²- is of formula (i):

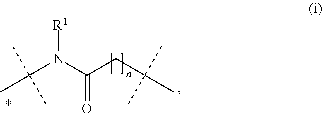

wherein
the dashed line marked with the asterisk indicates attachment to -L¹-;
the unmarked dashed line indicates attachment to —Z;
—R¹ is selected from the group consisting of —H, C₁₋₆ alkyl, C₂₋₆ alkenyl and C₂₋₆ alkynyl;
n is selected from the group consisting of 0, 1, 2, 3, 4, 5, 6, 7, 8, 9, 10, 11, 12, 13, 14, 15, 16, 17 and 18; and
wherein the moiety of formula (i) is optionally further substituted.

In certain embodiments, —R¹ of formula (i) is selected from the group consisting of —H, methyl, ethyl, propyl, and butyl. In certain embodiments, —R¹ of formula (i) is selected from the group consisting of —H, methyl, ethyl and propyl. In certain embodiments, —R¹ of formula (i) is selected from the group consisting of —H and methyl. In certain embodiments, —R¹ of formula (i) is methyl.

In certain embodiments, n of formula (i) is selected from the group consisting of 0, 1, 2, 3, 4, 5, 6, 7, 8, 9 and 10. In certain embodiments, n of formula (i) is selected from the group consisting of 0, 1, 2, 3, 4 and 5. In certain embodiments, n of formula (i) is selected from the group consisting of 0, 1, 2 and 3. In certain embodiments, n of formula (i) is selected from the group consisting of 0 and 1. In certain embodiments, n of formula (i) is 0.

In certain embodiments, -L²- is a moiety selected from the group consisting of

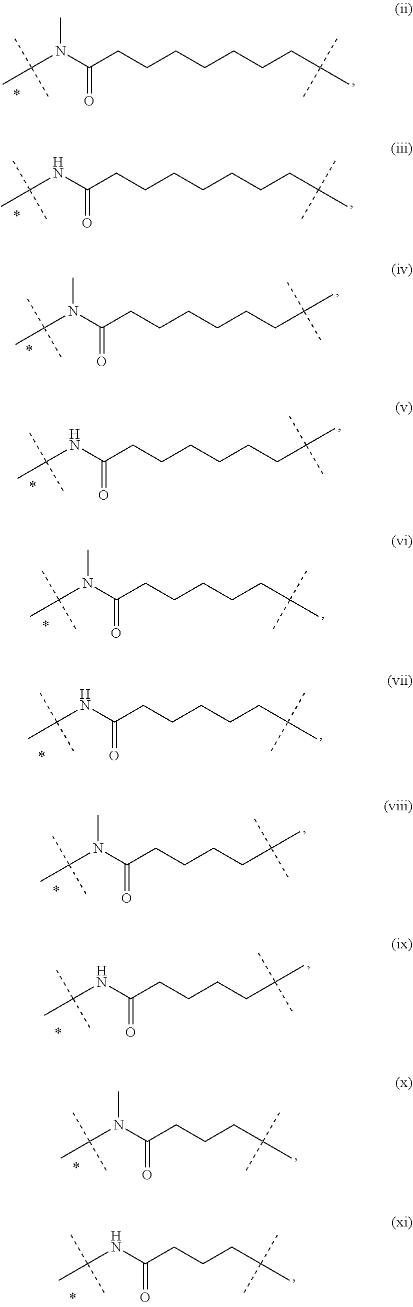

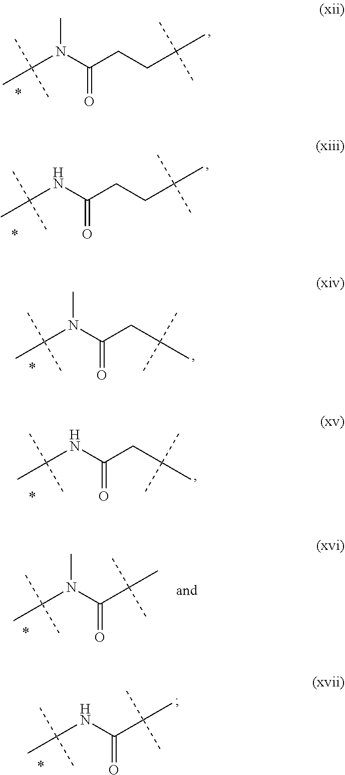

wherein
the dashed line marked with the asterisk indicates attachment to -L¹-;
the unmarked dashed line indicates attachment to —Z and
wherein the moieties (ii), (iii), (iv), (v), (vi), (vii), (viii), (ix), (x), (xi), (xii), (xiii), (xiv), (xv), (xvi) and (xvii) are optionally further substituted.

In certain embodiments, -L²- is selected from the group consisting of

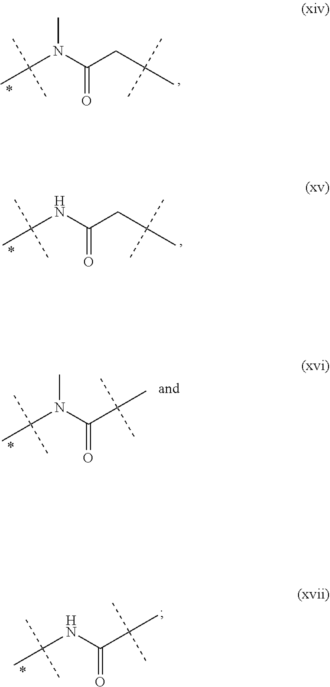

wherein
the dashed line marked with the asterisk indicates attachment to -L¹-; and
the unmarked dashed line indicates attachment to —Z.

In certain embodiments, -L²- is selected from the group consisting of

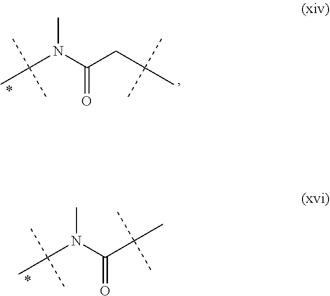

wherein
the dashed line marked with the asterisk indicates attachment to -L¹-; and
the unmarked dashed line indicates attachment to —Z.

In certain embodiments, -L²- is of formula (xvi):

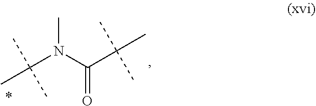

wherein
the dashed line marked with the asterisk indicates attachment to -L¹-; and
the unmarked dashed line indicates attachment to —Z.

In certain embodiments, the moiety -L¹-L²- is selected from the group consisting of:

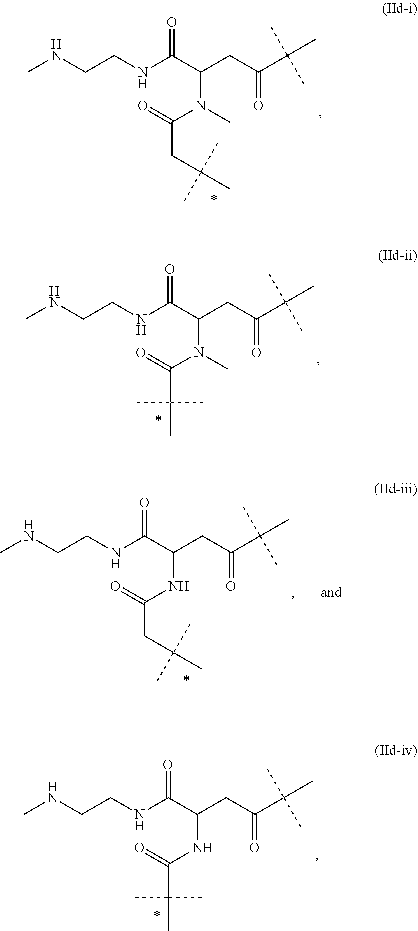

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is of formula (IId-ii):

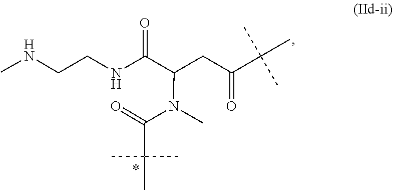

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is of formula (IId-ii′):

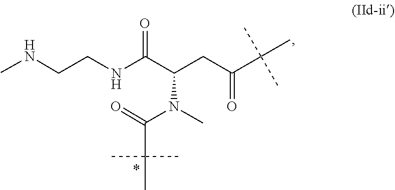

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is selected from the group consisting of:

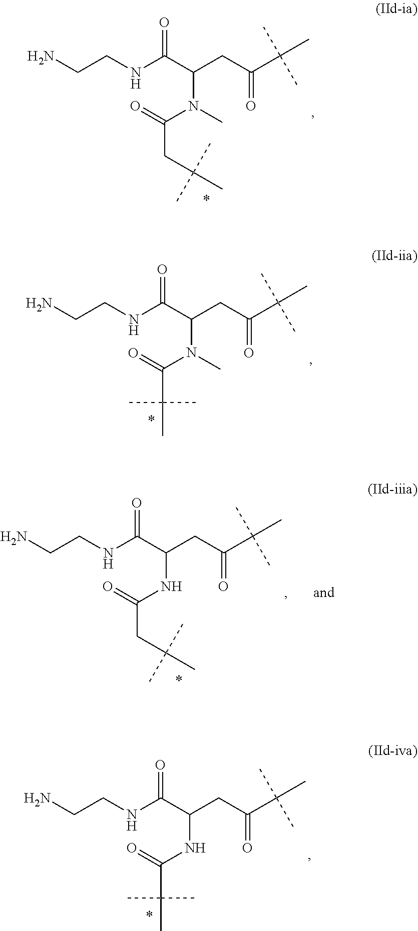

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is of formula (IId-iia):

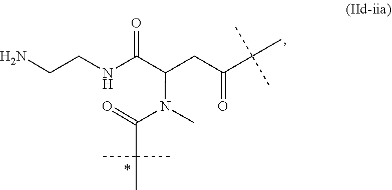

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is of formula (IId-iia′):

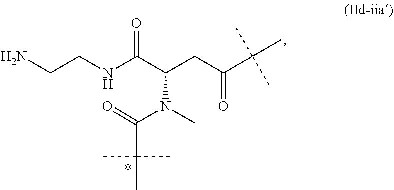

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is selected from the group consisting of:

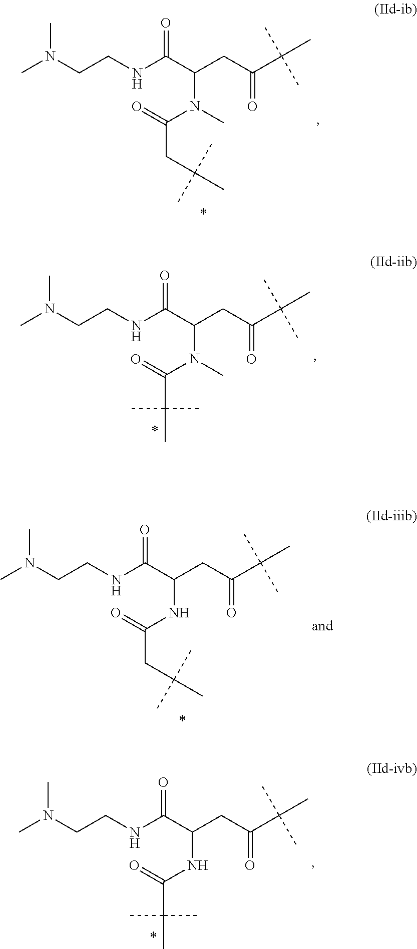

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is of formula (IId-iib):

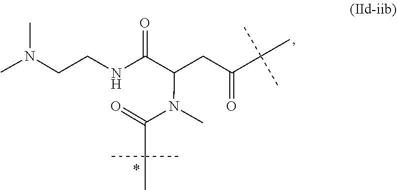

wherein the unmarked dashed line indicates the attachment to a nitrogen of -D which is a CNP moiety by forming an amide bond; and the dashed line marked with the asterisk indicates attachment to —Z.

In certain embodiments, the moiety -L¹-L²- is of formula (IId-iib′):

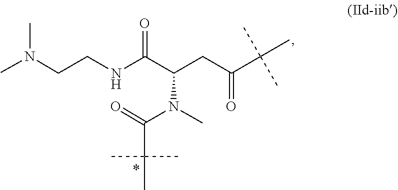

wherein
the unmarked dashed line indicates the attachment to a nitrogen of -D which is a CNP moiety by forming an amide bond; and
the dashed line marked with the asterisk indicates attachment to —Z.

In certain embodiments, —Z of formula (Ia) or (Ib) has a molecular weight ranging from 5 to 200 kDa. In certain embodiments, —Z of formula (Ia) or (Ib) has a molecular weight ranging from 8 to 100 kDa. In certain embodiments, —Z of formula (Ia) or (Ib) has a molecular weight ranging from 10 to 80 kDa. In certain embodiments, —Z of formula (Ia) or (Ib) has a molecular weight ranging from 12 to 60 kDa. In certain embodiments, —Z of formula (Ia) or (Ib) has a molecular weight ranging from 15 to 40 kDa. In certain embodiments, —Z of formula (Ia) or (Ib) has a molecular weight of about 20 kDa. In certain embodiments, —Z of formula (Ia) or (Ib) has a molecular weight of about 40 kDa.

The polymeric moiety —Z of formula (Ia) or (Ib) comprises a polymer. In certain embodiments, —Z of formula (Ia) or (Ib) comprises a polymer selected from the group consisting of poly(2-methacryloyl-oxyethyl phosphoryl cholines), poly(acrylic acids), poly(acrylamides), poly(alkyloxy) polymers, poly(amides), poly(acrylates), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, —Z of formula (Ia) or (Ib) comprises a protein. Preferred proteins are selected from the group consisting of carboxyl-terminal peptide of the chorionic gonadotropin as described in US 2012/0035101 A1 which are herewith incorporated by reference; albumin; XTEN™ sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc-fusion proteins.

In certain embodiments, —Z of formula (Ia) or (Ib) is a polysarcosine. In certain embodiments, —Z of formula (Ia) or (Ib) comprises poly(N-methylglycine). In certain embodiments, —Z of formula (Ia) or (Ib) comprises a random coil protein moiety. In certain embodiments, —Z of formula (Ia) or (Ib) comprises one random coil protein moiety. In certain embodiments, —Z of formula (Ia) or (Ib) comprises two random coil protein moieties. In certain embodiments, —Z of formula (Ia) or (Ib) comprises three random coil protein moieties. In certain embodiments, —Z of formula (Ia) or (Ib) comprises four random coil protein moieties. In certain embodiments, —Z of formula (Ia) or (Ib) comprises five random coil protein moieties. In certain embodiments, —Z of formula (Ia) or (Ib) comprises six random coil protein moieties. In certain embodiments, —Z of formula (Ia) or (Ib) comprises seven random coil protein moieties. In certain embodiments, —Z of formula (Ia) or (Ib) comprises eight random coil protein moieties.

In certain embodiments, such random coil protein moiety comprises at least 25 amino acid residues and at most 2000 amino acids. In certain embodiments, such random coil protein moiety comprises at least 30 amino acid residues and at most 1500 amino acid residues. In certain embodiments, such random coil protein moiety comprises at least 50 amino acid residues and at most 500 amino acid residues.

In certain embodiments, —Z of formula (Ia) or (Ib) comprises a fatty acid derivative. In certain embodiments, —Z of formula (Ia) or (Ib) is a fatty acid derivative. In certain embodiments, —Z of formula (Ia) is a fatty acid derivative and x is 1.

In certain embodiments, —Z of formula (Ia) or (Ib) is a fatty acid derivative as disclosed in WO 2006/097537 A2 which is herewith incorporated by reference.

In certain embodiments, —Z of formula (Ia) or (Ib) comprises a fatty acid derivative as disclosed in WO 2021/055497 A1 which is herewith incorporated by reference. Accordingly, in certain embodiments, —Z of formula (Ia) or (Ib) has the following structure (w):

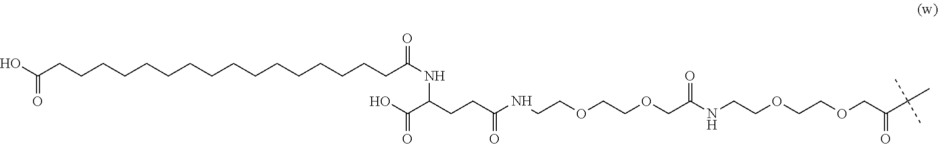

wherein the dashed line indicates the attachment to -L²- or -L¹- in formula (Ia) or (Ib).

In certain embodiments, —Z is of formula (w) and -L¹- is of formula (V).

In certain embodiments, —Z-L²-L¹- is of formula (w-a):

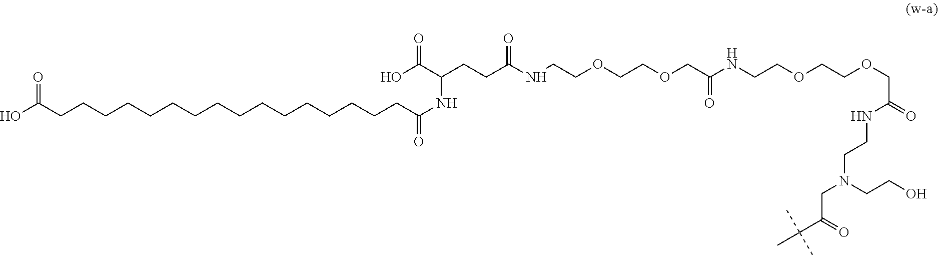

wherein the dashed line indicates the attachment to -D of formula (Ia) or (Ib).

In certain embodiments, CNP has the sequence selected from the group consisting of:

(SEQ ID NO: 98)
PGQEHPQARRYRGAQRRGLSRGCFGLKLDRIGSMSGLGC;
(SEQ ID NO: 30)
PGQEHPNARKYKGANKKGLSKGCFGLKLDRIGSMSGLGC;
(SEQ ID NO: 99)
PGQEHPNARRYRGANRRGLSRGCFGLKLDRIGSMSGLGC;
and
(SEQ ID NO: 100)
PGQEHPQARKYKGAQKKGLSKGCFGLKLDRIGSMSGLGC.

In certain embodiments, CNP has the sequence selected from the group consisting of SEQ ID NO: 98, SEQ ID NO:30, SEQ ID NO:99 and SEQ ID NO: 100, —Z is of formula (w) and -L¹- is a reversible linker moiety. In certain embodiments, CNP has the sequence selected from the group consisting of SEQ ID NO:98, SEQ ID NO:30, SEQ ID NO:99 and SEQ ID NO: 100, —Z is of formula (w) and -L¹- is of formula (V). In certain embodiments, CNP has the sequence selected from the group consisting of SEQ ID NO:98, SEQ ID NO:30, SEQ ID NO:99 and SEQ ID NO:100, —Z-L²-L¹- is of formula (w-a). Said -L¹- may be attached to said CNP via a lysine other than the lysine within the ring structure or it may be attached to the N-terminus.

In certain embodiments, CNP of SEQ ID NO:98, SEQ ID NO:30, SEQ ID NO:99 and SEQ ID NO: 100 further comprises an acetyl group, such as an acetyl group at the N-terminus of the peptide. In certain embodiments, CNP of SEQ ID NO:98, SEQ ID NO:30, SEQ ID NO:99 and SEQ ID NO:100 further comprises an —OH or —NH₂ group at the C-terminus. In certain embodiments, CNP of SEQ ID NO:98, SEQ ID NO:30, SEQ ID NO:99 and SEQ ID NO:100 and -L¹- is attached to a residue of the CNP ring moiety or at a site other than the CNP moiety.

In certain embodiments, -L¹- is attached at a lysine residue, such as the lysine residue in bold in SEQ ID NO:98, SEQ ID NO:30, SEQ ID NO:99 and SEQ ID NO:100:

(SEQ ID NO: 98)
PGQEHPQARRYRGAQRRGLSRGCFGLKLDRIGSMSGLGC;
(SEQ ID NO: 30)
PGQEHPNARKYKGANKKGLSKGCFGLKLDRIGSMSGLGC;
(SEQ ID NO: 99)
PGQEHPNARRYRGANRRGLSRGCFGLKLDRIGSMSGLGC;
and
(SEQ ID NO: 100)
PGQEHPQARKYKGAQKKGLSKGCFGLKLDRIGSMSGLGC.

In certain embodiments, CNP is selected from the group consisting of:

(SEQ ID NO: 101)
Ac-PGQEHPQARRYRGAQRRGLSRGCFGLKLDRIGSMSGLGC;
(SEQ ID NO: 102)
Ac-PGQEHPNARKYKGANKKGLSKGCFGLKLDRIGSMSGLGC-NH₂;
(SEQ ID NO: 103)
Ac-PGQEHPNARRYRGANRRGLSRGCFGLKLDRIGSMSGLGC;
(SEQ ID NO: 104)
Ac-PGQEHPNARRYRGANRRGLSRGCFGLKLDRIGSMSGLGC-NH₂;
and
(SEQ ID NO: 105)
Ac-PGQEHPQARRYRGAQRRGLSRGCFGLKLDRIGSMSGLGC-NH₂.

In certain embodiments, —Z of formula (Ia) or (Ib) is a hyaluronic acid-based polymer. In certain embodiments, —Z of formula (Ia) or (Ib) is a polymeric moiety as disclosed in WO 2013/024047 A1 which is herewith incorporated by reference.

In certain embodiments, —Z of formula (Ia) or (Ib) is a polymeric moiety as disclosed in WO 2013/024048 A1 which is herewith incorporated by reference.

In certain embodiments, —Z of formula (Ia) or (Ib) is a PEG-based polymer. In certain embodiments, —Z is a branched or multi-arm PEG-based polymer.

In certain embodiments, —Z of formula (Ia) or (Ib) is a branched polymer. In certain embodiments, —Z of formula (Ia) or (Ib) is a branched polymer having one, two, three, four, five or six branching points. In certain embodiments, —Z of formula (Ia) or (Ib) is a branched polymer having one, two or three branching points. In certain embodiments, —Z of formula (Ia) or (Ib) is a branched polymer having one branching point. In certain embodiments, —Z of formula (Ia) or (Ib) is a branched polymer having two branching points. In certain embodiments, —Z of formula (Ia) or (Ib) is a branched polymer having three branching points.

In certain embodiments, a branching point is selected from the group consisting of —N<, —CH< and >C<.

In certain embodiments, such branched moiety —Z of formula (Ia) or (Ib) is PEG-based.

In certain embodiments, such branched moiety —Z of formula (Ia) or (Ib) has a molecular weight ranging from and including 5 kDa to 500 kDa. In certain embodiments, such branched moiety —Z of formula (Ia) or (Ib) has a molecular weight ranging from and including 10 kDa to 250 kDa. In certain embodiments, such branched moiety —Z of formula (Ia) or (Ib) has a molecular weight ranging from and including 10 kDa to 150 kDa. In certain embodiments, such branched moiety —Z of formula (Ia) or (Ib) has a molecular weight ranging from and including 12 kDa to 100 kDa. In certain embodiments, such branched moiety —Z of formula (Ia) or (Ib) has a molecular weight ranging from and including 15 kDa to 80 kDa. In certain embodiments, such branched moiety —Z of formula (Ia) or (Ib) has a molecular weight ranging from and including 10 kDa to 80 kDa. In certain embodiments, the molecular weight is about 10 kDa. In certain embodiments, the molecular weight of such branched moiety —Z of formula (Ia) or (Ib) is about 20 kDa. In certain embodiments, the molecular weight of such branched moiety —Z of formula (Ia) or (Ib) is about 30 kDa. In certain embodiments, the molecular weight of such a branched moiety —Z of formula (Ia) or (Ib) is about 40 kDa. In certain embodiments, the molecular weight of such a branched moiety —Z of formula (Ia) or (Ib) is about 50 kDa. In certain embodiments, the molecular weight of such a branched moiety —Z of formula (Ia) or (Ib) is about 60 kDa. In certain embodiments, the molecular weight of such a branched moiety —Z of formula (Ia) or (Ib) is about 70 kDa. In certain embodiments, the molecular weight of such a branched moiety —Z of formula (Ia) or (Ib) is about 80 kDa. In certain embodiments, such branched moiety —Z of formula (Ia) or (Ib) has a molecular weight of about 40 kDa.

In certain embodiments, —Z comprises a moiety

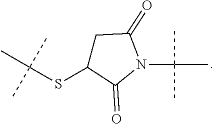

In certain embodiments, —Z comprises an amide bond.

In certain embodiments, —Z of formula (Ia) or (Ib) comprises a moiety of formula (a)

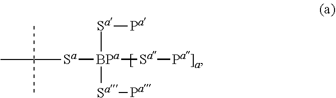

wherein
the dashed line indicates attachment to -L²- or to the remainder of —Z;
BP^{a} is a branching point selected from the group consisting of —N<, —CR< and >C<;
—R is selected from the group consisting of —H and C₁₋₆ alkyl;
a is 0 if BP^{a} is —N< or —CR< and a is 1 if BP^{a} is >C<;
S^{a}—, —S^{a′}—, —S^{a″}— and —S^{a′″}— are independently of each other a chemical bond or are selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O) OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different; and
-P^{a′}, -P^{a″} and -P^{a′″} are independently a polymeric moiety.

Optionally, the moiety of formula (a) is substituted with one or more substituents.

In certain embodiments, BP^{a} of formula (a) is —N<. In certain embodiments, BP^{a} of formula (a) is —CR<. In certain embodiments, —R is —H. Accordingly, in certain embodiments, a of formula (a) is 0. In certain embodiments, BP^{a} of formula (a) is >C<.

In certain embodiments, —S^{a}— of formula (a) is a chemical bond.

In certain embodiments, —S^{a}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments, —S^{a}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —S^{a′}— of formula (a) is a chemical bond.

In certain embodiments, —S^{a′}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments, —S^{a′}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —S^{a″}— of formula (a) is a chemical bond.

In certain embodiments, —S^{a″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments, —S^{a″}— of formula (a) is selected from the group consisting of methyl, ethyl, propyl, butyl, which are optionally interrupted by one or more chemical groups selected from the group consisting of —O—, —C(O)— and —C(O)N(R⁴)—. In certain embodiments, —S^{a′″}— of formula (a) is a chemical bond.

In certain embodiments, —S^{a′″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments, —S^{a′″}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, -P^{a′}, -P^{a″} and -P^{a′″} of formula (a) independently comprise a polymer selected from the group consisting of poly(2-methacryloyl-oxyethyl phosphoryl cholines), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, -P^{a′}, -P^{a″} and -P^{a′″} of formula (a) independently have a molecular weight ranging from and including 5 kDa to 50 kDa, in certain embodiments ranging from and including 5 kDa to 40 kDa, in certain embodiments ranging from and including 7.5 kDa to 35 kDa, in certain embodiments ranging from and 7.5 to 30 kDa, in certain embodiments ranging from and including 10 to 30 kDa.

In certain embodiments, -P^{a′}, -P^{a″} and -P^{a′″} of formula (a) have a molecular weight of about 5 kDa. In certain embodiments, -P^{a′}, -P^{a″} and -P^{a′″} of formula (a) have a molecular weight of about 7.5 kDa. In certain embodiments, -P^{a′}, -P^{a″} and -P^{a′″} of formula (a) have a molecular weight of about 10 kDa. In certain embodiments, -P^{a′}, -P^{a″} and -P^{a′″} of formula (a) have a molecular weight of about 12.5 kDa. In certain embodiments, -P^{a′}, -P^{a″} and -P^{a′″} of formula (a) have a molecular weight of about 15 kDa. In certain embodiments, -P^{a′}, -P^{a″} and -P^{a′″} of formula (a) have a molecular weight of about 20 kDa.

In certain embodiments, -P^{a′}, -P^{a″} and -P^{a′″} of formula (a) independently comprise a PEG-based moiety. In certain embodiments, -P^{a′}, -P^{a″} and -P^{a′″} of formula (a) independently comprise a PEG-based moiety comprising at least 20% PEG, in certain embodiments at least 30% PEG, in certain embodiments at least 40% PEG, in certain embodiments at least 50% PEG, in certain embodiments at least 60% PEG, in certain embodiments at least 70% PEG, in certain embodiments at least 80% PEG and in certain embodiments at least 90% PEG.

In certain embodiments, -P^{a′}, -P^{a″} and -P^{a′″} of formula (a) independently comprise a protein moiety, in certain embodiments a random coil protein moiety and in certain embodiments a random coil protein moiety selected from the group consisting of PA, PAS, PAG, PG and XTEN™ moieties.

In certain embodiments, -P^{a′}, -P^{a″} and -P^{a′″} of formula (a) are a PA moiety. In certain embodiments, -P^{a′}, -P^{a″} and -P^{a′″} of formula (a) are a PAS moiety. In certain embodiments, -P^{a′}, -P^{a″} and -P^{a′″} of formula (a) are a PAG moiety. In certain embodiments, -P^{a′}, -P^{a″} and -P^{a′″} of formula (a) are a PG moiety. In certain embodiments, -P^{a′}, -P^{a″} and -P^{a′″} of formula (a) are an XTEN™ moiety.

In certain embodiments, —Z comprises one moiety of formula (a). In certain embodiments, —Z comprises two moieties of formula (a). In another embodiment, —Z comprises three moieties of formula (a). In certain embodiments, —Z comprises four moieties of formula (a). In certain embodiments, —Z comprises five moieties of formula (a). In certain embodiments, —Z comprises six moieties of formula (a).

In certain embodiments, —Z comprises a moiety of formula (b):

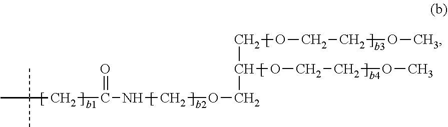

wherein
the dashed line indicates attachment to -L²- or to the remainder of —Z;
b1 is selected from the group consisting of 0, 1, 2, 3, 4, 5, 6, 7 and 8;
b2 is selected from the group consisting of 1, 2, 3, 4, 5, 6, 7 and 8;
b3 is an integer ranging from and including 150 to 1000; in certain embodiments, ranging from and including 150 to 500; and in certain embodiments, ranging from and including 200 to 460; and
b4 is an integer ranging from and including 150 to 1000; in certain embodiments, ranging from and including 150 to 500; and in certain embodiments, ranging from and including 200 to 460.

Optionally, the moiety of formula (b) is substituted with one or more substituents.

In certain embodiments, b3 and b4 of formula (b) are the same integer. In certain embodiments, b3 and b4 of formula (b) are both an integer ranging from 200 to 250 and in certain embodiments, b3 and b4 of formula (b) are about 225. In certain embodiments, b3 and b4 of formula (b) are both an integer ranging from 400 to 500 and in certain embodiments, b3 and b4 of formula (b) are about 450.

In certain embodiments, b1 of formula (b) is selected from the group consisting of 0, 1, 2, 3 and 4. In certain embodiments, b1 of formula (b) is selected from the group consisting of 1, 2 and 3. In certain embodiments, b1 of formula (b) is 2.

In certain embodiments, b2 of formula (b) is selected from the group consisting of 1, 2, 3, 4 and 5. In certain embodiments, b2 of formula (b) is selected from the group consisting of 2, 3 and 4. In certain embodiments, b2 of formula (b) is 3.

In certain embodiments, b1 of formula (b) is 2, b2 of formula (b) is 3, and b3 and b4 are both about 450. In certain embodiments, b1 of formula (b) is 2, b2 of formula (b) is 3, and b3 and b4 are both about 225.

In certain embodiments, —Z comprises one moiety of formula (b). In certain embodiments, —Z comprises two moieties of formula (b). In certain embodiments, —Z comprises three moieties of formula (b). In certain embodiments, —Z comprises four moieties of formula (b). In certain embodiments, —Z comprises five moieties of formula (b). In certain embodiments, —Z comprises six moieties of formula (b).

In certain embodiments, —Z comprises a moiety of formula (c):

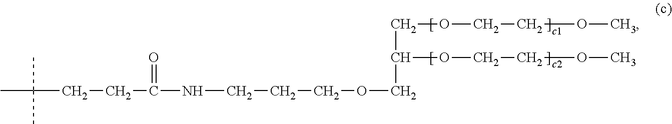

wherein
the dashed line indicates attachment to -L²- or to the remainder of —Z;
c1 and c2 are independently an integer ranging from and including 150 to 500; in certain embodiments, ranging from and including 200 to 460.

Optionally, the moiety of formula (c) is substituted with one or more substituents.

In certain embodiments, both c1 and c2 of formula (c) are the same integer.

In certain embodiments, c1 and c2 of formula (c) range from and include 200 to 250 and in certain embodiments, are about 225. In certain embodiments, c1 and c2 of formula (c) range from and include 400 to 500 and in certain embodiments, c1 and c2 of formula (c) are about 450.

In certain embodiments, the moiety —Z is a branched PEG-based polymer comprising at least 10% PEG, has one branching point and two PEG-based polymer arms and has a molecular weight of about 40 kDa. Accordingly, each of the two PEG-based polymer arms has a molecular weight of about 20 kDa. In certain embodiments, the branching point is —CH<.

In certain embodiments, —Z comprises one moiety of formula (c). In certain embodiments, —Z comprises two moieties of formula (c). In certain embodiments, —Z comprises three moieties of formula (c). In certain embodiments, —Z comprises four moieties of formula (c). In certain embodiments, —Z comprises five moieties of formula (c). In certain embodiments, —Z comprises six moieties of formula (c).

In certain embodiments, the moiety —Z is of formula (d)

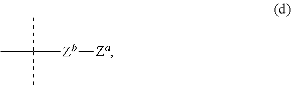

wherein
the dashed line indicates attachment to -L²-;
—Z^{b}— is selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
   each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O) OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different;
and —Z^{a} is

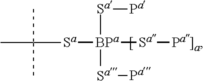

wherein
BP^{a}, —S^{a}—, —S^{a′}, —Sa″—, —S^{a′″}—, -P^{a′}, -P^{a″}, -P^{a′″} and a are used as defined for formula (a).

Optionally, the moiety of formula (d) is substituted with one or more substituents.

In certain embodiments, BP^{a}, —S^{a}—, —S^{a′}—, —S^{a″}—, —S^{a′″}—, —P^{a′}, -P^{a″}, -P^{a′″} of formula (d) are as defined above for formula (a).

In certain embodiments, —Z^{a} of formula (d) is of formula (b). In certain embodiments, b1, b2, b3 and b4 are as described for formula (b).

In certain embodiments, the moiety —Z of formula (Ia) or (Ib) is of formula (e):

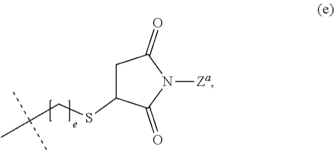

wherein
the dashed line indicates attachment to -L²-;
e is selected from the group consisting of 1, 2, 3, 4, 5, 6, 7, 8, 9, 10, 11, 12, 13, 14 and 15; and —Z^{a} is

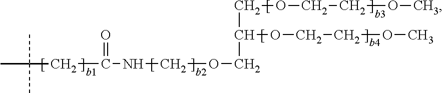

wherein
b1, b2, b3 and b4 are used as defined for formula (b).

Optionally, the moiety of formula (e) is substituted with one or more substituents.

In certain embodiments, for b1, b2, b3 and b4 of formula (e) are as defined above for formula (b).

In certain embodiments, e of formula (e) is 1. In certain embodiments, e of formula (e) is 2. In certain embodiments, e of formula (e) is 3. In certain embodiments, e of formula (e) is 4. In certain embodiments, e of formula (e) is 5. In certain embodiments, e of formula (e) is 6. In certain embodiments, e of formula (e) is 7. In certain embodiments, e of formula (e) is 8. In certain embodiments, e of formula (e) is 9. In certain embodiments, e of formula (e) is 10. In certain embodiments, e of formula (e) is 11. In certain embodiments, e of formula (e) is 12. In certain embodiments, e of formula (e) is 13. In certain embodiments, e of formula (e) is 14. In certain embodiments, e of formula (e) is 15.

In certain embodiments, e of formula (e) is selected from the group consisting of 2, 3, 4, 5, 6, 7, 8 and 9. In certain embodiments, e of formula (e) is selected from 3, 4, 5 and 6. In certain embodiments, e of formula (e) is 5.

In certain embodiments, e of formula (e) is 5, b1 of formula (e) is 2, b2 of formula (e) is 3 and b3 and b4 of formula (e) are both about 450.

In certain embodiments, the moiety —Z of formula (Ia) or (Ib) is of formula (e-i) or (e-i′):

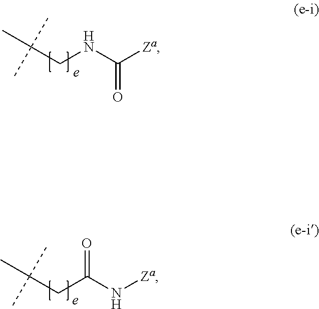

wherein
the dashed line indicates attachment to -L²-;
e is selected from the group consisting of 1, 2, 3, 4, 5, 6, 7, 8, 9, 10, 11, 12, 13, 14 and 15; and
—Z^{a} is

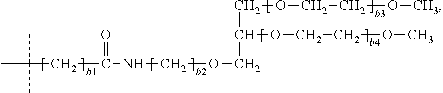

wherein
b1, b2, b3 and b4 are used as defined for formula (b).

In certain embodiments, for b1, b2, b3 and b4 of formula (e-i) and (e-i′) are as defined above for formula (b).

In certain embodiments, e of formula (e-i) and (e-i′) are as described for formula (e).

In certain embodiments, b1 of formula (e-i) and (e-i′) is 2, b2 of formula (e-i) and (e-i′) is 3 and b3 and b4 of formula (e-i) and (e-i′) are both about 450.

In certain embodiments, —Z of formula (Ia) or (Ib) is of formula (e-i).

In certain embodiments, the moiety —Z is a branched PEG-based polymer comprising at least 10% PEG, has three branching points and four PEG-based polymer arms and has a molecular weight of about 40 kDa. Accordingly, each of the four PEG-based polymer arms has a molecular weight of about 10 kDa. In certain embodiments, each of the three branching points is —CH<.

In certain embodiments, the moiety —Z is of formula (f):

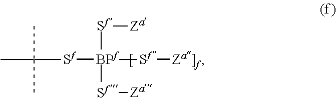

wherein
the dashed line indicates attachment to -L²-;
BP^{f} is a branching point selected from the group consisting of —N<, —CR< and >C<; —R is selected from the group consisting of —H and C₁₋₆ alkyl;
f is 0 if BP^{f} is —N< or —CR< and f is 1 if BP^{f} is >C<;
—S^{f}—, —S^{f′}—, —S^{f″}— and —S^{f′″}— are independently either a chemical bond or are independently selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
   each R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O) OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different;
and —Z^{a′}, —Z^{a″} and —Z^{a′″} are independently

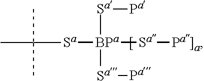

wherein
BP^{a}, —S^{a}—, —S^{a′}—, —S^{a″}—, —S^{a′″}—, —P^{a′}, -P^{a″}, -P^{a′″} and a are used as defined for formula (a).

Optionally, the moiety of formula (f) is substituted with one or more substituents.

In certain embodiments, BP^{a}, —S^{a}—, —S^{a′}—, —S^{a″}—, —S^{a′″}—, -P^{a′}, -P^{a″} and -P^{a′″} of formula (f) are as defined above for formula (a).

In certain embodiments, BP^{f} of formula (f) is —CR< and r is 0. In certain embodiments, —R is —H.

In certain embodiments, —S^{f}— of formula (f) is a chemical bond.

In certain embodiments, —Z^{a′}, —Z^{a″} and —Z^{a′″} of formula (f) have the same structure. In certain embodiments, —Z^{a′}, —Z^{a″} and —Z^{a′″} of formula (f) are of formula (b).

In certain embodiments, b1, b2, b3 and b4 are as described for formula (b).

In certain embodiments, —S^{f}— of formula (f) is a chemical bond, BP^{a} of formula (f) is —CR< with —R being —H. In certain embodiments, —S^{f}— of formula (f) is a chemical bond, BP^{a} of formula (f) is —CR< with —R being —H and —Z^{a′}, —Z^{a″} and —Z^{a′″} of formula (f) are of formula (b). In certain embodiments, —Z is of formula (g):

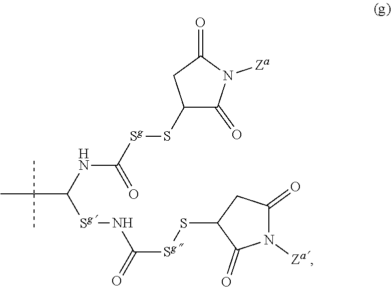

wherein
the dashed line indicates attachment to -L²-;
—S^{g}—, —S^{g′}— and —S^{g″}— are independently selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
   each R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)S(O)₂R^{3a}, —N(R³)S(O)R^{3a}, —N(R³)C(O) OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different;
and —Z^{a} and —Z^{a′} are independently

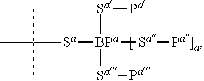

wherein
BP^{a}, —S^{a}—, —S^{a′}—, —S^{a″}—, —S^{a′″}—, —P^{a′}, -P^{a″}, -P^{a′″} and a are used as defined for formula (a)

Optionally, the moiety of formula (g) is substituted with one or more substituents.

In certain embodiments, BP^{a}, —S^{a}—, —S^{a′}—, —S^{a″}—, —S^{a′″}—, —P^{a′}, —P^{a″} and -P^{a′″} of formula (g) are as defined above for formula (a).

In certain embodiments, —S^{g}— of formula (g) is selected from the group consisting of C₁₋₆ alkyl, C₂₋₆ alkenyl and C₂₋₆ alkynyl, which are optionally substituted with one or more —R¹, which is the same or different,
wherein —R¹ is selected from the group consisting of halogen, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O) OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and —R³, —R^{3a} and —R^{3b} are independently selected from —H, methyl, ethyl, propyl and butyl.

In certain embodiments, —S^{g}— of formula (g) is selected from C₁₋₆ alkyl.

In certain embodiments, —S^{g′}— of formula (g) is selected from the group consisting of C₁₋₆ alkyl, C₂₋₆ alkenyl and C₂₋₆ alkynyl, which are optionally substituted with one or more —R¹, which is the same or different,
wherein —R¹ is selected from the group consisting of halogen, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O) OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and —R³, —R^{3a} and —R^{3b} are independently selected from —H, methyl, ethyl, propyl and butyl.

In certain embodiments, —S^{g′}— of formula (g) is selected from C₁₋₆ alkyl.

In certain embodiments, —S^{g″}— of formula (g) is selected from the group consisting of C₁₋₆ alkyl, C₂₋₆ alkenyl and C₂₋₆ alkynyl, which are optionally substituted with one or more —R¹, which is the same or different, wherein-R¹ is selected from the group consisting of halogen, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O) OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and —R³, —R^{3a} and —R^{3b} are independently selected from —H, methyl, ethyl, propyl and butyl.

In certain embodiments, —S^{g″}— of formula (g) is selected from C₁₋₆ alkyl.

In certain embodiments, —Z^{a} and —Z^{a′} of formula (g) have the same structure. In certain embodiments, —Z^{a} and —Z^{a′} of formula (g) are of formula (b).

In certain embodiments, —Z of formula (Ia) or (Ib) is of formula (g-i):

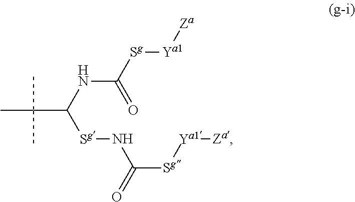

wherein
the dashed line indicates attachment to -L²-;
—S^{g}—, —S^{g′}— and —S^{g″}— are independently selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different; each R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O) OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different;
Y^{a1}— and —Y^{a1′}— are

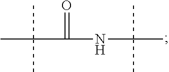

and —Z^{a} and —Z^{a} are independently

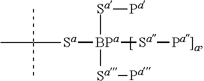

wherein
BP^{a}, —S^{a}—, —S^{a′}—, —S^{a″}—, —S^{a′″}—, —P^{a′}, -P^{a″}, -P^{a′″} and a are used as defined for formula (a).

Optionally, the moiety of formula (g-i) is substituted with one or more substituents.

In certain embodiments, —Y^{a1}— and —Y^{a1′}— of formula (g-i) are both

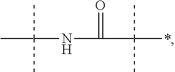

wherein the dashed line marked with the asterisk indicates the attachment to —Z^{a} or —Z^{a′}, respectively.

In certain embodiments, BP^{a}, —S^{a}—, —S^{a′}—, —S^{a″}—, —S^{a′″}—, —P^{a′}, -P^{a″} and -P^{a′″} of formula (g-i) are as defined above for formula (a).

In certain embodiments, —S^{g}—, —S^{g′}— and —S^{g″}— of formula (g-i) are as defined for formula (g). In certain embodiments, —Z^{a} and —Z^{a′} of formula (g-i) have the same structure. In certain embodiments, —Z^{a} and —Z^{a′} of formula (g-i) are of formula (b). In certain embodiments, for b1, b2, b3 and b4 are as described for formula (b).

In certain embodiments, —Z is of formula (h):

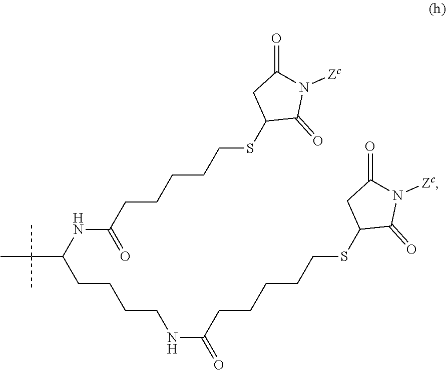

wherein
the dashed line indicates attachment to -L²-; and
each —Z^{c} is a moiety

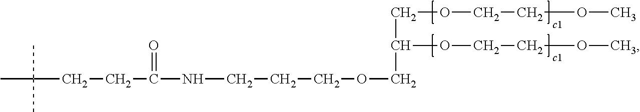

wherein
each c1 is an integer independently ranging from about 200 to 250.

Optionally, the moiety of formula (h) is substituted with one or more substituents.

In certain embodiments, both c1 of formula (h) are the same. In certain embodiments, both c1 of formula (h) are about 225.

In certain embodiments, —Z of formula (Ia) or (Ib) is of formula (h-a):

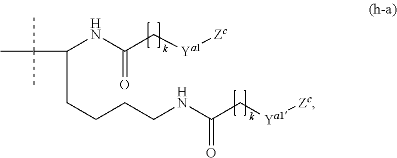

wherein
the dashed line indicates attachment to -L²-;
each k is independently of each other selected from the group consisting of 1, 2, 3, 4, 5, 6, 7, 8, 9, 10, 11 and 12;
—Y^{a1}— and —Y^{a1′}— are

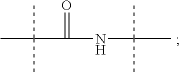

and
each —Z^{c} is a moiety

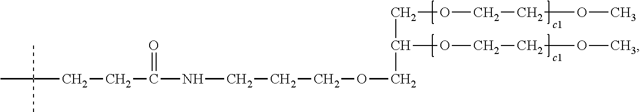

wherein
each c1 is an integer independently ranging from about 200 to 250.

Optionally, the moiety of formula (h-a) is substituted with one or more substituents.

In certain embodiments, each k of formula (h-a) is independently selected from the group consisting of 2, 3, 4, 5, 6 and 7. In certain embodiments, both k of formula (h-a) are identical.

In certain embodiments, both c1 of formula (h-a) are the same.

In certain embodiments, both c1 of formula (h-a) are about 225.

In certain embodiments, —Y^{a1}— and —Y^{a1′}— of formula (h-a) are both

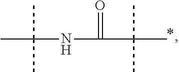

wherein the dashed line marked with the asterisk indicates the attachment to —Z^{c}.

In certain embodiments, the moiety —Z is of formula (h-i):

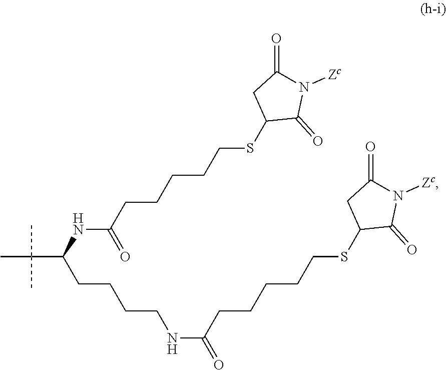

wherein
the dashed line indicates attachment to -L²-; and
each —Z^{c} is a moiety

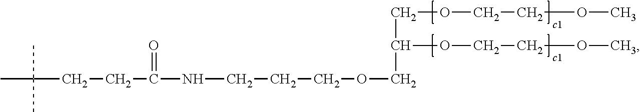

wherein each c1 is an integer independently ranging from 200 to 250.

Optionally, the moiety of formula (h-i) is substituted with one or more substituents.

In certain embodiments, both c1 of formula (h-i) are the same. In certain embodiments, both cl of formula (h-i) are about 225.

In certain embodiments, the moiety —Z of formula (Ia) or (Ib) is of formula (h-ia):

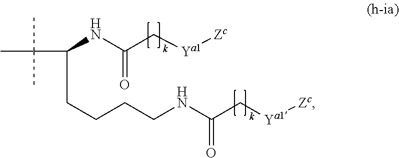

wherein
the dashed line indicates attachment to -L²-;
each k is independently of each other selected from the group consisting of 1, 2, 3, 4, 5, 6, 7, 8, 9, 10, 11 and 12;
—Y^{a1}— and —Y^{a1′}— are

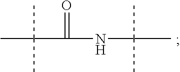

and
each —Z^{c} is a moiety

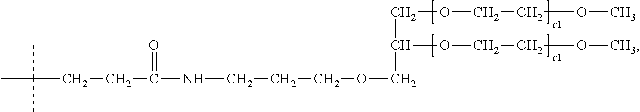

wherein each c1 is an integer independently ranging from 200 to 250.

In certain embodiments, each k of formula (h-ia) is independently selected from the group consisting of 2, 3, 4, 5, 6 and 7. In certain embodiments, both k of formula (h-ia) are identical.

In certain embodiments, both c1 of formula (h-ia) are the same. In certain embodiments, both c1 of formula (h-ia) are about 225.

In certain embodiments, —Y^{a1}— and —Y^{a1′}— of formula (h-ia) are both

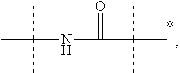

wherein the dashed line marked with the asterisk indicates the attachment to —Z^{c}.

In certain embodiments, —Z of formula (Ia) or (Ib) comprises a moiety selected from the group consisting of:

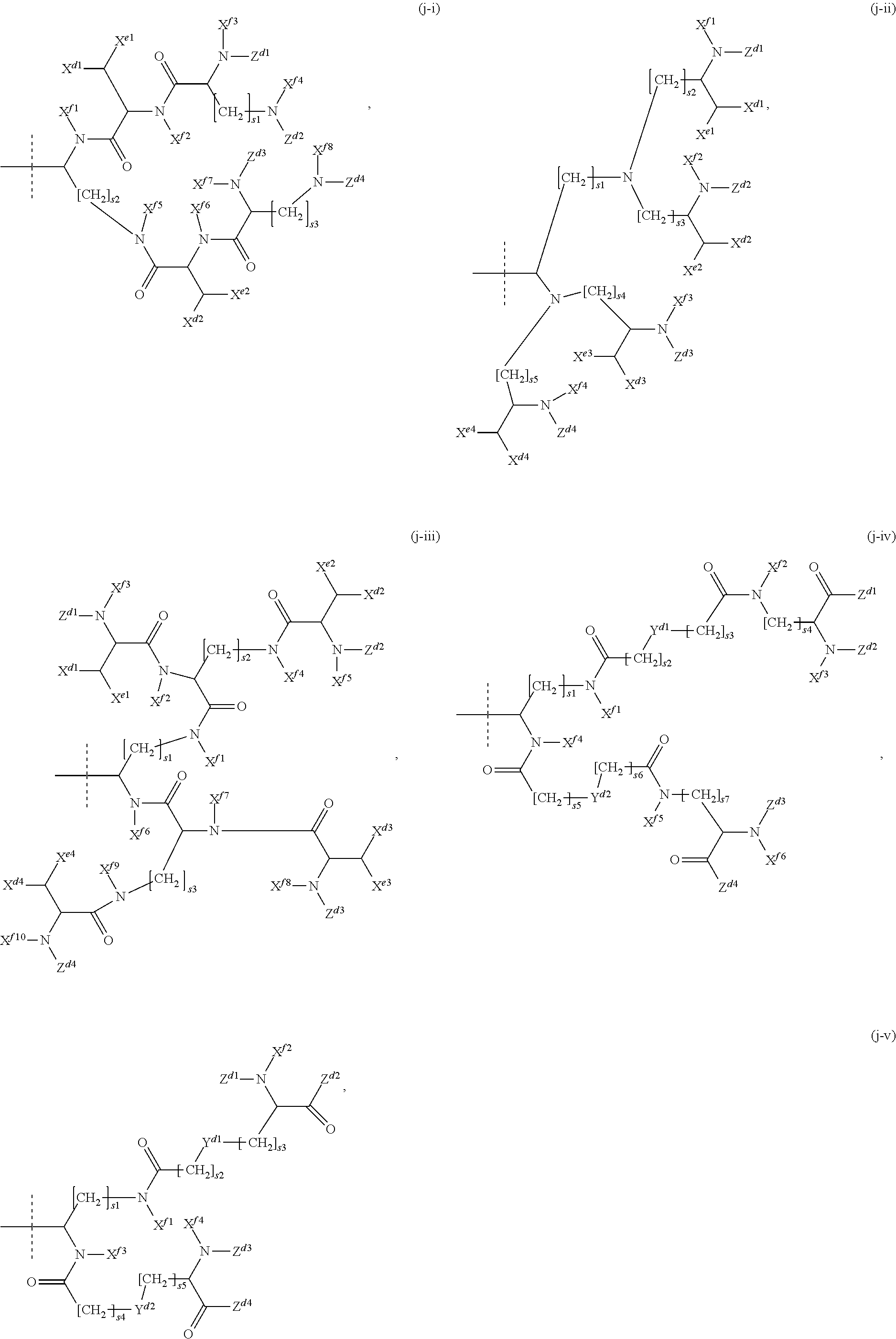

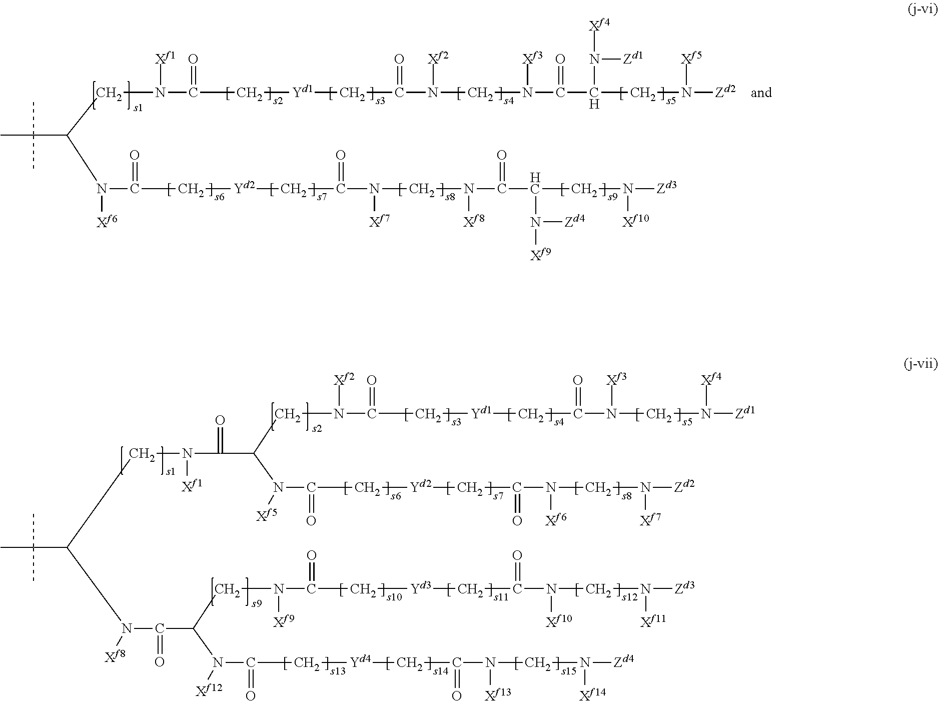

wherein
the dashed line indicates attachment to -L²-;
s1, s2, s3, s4, s5, s6, s7, s8, s9, s10, s11, s12, s13, s14 and s15 are independently of each other selected from the group consisting of 1, 2, 3, 4, 5, 6, 7, 8, 9 and 10;
—X^{d1}, —X^{d2}, —X^{d3} and —X^{d4} are independently of each other selected from the group consisting of —OH, —SH and —NR^{g1}R^{g2}; preferably —OH;
—X^{e1}, —X^{e2}, —X^{e3} and —X^{e4} are independently of each other selected from the group consisting of —H, C₁₋₆ alkyl, C₂₋₆ alkenyl and C₂₋₆ alkynyl;
—R^{g1} and —R^{g2} are independently of each other selected from the group consisting of —H, C₁₋₆ alkyl, C₂₋₆ alkenyl and C₂₋₆ alkynyl;
—X^{f1}, —X^{f2}, —X^{f3}, —X^{f4}, —X^{f5}, —X^{f6}, —X^{f7}, —X^{f8}, —X^{f9}, —X^{f10}, —X^{f11}, —X^{f12}, —X^{f13} and —X^{f14} are independently of each other selected from the group consisting of —H, C₁₋₆ alkyl, C₂₋₆ alkenyl and C₂₋₆ alkynyl; preferably —H;
—Y^{d1}—, —Y^{d2}—, —Y^{d3}— and —Y^{d4}— are independently of each other selected from the group consisting of

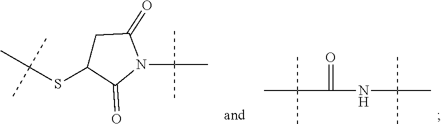

and
—Z^{d1}, —Z^{d2}, —Z^{d3} and —Z^{d4} are independently of each other a protein, in certain embodiments a random coil protein and in certain embodiments, a random coil protein selected from the group consisting of PA, PAS, PAG, PG and XTEN™.

In certain embodiments, —Y^{d1}— and —Y^{d2}— of formula (j-iv), (j-v) and (j-vi) and —Y^{d1}—, —Y^{d2}—, —Y^{d3}— and —Y^{d4}— of formula (j-vii) are

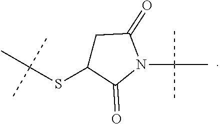

In certain embodiments, —Y^{d1}— and —Y^{d2}— of formula (j-iv), (j-v) and (j-vi) and —Y^{d1}—, —Y^{d2}—, —Y^{d3}— and —Y^{d4}— of formula (j-vii) are

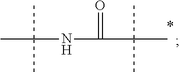

wherein the dashed line marked with the asterisk is oriented towards —Z^{d1}, —Z^{d2}, —Z^{d3} and —Z^{d4}, respectively, and the unmarked dashed line is oriented towards -L²-.

In certain embodiments, —X^{f1}, —X^{f2}, —X^{f3}, —X^{f4}, —X^{f5}, —X^{f6}, —X^{f7} and —X^{f8} of formula (j-i) are —H; —X^{d1} and —X^{d2} of formula (j-i) are —OH; —X^{e1} and —X^{e2} of formula (j-i) are selected from the group consisting of —H and methyl; and s1, s2 and s3 of formula (j-i) are selected from the group consisting of 2, 3, 4, 5 and 6.

In certain embodiments, —X^{f1}, —X^{f2}, —X^{f3}, —X^{f4}, —X^{f5}, —X^{f6}, —X^{f7} and —X^{f8} of formula (j-i) are —H; —X^{d1} and —X^{d2} of formula (j-i) are —OH; —X^{e1} and —X^{e2} of formula (j-i) are —H; and s1, s2 and s3 of formula (j-i) are 4.

In certain embodiments, —X^{f1}, —X^{f2}, —X^{f3} and —X^{f4} of formula (j-ii) are —H; —X^{d1}, —X⁴², —X^{d3} and —X^{d4} of formula (j-ii) are —OH; —X^{e1}, —X^{e2}, —X^{e3} and —X^{e4} of formula (j-ii) are selected from the group consisting of —H and methyl; s1, s2, s3, s4 and s5 of formula (j-ii) are selected from the group consisting of 1, 2, 3, 4, 5 and 6. In certain embodiments, —X^{f1}, —X^{f2}, —X^{f3} and —X^{f4} of formula (j-ii) are —H; —X^{d1}, —X^{d2}, —X^{d3} and —X⁴⁴ of formula (j-ii) are —OH; —X^{e1}, —X^{e2}, —X^{e3} and —X^{e4} of formula (j-ii) are —H; s1 is 4 of formula (j-ii) and s2, s3, s4 and s5 of formula (j-ii) are 1.

In certain embodiments, —X^{f1}, —X^{f2}, —X^{f3}, —X^{f4}, —X^{f5}, —X^{f6}, —X^{f7}, —X^{f8}, —X^{f9} and —X^{f10} of formula (j-iii) are —H; —X^{d1}, —X^{d2}, —X^{d3} and —X⁴⁴ of formula (j-iii) are —OH; —X^{e1}, —X^{e2}, —X^{e3} and —X^{e4} of formula (j-iii) are selected from the group consisting of —H and methyl; and s1, s2 and s3 of formula (j-iii) are selected from the group consisting of 2, 3, 4, 5 and 6. In certain embodiments, —X^{f1}, —X^{f2}, —X^{f3}, —X^{f4}, —X^{f5}, —X^{f6}, —X^{f7}, —X^{f8}, —X^{f9} and —X^{f10} of formula (j-iii) are —H; —X^{d1}, —X⁴², —X^{d3} and —X^{d4} of formula (j-iii) are —OH; —X^{e1}, —X^{e2}, —X^{e3} and —X^{e4} of formula (j-iii) are —H; and s1, s2 and s3 of formula (j-iii) are 4.

In certain embodiments, —X^{f1}, —X^{f2}, —X^{f3}, —X^{f4}, —X^{f5} and —X^{f6} of formula (j-iv) are —H; s1, s2, s3, s4, s5, s6 and s7 of formula (j-iv) are selected from the group consisting of 1, 2, 3, 4, 5, 6 and 7; —Y^{d1}— and —Y^{d2}— are selected from the group consisting of

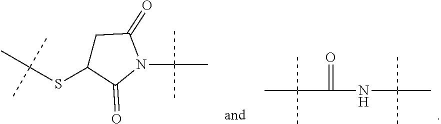

In certain embodiments, —X^{f1}, —X^{f2}, —X^{f3}, —X^{f4}, —X^{f5} and —X^{f6} of formula (j-iv) are —H; s1 of formula (j-iv) is 3, s2 of formula (j-iv) is 5, s3 of formula (j-iv) is 2, s4 of formula (j-iv) is 4, s5 of formula (j-iv) is 5, s6 of formula (j-iv) is 2 and s7 of formula (j-iv) is 4; and —Y^{d1}— and —Y^{d2}— of formula (j-iv) are

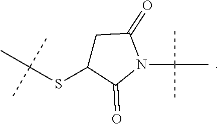

In certain embodiments, —X^{f1}, —X^{f2}, —X^{f3}, —X^{f4}, —X^{f5} and —X^{f6} of formula (j-iv) are —H; s1 of formula (j-iv) is 3, s2 of formula (j-iv) is 5, s3 of formula (j-iv) is 2, s4 of formula (j-iv) is 4, s5 of formula (j-iv) is 5, s6 of formula (j-iv) is 2 and s7 of formula (j-iv) is 4; and —Y^{d1}— and —Y^{d2}— of formula (j-iv) are

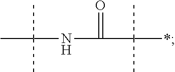

wherein the dashed line marked with the asterisk is oriented towards-Z^{d1}, —Z^{d2}, —Z^{d3} and —Z^{d4}, respectively, and the unmarked dashed line is oriented towards -L²-.

In certain embodiments, —X^{f1}, —X^{f2}, —X^{f3} and —X^{f4} of formula (j-v) are —H; s1, s2, s3, s4 and s5 of formula (j-v) are selected from the group consisting of 1, 2, 3, 4, 5, 6 and 7; —Y^{d1}— and —Y^{d2}— of formula (j-v) are selected from the group consisting of

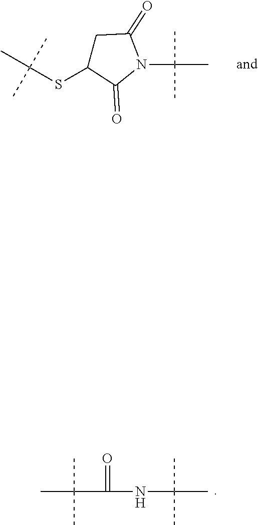

In certain embodiments, —X^{f1}, —X^{f2}, —X^{f3} and —X^{f4} of formula (j-v) are —H; s1 of formula (j-v) is 3, s2 of formula (j-v) is 2, s3 of formula (j-v) is 1, s4 of formula (j-v) is 2 and s5 of formula (j-v) is 1; and —Y^{d1}- and —Y^{d2}— of formula (j-v) are

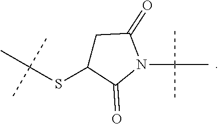

In certain embodiments, —X^{f1}, —X^{f2}, —X^{f3} and —X^{f4} of formula (j-v) are —H; s1 of formula (j-v) is 3, s2 of formula (j-v) is 2, s3 of formula (j-v) is 1, s4 of formula (j-v) is 2 and s5 of formula (j-v) is 1; and —Y^{d1}— and —Y^{d2}— of formula (j-v) are

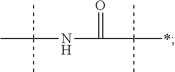

wherein the dashed line marked with the asterisk is oriented towards —Z^{d1}, —Z^{d2}, —Z^{d3} and —Z^{d4}, respectively, and the unmarked dashed line is oriented towards -L²-.

In certain embodiments, —X^{f1}, —X^{f2}, —X^{f3}, —X^{f4}, —X^{f5}, —X^{f6}, —X^{f7}, —X^{f8}, —XP and —X^{f10} of formula (j-vi) are —H; s1, s2, s3, s4, s5, s6, s7, s8 and s9 of formula (j-vi) are selected from the group consisting of 1, 2, 3, 4, 5, 6 and 7; —Y^{d1}— and —Y^{d2}— of formula (j-vi) are selected from the group consisting of

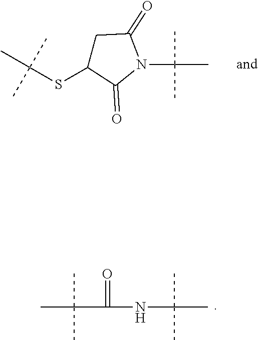

In certain embodiments, —X^{f1}, —X^{f2}, —X^{f3}, —X^{f4}, —X^{f5}, —X^{f6}, —X^{f7}, —X^{f8}, —X^{f9} and —X^{f10} of formula (j-vi) are —H; s1 of formula (j-vi) is 4, s2 of formula (j-vi) is 5, s3 of formula (j-vi) is 2, s4 of formula (j-vi) is 4, s5 of formula (j-vi) is 4, s6 of formula (j-vi) is 5, s7 of formula (j-vi) is 2, s8 of formula (j-vi) is 4 and s9 of formula (j-vi) is 4; and —Y^{d1}— and —Y^{d2}— of formula (j-v) are

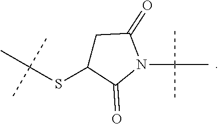

In certain embodiments, —X^{f1}, —X^{f2}, —X^{f3}, —X^{f4}, —X^{f5}, —X^{f6}, —X^{f7}, —X^{f8}, —X^{f9} and —X^{f10} of formula (j-vi) are —H; s1 of formula (j-vi) is 4, s2 of formula (j-vi) is 5, s3 of formula (j-vi) is 2, s4 of formula (j-vi) is 4, s5 of formula (j-vi) is 4, s6 of formula (j-vi) is 5, s7 of formula (j-vi) is 2, s8 of formula (j-vi) is 4 and s9 of formula (j-vi) is 4; and —Y^{d1}— and —Y^{d2}— of formula (j-v) are

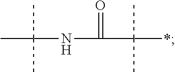

wherein the dashed line marked with the asterisk is oriented towards —Z^{d1}, —Z^{d2}, —Z^{d3} and —Z^{d4}, respectively, and the unmarked dashed line is oriented towards -L²-.

In certain embodiments, —X^{f1}, —X^{f2}, —X^{f3}, —X^{f4}, —X^{f5}, —X^{f6}, —X^{f7}, —X^{f8}, —X^{f9}, —X^{f10}, —X^{f11}, —X^{f12}, —X^{f13} and —X^{f14} of formula (j-vii) are —H; s1, s2, s3, s4, s5, s6, s7, s8, s9, s10, s11, s12, s13, s14 and s15 of formula (j-vii) are selected from the group consisting of 1, 2, 3, 4, 5, 6 and 7; —Y^{d1}—, —Y^{d2}—, —Y^{d3}— and —Y^{d4}— of formula (j-vii) are selected from the group consisting of

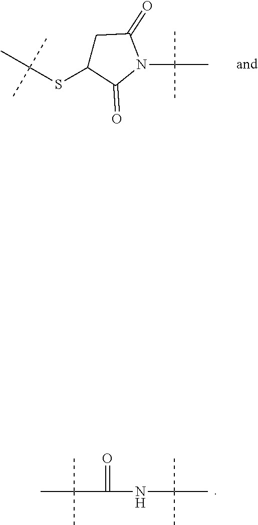

In certain embodiments, —X^{f1}, —XP2, —X^{f3}, —X^{f4}, —X^{f5}, —X^{f6}, —X^{f7}, —X^{f8}, —X^{f9}, —X^{f10}, —X^{f11}, —X^{f12}, —X^{f13} and —X^{f14} of formula (j-vii) are —H; are —H; s1 of formula (j-vii) is 4, s2 of formula (j-vii) is 4, s3 of formula (j-vii) is 5, s4 of formula (j-vii) is 2, s5 of formula (j-vii) is 4, s6 of formula (j-vii) is 5, s7 of formula (j-vii) is 2, s8 of formula (j-vii) is 4, s9 of formula (j-vii) is 4, s10 of formula (j-vii) is 5, s11 of formula (j-vii) is 2, s12 of formula (j-vii) is 4, s13 of formula (j-vii) is 5, s14 of formula (j-vii) is 2 and s15 of formula (j-vii) is 4; and —Y^{d1}—, —Y^{d2}—, —Y^{d3}— and —Y^{d4}— of formula (j-vii) are

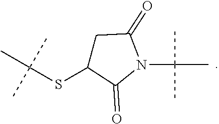

In certain embodiments, —X^{f1}, —X^{f2}, —X^{f3}, —X^{f4}, —X^{f5}, —X^{f6}, —X^{f7}, —X^{f8}, —XP, —X^{f10}, —X^{f11}, —X^{f12}, —X^{f13} and —X^{f14} of formula (j-vii) are —H; are —H; s1 of formula (j-vii) is 4, s2 of formula (j-vii) is 4, s3 of formula (j-vii) is 5, s4 of formula (j-vii) is 2, s5 of formula (j-vii) is 4, s6 of formula (j-vii) is 5, s7 of formula (j-vii) is 2, s8 of formula (j-vii) is 4, s9 of formula (j-vii) is 4, s10 of formula (j-vii) is 5, s11 of formula (j-vii) is 2, s12 of formula (j-vii) is 4, s13 of formula (j-vii) is 5, s14 of formula (j-vii) is 2 and s15 of formula and —Y^{d1}—, —Y^{d2}—, —Y^{d3}— and —Y^{d4}— of formula (j-vii) are

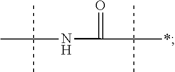

wherein the dashed line marked with the asterisk is oriented towards —Z^{d1}, —Z^{d2}, —Z^{d3} and —Z^{d4} respectively, and the unmarked dashed line is oriented towards -L²-.

In certain embodiments, —Z^{d1}, —Z^{d2}, —Z^{d3} and —Z^{d4} of formula (j-i), (j-ii), (j-iii), (j-iv), (j-v), (j-vi) and (j-vii) have the same structure. In certain embodiments, —Z^{d1}, —Z^{d2}, —Z^{d3} and —Z^{d4} of formula (j-i), (j-ii), (j-iii), (j-iv), (j-v), (j-vi) and (j-vii) are a PA moiety. In certain embodiments, —Z^{d1}, —Z^{d2}, —Z^{d3} and —Z^{d4} of formula (j-i), (j-ii), (j-iii), (j-iv), (j-v), (j-vi) and (j-vii) are a PAS moiety. In certain embodiments, —Z^{d1}, —Z^{d2}, —Z^{d3} and —Z^{d4} of formula (j-i), (j-ii), (j-iii), (j-iv), (j-v), (j-vi) and (j-vii) are a PAG moiety. In certain embodiments, —Z^{d1}, —Z^{d2}, —Z^{d3} and —Z^{d4} of formula (j-i), (j-ii), (j-iii), (j-iv), (j-v), (j-vi) and (j-vii) are a PG moiety. In certain embodiments, —Z^{d1}, —Z^{d2}, —Z^{d3} and —Z^{d4} of formula (j-i), (j-ii), (j-iii), (j-iv), (j-v), (j-vi) and (j-vii) are an XTEN™ moiety.

In certain embodiments, the reversible CNP conjugate is of formula (Iif):

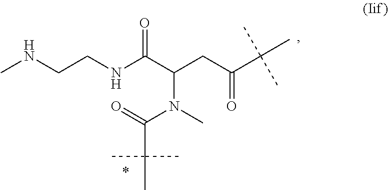

wherein
the unmarked dashed line indicates the attachment to a nitrogen of -D which is a CNP moiety by forming an amide bond; and
the dashed line marked with the asterisk indicates attachment to —Z having the structure

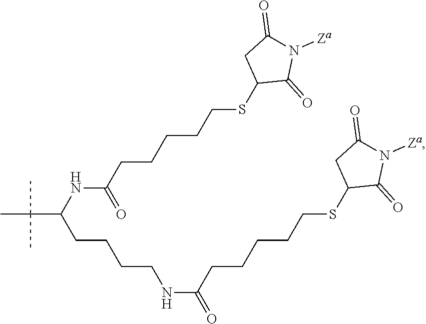

wherein
each —Z^{a} is

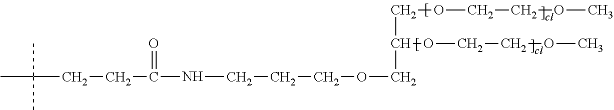

wherein
each c1 is an integer independently ranging from 200 to 250.

In certain embodiments, each c1 of formula (Iif) is about 225.

In certain embodiments, the reversible CNP conjugate is of formula (Iif-i):

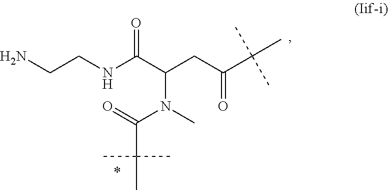

wherein
the unmarked dashed line indicates the attachment to a nitrogen of -D which is a CNP moiety by forming an amide bond; and
the dashed line marked with the asterisk indicates attachment to —Z having the structure

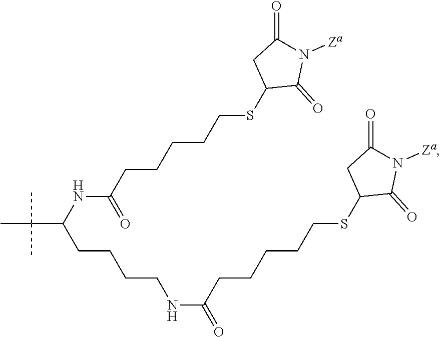

wherein
each —Z^{a} is

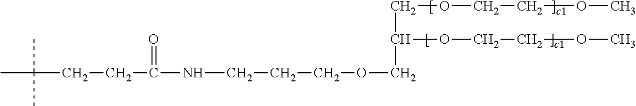

wherein
each c1 is an integer independently ranging from 200 to 250.

In certain embodiments, each c1 of formula (Iif-i) is about 225.

In certain embodiments, the CNP conjuagate is of formula (Iif-ii):

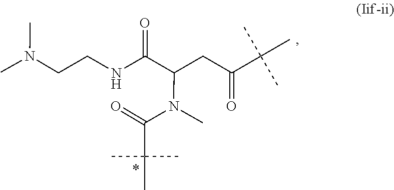

wherein
the unmarked dashed line indicates the attachment to a nitrogen of -D which is a CNP moiety by forming an amide bond; and
the dashed line marked with the asterisk indicates attachment to —Z having the structure

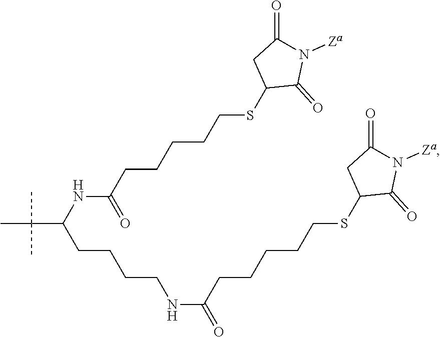

wherein
each —Z^{a} is

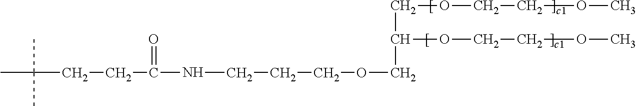

wherein
each c1 is an integer independently ranging from 200 to 250.

In certain embodiments, each c1 of formula (Iif-ii) is about 225.

In certain embodiments, -D of formula (Iif), (Iif-i) and (Iif-ii) is a CNP moiety, i.e. the reversible conjugate of formula (Iif), (Iif-i) and (Iif-ii) is a CNP conjugate. In certain embodiments, -D of formula (Iif), (Iif-i) and (Iif-ii) is a CNP moiety having the sequence of SEQ ID NO:24, SEQ ID NO:25 or SEQ ID NO:30. In certain embodiments, -D of formula (Iif), (Iif-i) and (Iif-ii) is a CNP moiety having the sequence of SEQ ID NO:24. In certain embodiments, -D of formula (Iif), (Iif-i) and (Iif-ii) is a CNP moiety having the sequence of SEQ ID NO:20. In certain embodiments, -D of formula (Iif), (Iif-i) and (Iif-ii) is a CNP moiety having the sequence of SEQ ID NO:21. In certain embodiments, -D of formula (Iif), (Iif-i) and (Iif-ii) is a CNP moiety having the sequence of SEQ ID NO:22. -D of formula (Iif), (Iif-i) and (Iif-ii) is a CNP moiety having the sequence of SEQ ID NO:23. In certain embodiments, -D of formula (Iif), (Iif-i) and (Iif-ii) is a CNP moiety having the sequence of SEQ ID NO:30.

In certain embodiments, -D of formula (Iif), (Iif-i) and (Iif-ii) is a CNP moiety which is attached to -L¹-through the nitrogen of the N-terminal amine functional group of CNP.

In certain embodiments, -D of formula (Iif), (Iif-i) and (Iif-ii) is a CNP moiety which is attached to -L¹-through a nitrogen provided by the amine functional group of a lysine side chain of the CNP moiety.

In certain embodiments, said lysine side chain is not part of the ring formed by the disulfide bridge between the cysteine residues at positions 22 and 38, if the CNP moiety is of SEQ ID NO: 24.

Accordingly, in certain embodiments, the CNP moiety is connected to -L¹- in the reversible CNP conjugate of formula (Iif), (Iif-i) and (Iif-ii) through the amine functional group provided by the side chain of the lysine at position 9, if the CNP has the sequence of SEQ ID NO: 24.

In certain embodiments, the CNP moiety is connected to -L¹- in the reversible CNP conjugate of formula (Iif), (Iif-i) and (Iif-ii) through the amine functional group provided by the side chain of the lysine at position 11, if the CNP has the sequence of SEQ ID NO:24.

In certain embodiments, the CNP moiety is connected to -L¹- in the reversible CNP conjugate of formula (Iif), (Iif-i) and (Iif-ii) through the amine functional group provided by the side chain of the lysine at position 15, if the CNP has the sequence of SEQ ID NO:24.

In certain embodiments, the CNP moiety is connected to -L¹- in the reversible CNP conjugate of formula (Iif), (Iif-i) and (Iif-ii) through the amine functional group provided by the side chain of the lysine at position 16, if the CNP has the sequence of SEQ ID NO:24.

In certain embodiments, the CNP moiety is connected to -L¹- in the reversible CNP conjugate of formula (Iif), (Iif-i) and (Iif-ii) through the amine functional group provided by the side chain of the lysine at position 20, if the CNP has the sequence of SEQ ID NO:24.

In certain embodiments, said lysine side chain is part of the ring formed by the disulfide bridge between the cysteine residues at positions 22 and 38, if the CNP moiety is of SEQ ID NO: 24.

Accordingly, in certain embodiments, the CNP moiety is connected to -L¹- in the CNP conjugate of formula (Iif) through the amine functional group provided by the side chain of the lysine at position 26, if the CNP has the sequence of SEQ ID NO:24.

In certain embodiments, the reversible CNP conjugate is of formula (Iif-i), wherein c1 is about 225, the CNP moiety has the sequence of SEQ ID NO:24 and is attached to -L¹-through the amine functional group provided by the side chain of the lysine at position 26.

In certain embodiments, the reversible CNP conjugate is of formula (Iif), wherein c1 is about 225, -D is a CNP moiety having the sequence of SEQ ID NO:20 and is attached to -L¹-through the amine functional group provided by the side chain of the lysine at position 30.

In certain embodiments, the reversible CNP conjugate is of formula (Iif-i), wherein c1 is about 225, -D is a CNP moiety having the sequence of SEQ ID NO:20 and is attached to -L¹-through the amine functional group provided by the side chain of the lysine at position 30.

In certain embodiments, the reversible CNP conjugate is of formula (Iif-ii), wherein c1 is about 225, the CNP moiety has the sequence of SEQ ID NO:20 and is attached to -L¹-through the amine functional group provided by the side chain of the lysine at position 30.

In certain embodiments, the reversible CNP conjugate is of formula (Iif), wherein c1 is about 225, -D is a CNP moiety having the sequence of SEQ ID NO:21 and is attached to -L¹-through the amine functional group provided by the side chain of the lysine at position 29.

In certain embodiments, the reversible CNP conjugate is of formula (Iif-i), wherein c1 is about 225, the CNP moiety has the sequence of SEQ ID NO:21 and is attached to -L¹-through the amine functional group provided by the side chain of the lysine at position 29.

In certain embodiments, the reversible CNP conjugate is of formula (Iif-ii), wherein c1 is about 225, the CNP moiety has the sequence of SEQ ID NO:21 and is attached to -L¹-through the amine functional group provided by the side chain of the lysine at position 29.

In certain embodiments, the reversible CNP conjugate is of formula (Iif), wherein c1 is about 225, -D is a CNP moiety having the sequence of SEQ ID NO:22 and is attached to -L¹-through the amine functional group provided by the side chain of the lysine at position 28.

In certain embodiments, the reversible CNP conjugate is of formula (Iif-i), wherein c1 is about 225, the CNP moiety has the sequence of SEQ ID NO:22 and is attached to -L¹-through the amine functional group provided by the side chain of the lysine at position 28.

In certain embodiments, the reversible CNP conjugate is of formula (Iif-ii), wherein c1 is about 225, the CNP moiety has the sequence of SEQ ID NO:22 and is attached to -L¹-through the amine functional group provided by the side chain of the lysine at position 28.

In certain embodiments, the reversible CNP conjugate is of formula (Iif), wherein c1 is about 225, -D is a CNP moiety having the sequence of SEQ ID NO:23 and is attached to -L¹-through the amine functional group provided by the side chain of the lysine at position 27.

In certain embodiments, the reversible CNP conjugate is of formula (Iif-i), wherein c1 is about 225, the CNP moiety has the sequence of SEQ ID NO:23 and is attached to -L¹-through the amine functional group provided by the side chain of the lysine at position 27.

In certain embodiments, the reversible CNP conjugate is of formula (Iif-ii), wherein c1 is about 225, the CNP moiety has the sequence of SEQ ID NO:23 and is attached to -L¹-through the amine functional group provided by the side chain of the lysine at position 27.

In certain embodiments, the reversible CNP conjugate is of formula (Iif), wherein c1 is about 225, -D is a CNP moiety having the sequence of SEQ ID NO:30 and is attached to -L¹-through the amine functional group provided by the side chain of the lysine at position 27.

In certain embodiments, the reversible CNP conjugate is of formula (Iif-i), wherein c1 is about 225, the CNP moiety has the sequence of SEQ ID NO:30 and is attached to -L¹-through the amine functional group provided by the side chain of the lysine at position 27.

In certain embodiments, the reversible CNP conjugate is of formula (Iif-ii), wherein c1 is about 225, the CNP moiety has the sequence of SEQ ID NO:30 and is attached to -L¹-through the amine functional group provided by the side chain of the lysine at position 27.

In certain embodiments, the reversible CNP conjugate is of formula (Iif-ii), wherein c1 is about 225, the CNP moiety has the sequence of SEQ ID NO:24 and is attached to -L¹-through the amine functional group provided by the side chain of the lysine at position 26.

It is understood that the positions of the cysteines and lysines mentioned above vary depending on the lengths of the CNP moiety and that the person skilled in the art will have no difficulty identifying the corresponding cysteines and lysines in longer or shorter versions of the CNP moiety and also understands that for example some lysines may not be present in shorter CNP moieties. It is further understood that as a result of for example site-directed mutagenesis there might be more lysine residues in the non-ring forming part and/or ring forming part of the CNP moiety.

In certain embodiments, the reversible CNP conjugate is of formula (Iif), wherein c1 is about 225, -D is a CNP moiety having the sequence of SEQ ID NO:24 and is attached to -L¹-through the amine functional group provided by the side chain of the lysine at position 26.

In certain embodiments, the reversible CNP conjugate is of formula (Iif′):

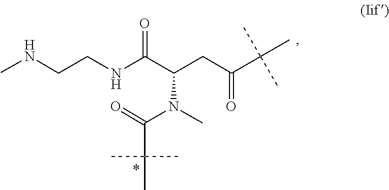

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

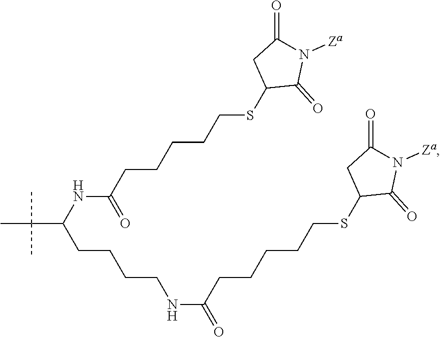

wherein
each —Z^{a} is

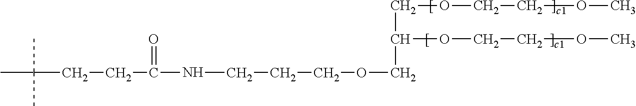

wherein
each c1 is an integer independently ranging from 200 to 250.

In certain embodiments, each c1 of formula (Iif′) is about 225.

In certain embodiments, the reversible CNP conjugate is of formula (Iif-i′):

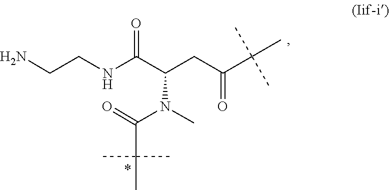

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

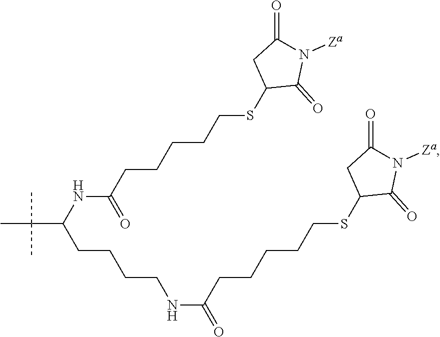

wherein
each —Z^{a} is

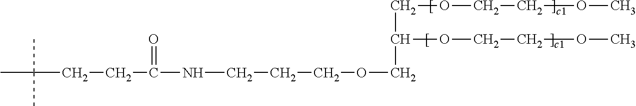

wherein
each c1 is an integer independently ranging from 200 to 250.

In certain embodiments, each c1 of formula (Iif-i′) is about 225.

In certain embodiments, the reversible CNP conjugate is of formula (Iif-ii′):

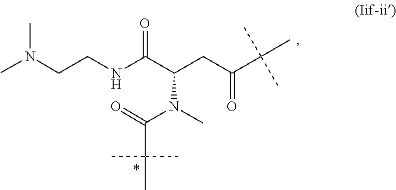

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

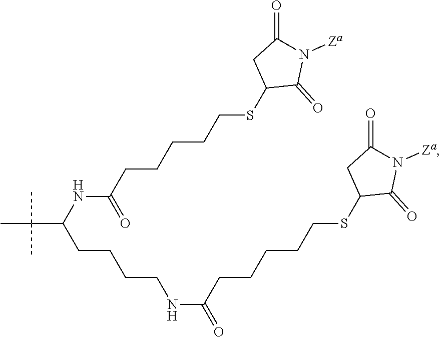

wherein
each —Z^{a} is

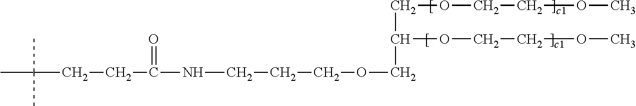

wherein
each c1 is an integer independently ranging from 200 to 250.

In certain embodiments, each c1 of formula (Iif-ii′) is about 225.

In certain embodiments, the reversible CNP conjugate is of formula (Iifa):

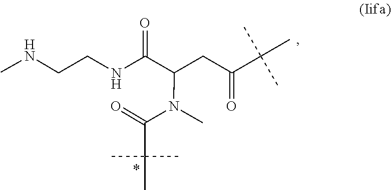

wherein
the unmarked dashed line indicates the attachment to a nitrogen of -D which is a CNP moiety by forming an amide bond; and
the dashed line marked with the asterisk indicates attachment to —Z having the structure

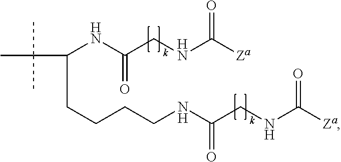

wherein
k is selected from the group consisting of 1, 2, 3, 4, 5, 6, 7, 8, 9, 10, 11 and 12;
each —Z^{a} is

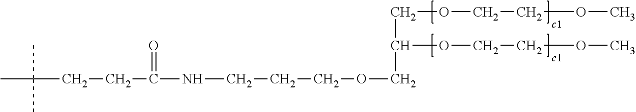

wherein
each c1 is an integer independently ranging from 200 to 250.

In certain embodiments, k of formula (Iifa) is selected from the group consisting of 2, 3, 4, 5, 6 and 7.

In certain embodiments, each c1 of formula (Iifa) is about 225.

In certain embodiments, the reversible CNP conjugate is of formula (Iifa-i):

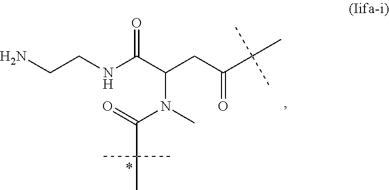

wherein
the unmarked dashed line indicates the attachment to a nitrogen of -D which is a CNP moiety by forming an amide bond; and
the dashed line marked with the asterisk indicates attachment to —Z having the structure

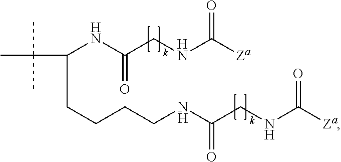

wherein
k is selected from the group consisting of 1, 2, 3, 4, 5, 6, 7, 8, 9, 10, 11 and 12;
each —Z^{a} is

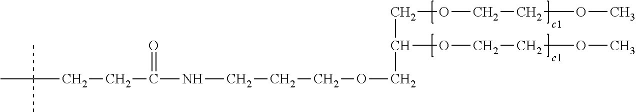

wherein
each c1 is an integer independently ranging from 200 to 250.

In certain embodiments, k of formula (Iifa-i) is selected from the group consisting of 2, 3, 4, 5, 6 and 7.

In certain embodiments, each c1 of formula (Iifa-i) is about 225.

In certain embodiments, the reversible CNP conjugate is of formula (Iifa-ii):

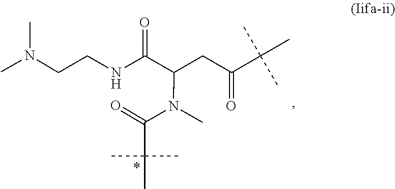

wherein
the unmarked dashed line indicates the attachment to a nitrogen of -D which is a CNP moiety by forming an amide bond; and
the dashed line marked with the asterisk indicates attachment to —Z having the structure

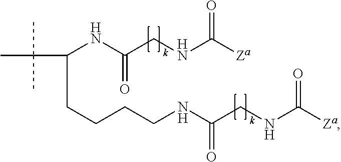

wherein
k is selected from the group consisting of 1, 2, 3, 4, 5, 6, 7, 8, 9, 10, 11 and 12;
each —Z^{a} is

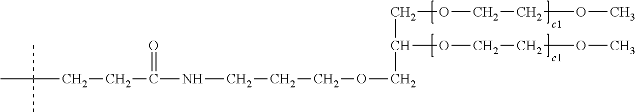

wherein
each c1 is an integer independently ranging from 200 to 250.

In certain embodiments, each c1 of formula (Iifa-ii) is about 225.

In certain embodiments, the CNP moiety of the reversible CNP conjugate of formula (Iifa), (Iifa-i) and (Iifa-ii) has the sequence of SEQ ID NO:25.

In certain embodiments, the CNP moiety of the reversible CNP conjugate of formula (Iifa), (Iifa-i) and (Iifa-ii) has the sequence of SEQ ID NO:20.

In certain embodiments, the CNP moiety of the reversible CNP conjugate of formula (Iifa), (Iifa-i) and (Iifa-ii) has the sequence of SEQ ID NO:21.

In certain embodiments, the CNP moiety of the reversible CNP conjugate of formula (Iifa), (Iifa-i) and (Iifa-ii) has the sequence of SEQ ID NO:22.

In certain embodiments, the CNP moiety of the reversible CNP conjugate of formula (Iifa), (Iifa-i) and (Iifa-ii) has the sequence of SEQ ID NO:23.

In certain embodiments, the CNP moiety of the reversible CNP conjugate of formula (Iifa), (Iifa-i) and (Iifa-ii) has the sequence of SEQ ID NO:30.

In certain embodiments, the CNP moiety of the reversible CNP conjugate of formula (Iifa), (Iifa-i) and (Iifa-ii) has the sequence of SEQ ID NO:24.

In certain embodiments, the CNP moiety is attached to -L¹- in the reversible CNP conjugate of formula (Iifa), (Iifa-i) and (Iifa-ii) through the nitrogen of the N-terminal amine functional group of CNP.

In certain embodiments, the reversible CNP conjugate is of formula (Iifa′):

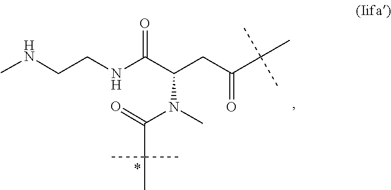

wherein
the unmarked dashed line indicates the attachment to the nitrogen provided by the side chain of the lysine at position 26 of the CNP moiety of SEQ ID NO:24 by forming an amide bond; and
the dashed line marked with the asterisk indicates attachment to —Z having the structure

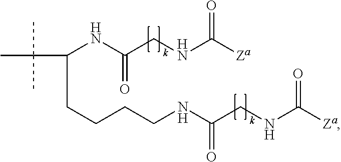

wherein
k is selected from the group consisting of 1, 2, 3, 4, 5, 6, 7, 8, 9, 10, 11 and 12;
each —Z^{a} is

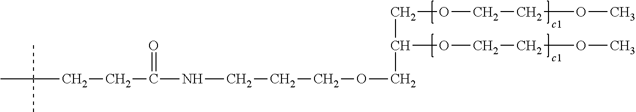

wherein
each c1 is an integer independently ranging from 200 to 250.

In certain embodiments, k of formula (Iifa′) is selected from the group consisting of 2, 3, 4, 5, 6 and 7.

In certain embodiments, each c1 of formula (Iifa′) is about 225.

In certain embodiments, the reversible CNP conjugate is of formula (Iifa-i′):

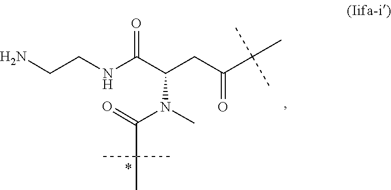

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

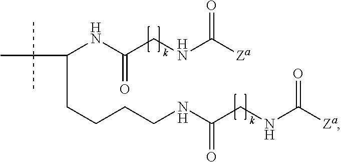

wherein
k is selected from the group consisting of 1, 2, 3, 4, 5, 6, 7, 8, 9, 10, 11 and 12;
each —Z^{a} is

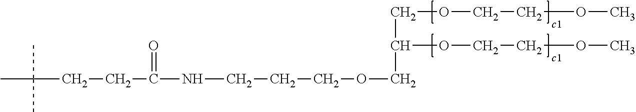

wherein
each c1 is an integer independently ranging from 200 to 250.

In certain embodiments, k of formula (Iifa-i′) is selected from the group consisting of 2, 3, 4, 5, 6 and 7.

In certain embodiments, each c1 of formula (Iifa-i′) is about 225.

In certain embodiments, the reversible CNP conjugate is of formula (Iifa-ii′):

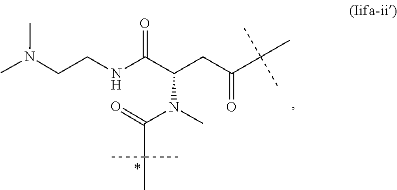

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

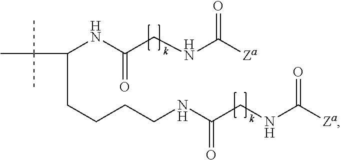

wherein
k is selected from the group consisting of 1, 2, 3, 4, 5, 6, 7, 8, 9, 10, 11 and 12;
each —Z^{a} is

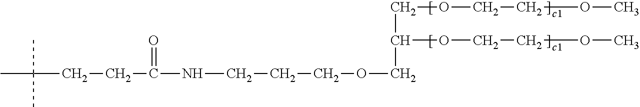

wherein
each c1 is an integer independently ranging from 200 to 250.

In certain embodiments, k of formula (Iifa-ii′) is selected from the group consisting of 2, 3, 4, 5, 6 and 7.

In certain embodiments, each c1 of formula (Iifa-ii′) is about 225.

In certain embodiments, the CNP compound is a CNP agonist selected from the group consisting of small molecules, natural products, oligonucleotides, polypeptides and proteins.

In certain embodiments, the CNP agonist comprises a small molecule. In certain embodiments, the CNP agonist is a small molecule.

In certain embodiments, the CNP agonist comprises a natural product. In certain embodiments, the CNP agonist is a natural product.

In certain embodiments, the CNP agonist comprises an oligonucleotide. In certain embodiments, such oligonucleotide is selected from the group consisting of antisense oligonucleotides, aptamers, RNAi and siRNA. In certain embodiments, the CNP agonist is an oligonucleotide selected from the group consisting of antisense oligonucleotides, aptamers, RNAi and siRNA.

In certain embodiments, the CNP agonist comprises a protein. In certain embodiments, the CNP agonist is a protein.

In certain embodiments, the CNP agonist comprises a polypeptide. In certain embodiments, the CNP agonist is a polypeptide.

In certain embodiments, the CNP agonist comprises a CNP molecule or moiety. In certain embodiments, the CNP agonist is CNP.

In certain embodiments, the CNP agonist comprises a CNP molecule or moiety having the sequence of SEQ ID NO:24, SEQ ID NO:25 or SEQ ID NO:30.

In certain embodiments, the CNP agonist is CNP having the sequence of SEQ ID NO:24, SEQ ID NO:25 or SEQ ID NO:30. In certain embodiments, the CNP agonist is CNP having the sequence of SEQ ID NO:24. In certain embodiments, the CNP agonist is CNP having the sequence of SEQ ID NO:25. In certain embodiments, the CNP agonist is CNP having the sequence of SEQ ID NO:30.

In certain embodiments, the liquid pharmaceutical formulation of the present invention may comprise one or more further excipients, such as for example, a stabilizer, anti-adsorption agent, cryoprotectant and other auxiliary agents. It is understood that one excipient may have multiple, such as dual or triple, functions.

In certain embodiments, the liquid pharmaceutical formulation of the present invention may comprise a stabilizer, such as a stabilizer selected from the group consisting of alanine; arginine; aspartic acid; glycine; histidine; lysine; proline; sugars such as glucose, sucrose, trehalose; polyols such as glycerol, mannitol, sorbitol; salts such as potassium phosphate, sodium sulphate; chelating agents such as EDTA, hexaphosphate; ligands such as divalent metal ions; other salts or organic molecules such as phenolic derivatives; oligomers or polymers such as cyclodextrins, dextran, dendrimers, PEG, PVP, protamine and HAS.

In certain embodiments, the liquid pharmaceutical formulation of the present invention may comprise an anti-adsorption agent, such as an anti-adsorption agent selected from the group consisting of ionic or non-ionic surfactants or other proteins or soluble polymers that are used to coat or adsorb competitively to the inner surface of the formulation or formulation's container such as poloxamer (Pluronic™ F-68), PEG dodecyl ether (Brij® 35), dextran, polyethylene glycol, PEG-polyhistidine, BSA, HAS and gelatines. Chosen concentration and type of excipient depends on the effect to be avoided but typically a monolayer of surfactant is formed at the interface just above the CMC.

In certain embodiments, the liquid pharmaceutical formulation of the present invention may comprise a cryoprotectant, such as a cryoprotectant selected from the group consisting of sugars, polyols, surfactants, amino acids, non-aqueous solvents and peptides. During freeze- or spray drying, cryoprotectants may counteract the destabilizing effects caused by hydrogen bond breaking and water removal. Trehalose is particularly efficient at reducing moisture-induced aggregation and also improves thermal stability potentially caused by exposure of the compound's hydrophobic groups to water. Mannitol and sucrose may also be used, either as sole lyo/cryoprotectant or in combination with each other where higher ratios of mannitol:sucrose are known to enhance physical stability of a compound in a liquid pharmaceutical formulation. Mannitol may also be combined with trehalose. Trehalose may also be combined with sorbitol or sorbitol may be used as the sole protectant. Starch or starch derivatives may also be used.

In certain embodiments, the liquid pharmaceutical formulation of the present invention may comprise further excipients selected from the group consisting of wetting agents, viscosity modifiers and antibiotics.

Applicant surprisingly found that within the liquid pharmaceutical formulation of the present invention the reversible linkage between CNP and the carrier is stable, eliminating the need of lyophilization and reconstitution from a lyophilizate. However, if desired, the liquid pharmaceutical formulation of the present invention may be dried, such as by lyophilization, to form a dry, such as a freeze-dried pharmaceutical formulation.

In certain embodiments, the liquid pharmaceutical formulation of the present invention comprises a CNP conjugate, acetic acid, mannitol, m-cresol and methionine.

In certain embodiments, the liquid pharmaceutical formulation of the present invention comprises a CNP conjugate, acetic acid, trehalose, m-cresol and methionine.

Due to high differences in patients' weight range, such as for example 3-80 kg, it is in certain embodiments beneficial to have liquid pharmaceutical formulations with different strengths, meaning different concentrations, of CNP conjugate, such as high, medium and low strengths. Thus, in one embodiment the liquid pharmaceutical formulation is provided in more than one concentration, such as in two different concentrations, such as in three different concentrations, such as in four different concentrations, such as in five different concentrations, such as in six different concentrations, such as in seven different concentrations, such as in eight different concentrations, such as in nine different concentrations, such as in ten different concentrations, such as in eleven different concentrations, such as in twelve different concentrations.

In certain embodiments, the present invention provides a liquid pharmaceutical formulation for storage for at least 6 months at 2 to 8° C.

In certain embodiments, the present invention provides a liquid pharmaceutical formulation for storage for at least 12 months at 2 to 8° C. In certain embodiments, the present invention provides a liquid pharmaceutical formulation for storage for at least 24 months at 2 to 8° C. In certain embodiments, the present invention provides a liquid pharmaceutical formulation for storage for at least 36 months at 2 to 8° C. In certain embodiments, the present invention provides a liquid pharmaceutical formulation for storage for at least 48 months at 2 to 8° C.

In certain embodiments, the present invention provides a liquid pharmaceutical formulation for storage for 6 to 48 months at 2 to 8° C. In certain embodiments, the present invention provides a liquid pharmaceutical formulation for storage for 6 to 36 months at 2 to 8° C. In certain embodiments, the present invention provides a liquid pharmaceutical formulation for storage for 6 to 24 months at 2 to 8° C. In certain embodiments, the present invention provides a liquid pharmaceutical formulation for storage for 6 to 12 months at 2 to 8° C.

In certain embodiments, the present invention provides a liquid pharmaceutical formulation for storage for one month at 25° C. In certain embodiments, the present invention provides a liquid pharmaceutical formulation for storage for one month at 30° C.

The liquid pharmaceutical formulation as described above is a liquid pharmaceutical formulation that is stable for at least 6 months, such as for at least 7 months, such as for at least 8 months, such as for at least 9 months, such as for at least 10 months, such as for at least 11 months, such as for at least 12 months. In certain embodiments, the liquid pharmaceutical formulation is stable for at least 14 months, such as for at least 16 months, such as for at least 18 months, such as for at least 20 months, such as for at least 22 months, such as for at least 24 months, such as for at least 36 months, such as for at least 48 months.

In certain embodiments, the liquid pharmaceutical formulation is stable for at least 36 months, when stored at 2° C. to 8° C. In certain embodiments, the liquid pharmaceutical formulation is stable for at least 36 months, when stored at 2° C. In certain embodiments, the liquid pharmaceutical formulation is stable for at least 6 months, when stored at 5° C. In certain embodiments, the liquid pharmaceutical formulation is stable for at least 2 weeks when stored at 30° C.

In certain embodiments, the liquid pharmaceutical formulation as described above can be stored at temperatures ranging from −80° C. up to 30° C., such as from −20° C. up to 25° C., such as from −15° C. up to 25° C., such as from −10° C. up to 25° C., such as from −5° C. up to 25° C., such as from 0° C. up to 25° C., such as from 2° C. to 8° C. In certain embodiments, the liquid pharmaceutical formulation is stored at 2° C. In certain embodiments, the liquid pharmaceutical formulation is stored at 4° C. In certain embodiments, the liquid pharmaceutical formulation is stored at 5° C. In certain embodiments, the liquid pharmaceutical formulation is stored at 8° C. In certain embodiments, the liquid pharmaceutical formulation is stored at 10° C. In certain embodiments, the liquid pharmaceutical formulation is stored at 16° C. In certain embodiments, the liquid pharmaceutical formulation is stored at 20° C. In certain embodiments, the liquid pharmaceutical formulation is stored at 25° C. In certain embodiments, the liquid pharmaceutical formulation is stored at 30° C. In certain embodiments, the liquid pharmaceutical formulation is stored at 40° C.

The liquid pharmaceutical formulation of the present invention comprises at least about 80%, such as at least 85%, 90%, 95%, 96%, 97%, 98% or 99% of the native form of the peptide (CNP or CNP moiety) after at least 3 months, 6 months, 12 months or at least 24 months of storage. Storage may take place at 5° C., 8° C., 15° C., 20° C. or 25° C. and the purity may be determined by any method known by the person skilled in the art, such as by SE-HPLC or RP-HPLC.

In certain embodiments, the liquid pharmaceutical formulation comprises about 90%, 95%, 96%, 97% or 99% of the native form of the peptide (CNP or CNP moiety) after 3 months or 6 months of storage at 5° C., as determined for example by SE-HPLC or RP-HPLC.

In certain embodiments, the liquid pharmaceutical formulation comprises about 80%, such as at least 85%, 90%, 95%, 96%, 97%, 98% or 99% of the native form of the peptide (CNP or CNP moiety) after 24 months of storage at 5° C., as determined by SE-HPLC or RP-HPLC.

In certain embodiments, the liquid pharmaceutical formulation comprises about 80%, 85%, 90% or 95% of the native form of the peptide (CNP or CNP moiety) after 6 months of storage at 25° C. as determined by SE-HPLC or RP-HPLC.

The present invention includes a method of manufacturing of a liquid pharmaceutical formulation according to the present invention comprising the steps of:
(i) admixing the CNP compound with at least a buffering agent, an isotonicity agent, a preservative and optionally an antioxidant;
(ii) adjusting the pH of the admixture of step (i);
(iii) optionally, filtering the admixture from step (ii);
(iv) transferring amounts of the admixture from step (ii) or (iii) equivalent to the desired number of dosages into a container;
(v) sealing the container; and wherein the order of steps (ii) and (iii) may optionally be reversed.

In certain embodiments, steps (ii) and (iii) are not reversed.

In certain embodiments, the CNP compound in step (i) is admixed with a buffering agent, an isotonicity agent, a preservative and optionally an antioxidant.

In certain embodiments, the method of manufacturing of a liquid pharmaceutical formulation according to the present invention comprises the steps of:
(i) admixing the CNP compound with at least acetic acid, mannitol, m-cresol and optionally an antioxidant;
(ii) adjusting the pH of the admixture of step (i);
(iii) optionally, filtering the admixture from step (ii);
(iv) transferring amounts of the admixture from step (ii) or (iii) equivalent to the desired number of dosages into a container;
(v) sealing the container; and
wherein the order of steps (ii) and (iii) may optionally be reversed.

In certain embodiments, steps (ii) and (iii) are not reversed.

In certain embodiments, the CNP compound in step (i) is admixed with acetic acid, mannitol, m-cresol and optionally an antioxidant.

In certain embodiments, the method of manufacturing of a liquid pharmaceutical formulation according to the present invention comprises the steps of:
(i) admixing a CNP conjugate with at least acetic acid, mannitol, m-cresol and optionally methionine to yield a formulation comprising:

| | | |
|---|---|---|
| CNP conjugate, of which CNP moiety | 0.1-20 | mg/ml |
| acetic acid | 0.3-3 | mg/ml |
| D-mannitol | 10-200 | mg/ml |
| m-cresol | 1-10 | mg/ml |

(ii) adjusting the pH of the admixture of step (i) to a pH ranging from pH 3.5 to pH 5.5 with NaOH and HCl;
(iii) optionally, filtering the admixture from step (ii);
(iv) transferring amounts of the admixture from step (ii) or (iii) equivalent to the desired number of dosages into a container;
(v) sealing the container; and
wherein the order of steps (ii) and (iii) may optionally be reversed.

In certain embodiments, the method of manufacturing of a liquid pharmaceutical formulation according to the present invention comprises the steps of:

(i) admixing a CNP conjugate with at least acetic acid, trehalose, m-cresol and optionally methionine to yield a formulation comprising:

| | | |
|---|---|---|
| CNP conjugate, of which CNP moiety | 0.1-20 | mg/ml |
| acetic acid | 0.3-3 | mg/ml |
| trehalose dihydrate | 10-200 | mg/ml |
| m-cresol | 1-10 | mg/ml. |

(ii) adjusting the pH of the admixture of step (i) to a pH ranging from pH 3.5 to pH 5.5 with NaOH and HCl;
(iii) optionally, filtering the admixture from step (ii);
(iv) transferring amounts of the admixture from step (ii) or (iii) equivalent to the desired number of dosages into a container;
(v) sealing the container; and
wherein the order of steps (ii) and (iii) may optionally be reversed.

In certain embodiments, steps (ii) and (iii) are not reversed.

Another aspect of the present invention refers to a container for the liquid pharmaceutical formulation of the present invention. The container may be suitable for both storing and dispensing the formulation. In certain embodiments, the container comprises the liquid pharmaceutical formulation of the present invention. When such a container is used the kit may additionally comprise a dispenser, such as a syringe or a receptacle with volumetric markings.

In certain embodiments, the container is made of glass. In certain embodiments, the internal surface of the container is coated with an inert material. In certain embodiments, the inert material is silicon.

In certain embodiments, the container may be selected from the group consisting of vial; syringe, such as dual-chamber syringe; ampoule and cartridge, such as dual-chamber cartridge.

In certain embodiments, the cartridge is for use with a pen injector, such as an auto-injector.

In certain embodiments, the liquid pharmaceutical formulation of the present invention is provided as a single dose, meaning that the container comprising the liquid pharmaceutical formulation comprises one therapeutic dose. It is clear to the skilled person that when the liquid pharmaceutical formulation is provided as a single dose, a preservative is not present in the formulation.

In certain embodiments, the liquid pharmaceutical formulation comprises multiple doses, meaning that the container comprising the liquid pharmaceutical formulation contains more than one therapeutic dose.

In certain embodiments, a multiple dose liquid pharmaceutical formulation comprises at least 2 doses, such as at least 4 doses, such as at least 6 doses, such as at least 8 doses, such as at least 10 doses, such as at least 12 doses of CNP agonist or CNP and in certain embodiments such as at least 14 doses.

In certain embodiments, a multiple dose liquid pharmaceutical formulation comprises at least 2, 4, 6, 8, 10, 12 or 14 doses of CNP agonist or CNP.

Thus, in another aspect of the present invention the liquid pharmaceutical formulation is provided as a multiple dose formulation.

A single dose formulation comprising the CNP compound, particularly of the CNP conjugate, has a volume of not more than 4 ml, such as from about 0.03 to about 1.1 ml. In certain embodiments, the volume is about 0.03 ml. In certain embodiments, the volume is about 0.05 ml. In certain embodiments, the volume is about 0.1 ml. In certain embodiments, the volume is about 0.3 ml. In certain embodiments, the volume is about 0.5 ml. In certain embodiments, the volume is about 0.8 ml. In certain embodiments, the volume is about 1 ml. In certain embodiments, the volume is about 1.5 ml. In certain embodiments, the volume is about 2 ml. In certain embodiments, the volume is about 3 ml. In certain embodiments, the volume is about 4 ml.

In certain embodiments, the CNP compound, particularly the CNP conjugate, is sufficiently dosed in the formulation to provide therapeutically effective amount of CNP for at least three days in one application, such as for at least four days, such as for at least five days, such as for at least six days.

In certain embodiments, the CNP compound, particularly the CNP conjugate is sufficiently dosed in the formulation to provide a therapeutically effective amount of CNP for one week.

The liquid pharmaceutical formulation of the present invention may be administered for example via topical, enteral or parenteral administration and by methods of external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection, infusion, intranasal, oral, transpulmonary and transdermal administration, direct delivery to the brain via implanted device allowing delivery of the invention or the like to brain tissue or brain fluids (e.g., Ommaya Reservoir), direct intracerebroventricular injection or infusion, injection or infusion into brain or brain associated regions, injection into the subchoroidal space, retro-orbital injection and ocular instillation, preferably via subcutaneous injection.

In certain embodiments, the liquid pharmaceutical formulation of the present invention is administered via subcutaneous injection.

In certain embodiments, the liquid pharmaceutical formulation of the present invention is administered via subcutaneous injection with a syringe and needle or a pen injector, such as an auto-injector.

In certain embodiments, the liquid pharmaceutical formulation of the present invention is administered via subcutaneous injection with a syringe and needle.

In certain embodiments, the liquid pharmaceutical formulation of the present invention is administered via subcutaneous injection with a pen injector.

In certain embodiments, the liquid pharmaceutical formulation of the present invention is administered via subcutaneous injection with an auto-injector.

The time period between two consecutive subcutaneous administrations, i.e. the administration interval, is in certain embodiments at least every 12 hours, 24 hours, 36 hours, 48 hours, 60 hours, 72 hours, 84 hours, 96 hours, 108 hours, 120 hours, 132 hours, 144 hours, 156 hours, one week, two weeks, three weeks or four weeks.

In certain embodiments, the time period between two consecutive subcutaneous administrations is 12 hours. In certain embodiments, the time period between two consecutive subcutaneous administrations is 24 hours. In certain embodiments, the time period between two consecutive subcutaneous administrations is 48 hours. In certain embodiments, the time period between two consecutive subcutaneous administrations is 72 hours. In certain embodiments, the time period between two consecutive subcutaneous administrations is 96 hours. In certain embodiments, the time period between two consecutive subcutaneous administrations is 120 hours. In certain embodiments, the time period between two consecutive subcutaneous administrations is 144 hours. In certain embodiments, the time period between two consecutive subcutaneous administrations is one week.

Another aspect of the present invention is the liquid pharmaceutical formulation of the present invention for use as a medicament.

In another aspect, the present invention relates to the liquid pharmaceutical formulation of the present invention for use in the treatment, control, delay or prevention of one or more diseases which can be treated, controlled, delayed or prevented with a CNP agonist, particularly CNP.

In certain embodiments, the present invention relates to the liquid pharmaceutical formulation of the present invention for use in the treatment of one or more diseases which can be treated with a CNP agonist, particularly CNP.

A further aspect of the present invention is a method of treating, controlling, delaying or preventing in a patient one or more diseases which can be treated by a CNP agonist, particularly CNP, the method comprising administering to the patient a therapeutically effective amount of the liquid pharmaceutical formulation of the present invention.

In certain embodiments, the patient is an adult. In certain embodiments, the patient is a pediatric patient.

The liquid pharmaceutical formulation of the present invention may be administered for at least six months, a year, five years, ten years, until a patient is 18 years old, until patient's epiphyseal closure or indefinitely. In certain embodiments, the liquid pharmaceutical formulation may be administered until a patient is 18 years old. In certain embodiments, the liquid pharmaceutical formulation may be administered until a patient's ephiphysis is closed.

In certain embodiments, said one or more diseases which can be treated, controlled, delayed or prevented with a CNP agonist, particularly CNP, are selected from the group consisting of bone-related disorders such as skeletal dysplasias; cancer; autoimmune diseases; fibrotic diseases; inflammatory diseases; central nervous system diseases such as neurodegenerative diseases; infectious diseases; lung diseases; heart and vascular diseases; metabolic diseases and ophthalmic diseases.

In certain embodiments, said one or more diseases which can be treated, controlled, delayed or prevented with a CNP agonist, particularly CNP, are selected from the group consisting of achondroplasia, hypochondroplasia, short stature, dwarfism, osteochondrodysplasias, thanatophoric dysplasia, osteogenesis imperfecta, achondrogenesis, chondrodysplasia punctata, homozygous achondroplasia, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphyseal dysplasia, neurofibromatosis, LEOPARD syndrome, Noonan syndrome, hereditary gingival fibromatosis, neurofibromatosis type 1, Legius syndrome, cardiofaciocutaneous syndrome, Costello syndrome, SHOX deficiency, idiopathic short stature, growth hormone deficiency, osteoarthritis, cleidocranial dysostosis, craniosynostosis (e.g., Muenke syndrome, Crouzon syndrome, Apert syndrome, Jackson-Weiss syndrome, Pfeiffer syndrome, or Crouzonodermoskeletal syndrome), dactyly, brachydactyly, camptodactyly, polydactyly, syndactyly, dyssegmental dysplasia, enchondromatosis, fibrous dysplasia, hereditary multiple exostoses, hypophosphatemic rickets, Jaffe-Lichtenstein syndrome, Marfan syndrome, McCune-Albright syndrome, osteopetrosis, osteopoikilosis, hemorrhagic shock, hypertension, restenosis, arteriosclerosis, acute decompensated heart failure, congestive heart failure, cardiac edema, nephredema, hepatic edema, acute renal insufficiency, chronic renal insufficiency, glaucoma, elevated intraocular pressure, multiple myeloma, myeloproliferative syndrome, leukemia, plasma cell leukemia, lymphoma, glioblastoma, prostate cancer, bladder cancer, mammary cancer, growth retardation, skull deformities, orthodontic defects, cervical cord compression, spinal stenosis, hydrocephalus, hearing loss due to chronic otitis, cardiovascular disease, neurological disease and obesity.

In certain embodiments, said one or more diseases which can be treated, controlled, delayed or prevented with a CNP agonist, particularly CNP, are selected from the group consisting of achondroplasia such as homozygous achondroplasia, hypochondroplasia, short stature, dwarfism, osteochondrodysplasias, thanatophoric dysplasia, osteogenesis imperfecta, achondrogenesis, chondrodysplasia punctata, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphyseal dysplasia, neurofibromatosis, Legius syndrome, LEOPARD syndrome, Noonan syndrome, hereditary gingival fibromatosis, neurofibromatosis type 1, Legius syndrome, cardiofaciocutaneous syndrome, Costello syndrome, SHOX deficiency, idiopathic short stature, growth hormone deficiency, osteoarthritis, cleidocranial dysostosis, craniosynostosis (e.g., Muenke syndrome, Crouzon syndrome, Apert syndrome, Jackson-Weiss syndrome, Pfeiffer syndrome, or Crouzonodermoskeletal syndrome), dactyly, brachydactyly, camptodactyly, polydactyly, syndactyly, dyssegmental dysplasia, enchondromatosis, fibrous dysplasia, hereditary multiple exostoses, hypophosphatemic rickets, Jaffe-Lichtenstein syndrome, Marfan syndrome, McCune-Albright syndrome, osteopetrosis, osteopoikilosis, hemorrhagic shock, hypertension, restenosis, arteriosclerosis, acute decompensated heart failure, congestive heart failure, cardiac edema, nephredema, hepatic edema, acute renal insufficiency, chronic renal insufficiency, glaucoma, elevated intraocular pressure, multiple myeloma, myeloproliferative syndrome, leukemia, plasma cell leukemia, lymphoma, glioblastoma, prostate cancer, bladder cancer, mammary cancer, growth retardation, skull deformities, orthodontic defects, cervical cord compression, spinal stenosis, hydrocephalus, hearing loss due to chronic otitis, obesity, disorders involving abnormal RAS-mitogen-activated protein kinase signaling, pulmonary hypertension, vasculopathy, endothelial dysfunction, liver cirrhosis, liver ascites, liver fibrosis, hepatorenal syndrome, asthma, pulmonary fibrosis, chronic kidney diseases, cardiorenal syndrome, dyspnea and lysosomal storage diseases such as mucopolysaccharidosis.

In certain embodiments, said one or more diseases which can be treated, controlled, delayed or prevented with a CNP agonist, particularly CNP, is one or more cardiovascular diseases selected from the group consisting of arrhythmia such as cardiac or sinus arrhythmia; atrial fibrillation; atrial flutter; bradycardia; Brugada syndrome; premature cardiac complexes; commotio cordis; heart block; long QT syndrome; parasystole; pre-excitation syndrome; tachycardia; ventricular fibrillation; ventricular flutter; cardiac conduction system disease; low cardiac output; cardiomegaly; dilated cardiomyopathy; hypertrophy such as left ventricular hypertrophy or right ventricular hyperthrophy; cardiomyopathy such as alcoholic, dilated, hypertrophic, restrictive, diabetic or Chagas cardiomyopathy; arrhythmogenic right ventricular dysplasia; endocardial fibroelastosis; endomyocardial fibrosis; glycogen storage disease type IIb; Kearns-Sayre syndrome; myocardial reperfusion injury; myocarditis; sarcoglycanophaties; endocarditis such as bacterial or non-infective endocarditis; heart arrest; sudden cardiac death; out-of-hospital cardiac arrest; cardio-renal syndrome; paroxysmal dyspnea; cardiac edema, heart failure such as diastolic or systolic heart failure; heart valve disease; aortic valve insufficiency; aortic valve stenosis; heart valve prolapse; mitral valve insufficiency; mitral valve stenosis; pulmonary atresia; pulmonary valve insufficiency; pulmonary valve stenosis; tricuspid atresia; tricuspid valve insufficiency; tricuspid valve stenosis; myocardial ischemia; acute coronary syndrome; angina pectoris; coronary disease; Kounis syndrome; myocardial infarction; pulmonary heart disease; ventricular dysfunction such as left or right ventricular dysfunction; ventricular outflow obstruction; aortic valve stenosis, pulmonary valve stenosis; hypertension; atherosclerosis; restenosis; critical limb ischaemia; peripheral arterial disease; ischemia such as ischemia-reperfusion injury or ischemic injuries; abnormal fluid accumulation in the heart and myocardial edema.

In certain embodiments, said one or more diseases which can be treated, controlled, delayed or prevented with a CNP agonist, particularly CNP, is selected from the group consisting of ischemic heart disease such as myocardial infarction; congestive heart failure; arrhythmia and atherosclerosis.

In certain embodiments, said one or more diseases which can be treated, controlled, delayed or prevented with a CNP agonist, particularly CNP, is one or more central nervous system diseases selected from the group consisting of brain ischemia such as ischemic hypoxia; brain infarction; transient ischemic attack; vertebrobasilar insufficiency; cerebrovascular disorders; stroke; intracranial hemorrhages; corneal neovascularization; corneal transplantation; gragft-versus-host disease; graft rejection; glaucoma such as angle-closure, neovascular, open-angle or low tension glaucoma; ischemic optic neuropathy; central serous chorioretinopathy; retinopathy such as diabetic or hypertensive retinopathy; retinal degeneration; macular degeneration; geographic atrophy; macular edema; Stargardt disease; vitelliform macular dystrophy; wet macular degeneration; retinoschisis; retinal detachment; retinal perforations; retinal haemorrhage; retinal neovascularization; retinal vein occlusion; retinal artery occlusion; retinopathy of prematurity and proliferative vitreoretinopathy.

In certain embodiments, said one or more diseases are selected from the group consisting of hypophosphatasia, hypochondroplasia, Muenke syndrome, hypertension, osteogenesis imperfecta and achondroplasia.

In certain embodiments, said one or more diseases which can be treated with a CNP agonist, particularly CNP, is hypophosphatasia. In certain embodiments, said one or more diseases which can be treated with CNP is hypochondroplasia. In certain embodiments, said one or more diseases which can be treated with CNP is Muenke syndrome. In certain embodiments, said one or more diseases which can be treated with CNP is hypertension. In certain embodiments, said one or more diseases which can be treated with CNP is osteogenesis imperfecta. In certain embodiments, said one or more diseases which can be treated with CNP is achondroplasia.

The invention is further described by the following non-limiting items.
1. A liquid pharmaceutical formulation comprising a CNP compound, a buffering agent, an isotonicity agent, a preservative and optionally an antioxidant.
2. The liquid pharmaceutical formulation of item 1 for storage for at least 6 months at 2 to 8° C.
3. The liquid pharmaceutical formulation of item 1 or 2 for storage for 6 to 48 months at 2 to 8° C.
4. The liquid pharmaceutical formulation of any one of items 1 to 3 for storage for 6 to 36 months at 2 to 8° C.
5. The liquid pharmaceutical formulation of any one of items 1 to 4 for storage for 6 to 36 months at 5° C.
6. The liquid pharmaceutical formulation of any one of items 1 to 5, wherein the CNP compound is a CNP conjugate.
7. The liquid pharmaceutical formulation of any one of items 1 to 6, wherein the buffering agent is selected from the group consisting of acetic acid, succinic acid, citric acid, lactic acid, glutamic acid, fumaric acid, aspartic acid, glutaric acid, phosphoric acid, histidine, gluconic acid, tartaric acid, malic acid and Tris (tris (hydroxymethyl) aminomethane).
8. The liquid pharmaceutical formulation of any one of items 1 to 7, wherein the buffering agent is acetic acid.
9. The liquid pharmaceutical formulation of any one of items 1 to 8, wherein the isotonicity agent is selected from the group consisting of mannitol, trehalose, sucrose, raffinose, gelatin, lactose, dibasic calcium phosphate, sorbitol, xylitol, glycine, histidine, ethanol, hydroxyethylstarch, potassium chloride, sodium chloride, dextrose, dextran, propylene glycol and glycerol.
10. The liquid pharmaceutical formulation of any one of items 1 to 9, wherein the isotonicity agent is mannitol or trehalose.
11. The liquid pharmaceutical formulation of any one of items 1 to 10, wherein the preservative is selected from the group consisting of m-cresol, benzyl alcohol, benzoic acid, phenol, methylparaben, ethylparaben, propylparaben, butylparaben, potassium sorbate, chlorobutanol, benzyl alcohol, phenylmercuric nitrate, thimerosal, sorbic acid, potassium sorbate, chlorocresol, benzalkonium chloride, 2-ethoxyethanol, chlorhexidine, chlorobutanol, phenylethyl alcohol and phenylmercuric acetate.
12. The liquid pharmaceutical formulation of any one of items 1 to 11, wherein the preservative is m-cresol or phenol.
13. The liquid pharmaceutical formulation of any one of items 1 to 12, wherein the pharmaceutical formulation comprises an antioxidant selected from the group consisting of methionine, butylhydroxytoluene, butylhydroxyanisol, tocopherol, propylgallate, ascorbic acid, sodium bisulfite, ethylenediaminetetraacetic acid (EDTA), cysteine, glutathione, monothioglycerol, poly(ethylenimine), vitamin E, ectoine and morin.
14. The liquid pharmaceutical formulation of any one of items 1 to 13, wherein the pH of the liquid formulation is from about pH 3.5 to about pH 5.5.
15. The liquid pharmaceutical formulation of any one of items 1 to 14, wherein the pH of the liquid formulation is about 4.5.
16. The liquid pharmaceutical formulation of any one of items 1 to 15, wherein the liquid formulation further comprises a pH-adjusting agent.
17. The liquid pharmaceutical formulation of item 16, wherein the pH-adjusting agent is selected from the group consisting of sodium hydroxide, sodium acetate, Tris (tris (hydroxymethyl) aminomethane), potassium hydroxide, lysine, and mixtures thereof.
18. The liquid pharmaceutical formulation of any one of items 1 to 17, wherein the liquid pharmaceutical formulation comprises a CNP compound, acetic acid, an isotonicity agent, a preservative and optionally an antioxidant.
19. The liquid pharmaceutical formulation of any one of items 1 to 18, wherein the liquid pharmaceutical formulation comprises a CNP compound, acetic acid, trehalose dihydrate, a preservative and optionally an antioxidant.
20. The liquid pharmaceutical formulation of any one of items 1 to 19, wherein the liquid pharmaceutical formulation comprises a CNP compound, acetic acid, trehalose dihydrate, m-cresol and optionally an antioxidant.
21. The liquid pharmaceutical formulation of any one of items 1 to 20, wherein the liquid pharmaceutical formulation comprises a CNP compound, acetic acid, trehalose dihydrate, m-cresol and methionine.
22. The liquid pharmaceutical formulation of any one of items 1 to 17, wherein the liquid pharmaceutical formulation comprises a CNP compound, acetic acid, mannitol, a preservative and optionally an antioxidant.
23. The liquid pharmaceutical formulation of any one of items 1 to 17 or 22, wherein the liquid pharmaceutical formulation comprises a CNP compound, acetic acid, mannitol, m-cresol and optionally an antioxidant.
24. The liquid pharmaceutical formulation of any one of items 1 to 17, 22 or 23, wherein the liquid pharmaceutical formulation comprises a CNP compound, acetic acid, mannitol, m-cresol and methionine.
25. The liquid pharmaceutical formulation of any one of items 1 to 24, wherein the CNP compound is a reversible CNP conjugate that comprises a CNP moiety that is covalently and reversibly conjugated to a carrier moiety.
26. The liquid pharmaceutical formulation of item 25, wherein the CNP moiety has the sequence of SEQ ID NO:9, SEQ ID NO:10, SEQ ID NO:11, SEQ ID NO:12, SEQ ID NO: 13, SEQ ID NO:14, SEQ ID NO:15, SEQ ID NO:16, SEQ ID NO:17, SEQ ID NO: 18, SEQ ID NO:19, SEQ ID NO:20, SEQ ID NO:21, SEQ ID NO:22, SEQ ID NO: 23, SEQ ID NO:24, SEQ ID NO:25 or SEQ ID NO:30.
27. The liquid pharmaceutical formulation of item 25 or 26, wherein the CNP moiety has the sequence of SEQ ID NO:20, SEQ ID NO:21, SEQ ID NO:22, SEQ ID NO:23, SEQ ID NO: 24, SEQ ID NO:25 or SEQ ID NO:30.
28. The liquid pharmaceutical formulation of any one of items 25 to 27, wherein the CNP moiety has the sequence of SEQ ID NO:24.
29. The liquid pharmaceutical formulation of any one of items 25 to 28, wherein the carrier moiety is a polymeric moiety.
30. The liquid pharmaceutical formulation of any one of items 1 to 29, wherein the CNP compound is a reversible CNP conjugate of formula (Ia):

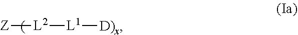

wherein
-D is a CNP moiety;
-L¹- is a reversible linker moiety;
-L²- is a single chemical bond or a spacer moiety;
—Z is a carrier moiety; and
x is an integer selected from the group consisting of 1, 2, 3, 4, 5, 6, 7, 8, 9, 10, 11, 12, 13, 14, 15 and 16.
31. The liquid pharmaceutical formulation of item 30, wherein x of formula (Ia) is 1.
32. The liquid pharmaceutical formulation of item 30 or 31, wherein-Z is a branched polymeric moiety.
33. The liquid pharmaceutical formulation of any one of items 30 to 32, wherein-Z is a branched PEG-based polymeric moiety.
34. The liquid pharmaceutical formulation of any one of items 6 to 18 or 22 to 33, wherein the liquid pharmaceutical formulation comprises:

| | | |
|---|---|---|
| CNP conjugate, of which CNP moiety | 0.1-20 | mg/ml |
| acetic acid | 0.3-3 | mg/ml |
| D-mannitol | 10-200 | mg/ml |
| m-cresol | 1-10 | mg/ml. |

35. The liquid pharmaceutical formulation of any one of items 6 to 21 or 25 to 33, wherein the liquid pharmaceutical formulation comprises:

| | | |
|---|---|---|
| CNP conjugate, of which CNP moiety | 0.1-20 | mg/ml |
| acetic acid | 0.3-3 | mg/ml |
| trehalose dihydrate | 10-200 | mg/ml |
| m-cresol | 1-10 | mg/ml. |

36. The liquid pharmaceutical formulation of any one of items 30 to 35, wherein the moiety -L¹- is of formula (II):

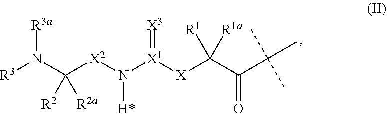

wherein the dashed line indicates the attachment to a nitrogen of -D which is a CNP moiety by forming an amide bond;
—X— is —C(R⁴R^{4a})—, —N(R⁴)—, —O—, —C(R⁴R^{4a})—C(R⁵R^{5a})—, —C(R⁵R^{5a})—C(R⁴R^{4a})—, —C(R⁴R^{4a})—N(R⁶)—, —N(R⁶)—C(R⁴R^{4a})—, —C(R⁴R^{4a})—O—, —O—C(R⁴R^{4a})— or —C(R⁷R^{7a})—;
X¹ is C or S (O);
—X²— is —C(R⁸R^{8a})— or —C(R⁸R^{8a})—C(R⁹R^{9a})—;
═X³ is ═O, ═S or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H and C₁₋₆ alkyl, provided that in case one of —R³, —R^{3a} or both are other than —H they are connected to the N atom to which they are attached through an sp³-hybridized carbon atom;
—R⁷ is-N(R¹⁰R^{10a}); or —NR¹⁰—(C═O)—R¹¹;
—R^{7a}, —R¹⁰, —R^{10a}, —R¹¹ are independently of each other-H; or C₁₋₆ alkyl;
optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}—R⁸/—R^{8a}, —R⁹/—R^{9a} are joined together with the atom to which they are attached to form a C₃₋₁₀ cycloalkyl; or 3- to 10-membered heterocyclyl;
   optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹, —R²/—R³ are joined together with the atoms to which they are attached to form a ring A;
   optionally, —R³/—R^{3a} are joined together with the nitrogen atom to which they are attached to form a 3- to 10-membered heterocycle;
   A is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; and
   wherein -L¹- is substituted with -L²-Z and wherein -L¹- is optionally further substituted, provided that the hydrogen marked with the asterisk in formula (II) is not replaced by -L²-Z or a substituent.
37. The liquid pharmaceutical formulation of any one of items 30 to 36, wherein the moiety -L¹-L²- is of formula (IId-ii):

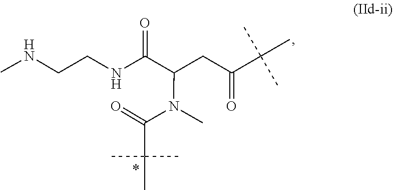

wherein
the unmarked dashed line indicates attachment to a nitrogen of an amino acid side chain of the ring moiety of -D by forming an amide bond; and the dashed line marked with the asterisk indicates attachment to —Z; wherein-D has the sequence of SEQ ID NO:24 and -L¹- is conjugated to the lysine at position 26;
wherein —Z is of formula (h):

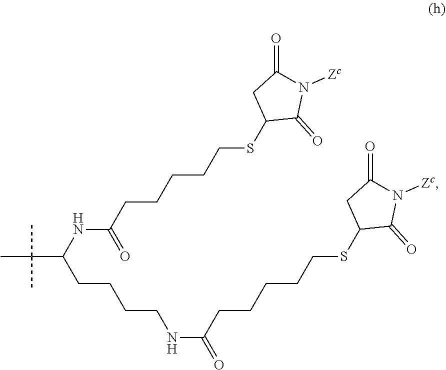

wherein
the dashed line indicates attachment to -L²-; and
each —Z^{c} is a moiety

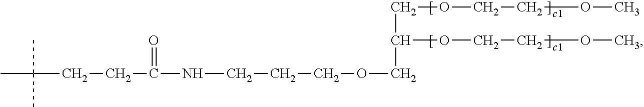

wherein
each c1 is an integer independently ranging from about 200 to 250.
38. The liquid pharmaceutical formulation of any one of items 30 to 37, wherein the moiety -L¹-L²- is of formula (IId-ii′):

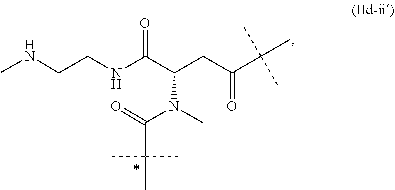

wherein
the unmarked dashed line indicates attachment to -D as defined in item 37 and the marked dashed line indicates attachment to —Z as defined in item 37.
39. The liquid pharmaceutical formulation of of any one of items 30 to 38, wherein the moiety -L¹-L²- is of formula (h-i):

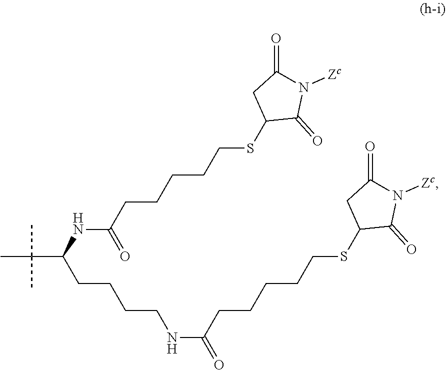

wherein
the dashed line indicates attachment to -L²-; and
each —Z^{c} is a moiety

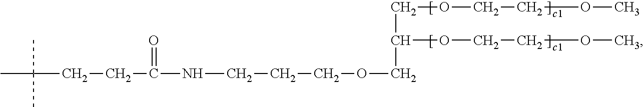

wherein
each c1 is an integer independently ranging from 200 to 250.
40. The liquid pharmaceutical formulation of any one of items 37 to 39, wherein each c1 is about 225.
41. The liquid pharmaceutical formulation of any one of items 1 to 24, 34 or 35, wherein the CNP compound is a stable CNP conjugate that comprises a CNP moiety that is covalently and stably conjugated to a carrier moiety.
42. The liquid pharmaceutical formulation of any one of items 1 to 24, 34, 35 or 41, wherein the CNP compound is a stable CNP conjugate that comprises a CNP moiety that is covalently and stably conjugated to a fatty acid derivative.
43. The liquid pharmaceutical formulation of any one of items 1 to 17 or 23 to 29, wherein the CNP compound is a CNP agonist.
44. The liquid pharmaceutical formulation of item 43, wherein the CNP agonist is CNP.
45. The liquid pharmaceutical formulation of item 44, wherein CNP has the sequence of SEQ ID NO:9, SEQ ID NO: 10, SEQ ID NO:11, SEQ ID NO: 12, SEQ ID NO:13, SEQ ID NO: 14, SEQ ID NO:15, SEQ ID NO: 16, SEQ ID NO: 17, SEQ ID NO:18, SEQ ID NO: 19, SEQ ID NO:20, SEQ ID NO:21, SEQ ID NO:22, SEQ ID NO:23, SEQ ID NO: 24, SEQ ID NO:25 or SEQ ID NO:30.
46. The liquid pharmaceutical formulation of item 44 or 45, wherein CNP has the sequence of SEQ ID NO:20, SEQ ID NO:21, SEQ ID NO:22, SEQ ID NO:23, SEQ ID NO:24, SEQ ID NO:25 or SEQ ID NO:30.
47. A method of manufacturing the liquid pharmaceutical formulation of any one of items 1 to 46, wherein the method comprises the following steps:
   (i) admixing the CNP compound of any one of items 1 to 46 with at least a buffering agent, an isotonicity agent, a preservative and optionally an antioxidant;
   (ii) adjusting the pH of the admixture of step (i);
   (iii) optionally, filtering the admixture from step (ii);
   (iv) transferring amounts of the admixture from step (ii) or (iii) equivalent to the desired number of dosages into a container;
   (v) sealing the container; and
   wherein the order of steps (ii) and (iii) may optionally be reversed.
48. The method of item 47, wherein the buffering agent is acetic acid.
49. The method of item 47 or 48, wherein the isotonicity agent is selected from the group consisting of mannitol and trehalose.
50. A container comprising the liquid pharmaceutical formulation of any one of items 1 to 46.
51. The container of item 50, wherein the container is selected from the group consisting of vial, syringe, ampoule and cartridge.
52. The liquid pharmaceutical formulation of any one of items 1 to 46, for use in the treatment of one or more diseases which can be treated with CNP.
53. The liquid pharmaceutical formulation for use of item 52, wherein the disease which can be treated with CNP is selected from the group consisting of achondroplasia, hypochondroplasia, short stature, dwarfism, osteochondrodysplasias, thanatophoric dysplasia, osteogenesis imperfecta, achondrogenesis, chondrodysplasia punctata, homozygous achondroplasia, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatropic dysplasia, spondyloepimetaphyseal dysplasia, neurofibromatosis, Legius syndrome, LEOPARD syndrome, Noonan syndrome, hereditary gingival fibromatosis, neurofibromatosis type 1, cardiofaciocutaneous syndrome, Costello syndrome, SHOX deficiency, idiopathic short stature, growth hormone deficiency, osteoarthritis, cleidocranial dysostosis, craniosynostosis, dactyly, brachydactyly, camptodactyly, polydactyly, syndactyly, dyssegmental dysplasia, enchondromatosis, fibrous dysplasia, hereditary multiple exostoses, hypophosphatemic rickets, Jaffe-Lichtenstein syndrome, Marfan syndrome, McCune-Albright syndrome, osteopetrosis and osteopoikilosis.
54. The liquid pharmaceutical formulation for use of item 52, wherein the disease which can be treated with CNP is selected from the group consisting of achondroplasia, hypochondroplasia, short stature, Noonan syndrome and SHOX deficiency.
55. The liquid pharmaceutical formulation for use of item 52, wherein the disease is achondroplasia.
56. A method of treating, reducing the risk of or delaying in a human patient a disease treatable by CNP, the method comprising the step of administering to said patient of a therapeutically effective amount of the liquid pharmaceutical formulation of any one of items 1 to 46.
57. The method of item 56, wherein the disease is selected from the group consisting of achondroplasia, hypochondroplasia, short stature, Noonan syndrome and SHOX deficiency.
58. The method of item 56 or 57, wherein the disease is achondroplasia.
59. The liquid pharmaceutical formulation of any one of items 1 to 46 for use in the manufacture of a medicament for the treatment of achondroplasia.

### EXAMPLES

### Materials and Methods

Peptides: CNP-38 TFA salt, CNP-27 TFA salt, CNP-53 acetate salt, PG-CNP-37 TFA salt were obtained from Caslo, Lyngby, Denmark. Peptide mapping reference peptides were obtained from Wuxi, China.

Reagents: TFA (sequencing grade, Thermo), water (ULC/MS grade, Biosolve and hypotonic, sterile, free from pyrogen, Carl Roth), acetonitrile (ULC/MS grade, Biosolve), Poloxamer 188 (Cell culture grade, Applichem), Tween-20 (Pharm. Eur, Carl Roth), Thermolysin (Promega), NaH₂PO₄*H₂O (ACS >98%, Sigma), 4 M NaOH (Carl Roth), 1 M NaOH (Reag. Ph. Eur., Carl Roth), 1 M HCl (Reag. Ph. Eur., Carl Roth), NaCl (Pharm. Eur. >99%, Carl Roth), Phosphoric acid (ACS grade, >85%, Merck), CaCl₂) dihydrate (ACS grade >99%), trehalose dihydrate (USP grade, Pfanstiehl), succinic acid (for analysis, ACS, Applichem), acetic acid (ROTIPURAN 100%, p.a., Carl Roth and Ph Eur, BP, JP, USP, ACS, Merck), metacresol (Ph.Eur./USP parenteral grade, Hedinger), phenol (puriss. p.a., ACS reagent, reag. Ph. Eur., VWR) mannitol (Ph Eur, BP, USP, JP, Merck and Ph. Eur., USP, BP., Carl Roth), citric acid (ACS reagent, >99.5%, Sigma), citric acid monohydrate (Ph Eur, BP, ChP, JP, USP, E 330, FCC), sodium citrate dihydrate (>99%, Ph. Eur., Carl Roth) EDTA disodium salt dihydrate (suitable for electrophoresis, for molecular biology, 99.0-101% [titration], Sigma), methionine (Ph. Eur., USP, AppliChem), tris (BioPerformance Certified, meets EP, USP testing specifications, suitable for cell culture, >99.9% [titration], Sigma), Polysorbate 80 (tested according to Ph. Eur., Sigma Aldrich).

### Preparation of CNP-38 Formulations (F1-F23)

Excipient stock solutions were prepared by combining the required excipients in a volumetric flask and filling it up to 90% of the desired volume with water. The pH values of the stock solutions were adjusted using either 1 M Tris solution or 4 M NaOH solution as required and the volume adjusted to the desired volume with water. The stock solutions were filtered through a 0.2 μm PVDF-filter. Phenol and m-cresol were spiked into the formulations as needed. Next, the required amount of CNP-38 was dissolved in the required amount of the desired excipient stock solution and the pH was checked and adjusted if needed with either 1.0 M Tris or 0.1 M NaOH as necessary. The formulations were filtered through a 0.22 μm PVDF-filter, aliquoted ready for incubation.

### Preparation of CNP Formulations (F33-45)

All excipient stock solutions, except for phenol and m-cresol, were prepared by combining the required excipients in a volumetric flask and filling it up to 90% of the desired volume with water. The pH of the stock solutions was adjusted using 1 M NaOH solution as required and the volume adjusted to the desired volume with water. The stock solutions were filtered through a 0.2 μm PVDF-filter. Stock solutions of phenol (1.75%) and m-cresol (1.05%) were prepared by adding the preservative to the respective excipient stock solution after filtration. Next, the required amount of CNP was dissolved in the required amount of the desired excipient stock solution and the pH was checked and adjusted if needed with 1 M NaOH. The formulations were filtered through a 0.22 μm PVDF filter and the respective preservative stock solution was added. The formulations were aliquoted ready for incubation.

### Preparation of Compound (1) Formulations (F24-F32)

A solution of compound (1) was buffer exchanged by using diafiltration in the desired buffer and then concentrated by ultrafiltration. Diafiltration and ultrafiltration were carried out by TFF using an Äkta Flux S TFF system and a Pellicon XL 50 Cassette (Biomax 10 kDa). After ultrafiltration, the concentrations were checked, and the solutions diluted back to a concentration of either 3.6 or 6.0 mg CNP-38 eq./ml. Next, the stock solutions were aliquoted, spiked with methionine where required and the pH checked and adjusted where necessary. Finally, the samples were spiked with m-cresol, sterile filtered using a 0.22 μm PVDF filter and aliquoted ready for incubation.

### Preparation of Compound (1) Formulations F46-F50:

A solution of compound (1) was buffer exchanged by using diafiltration into the designated buffer and then concentrated by ultrafiltration. Diafiltration and ultrafiltration were carried out using ultracentrifugal filters (Amicon® Ultra-15, Ultracel®-10 K). After ultrafiltration, the concentrations were checked, and the solutions diluted back to a concentration of 2.8 mg CNP-38 eq./ml. Next, the pH was checked and adjusted where necessary with 1 M HCl. The formulations were filtered using a 0.22 μm PVDF filter, the preservative stock solutions were added and by that the samples were diluted to 2.2 mg CNP-38 eq./ml. The formulations were aliquoted ready for incubation.

pH: The pH values of the formulations were measured at room temperature with a calibrated pH meter using a normal ionic strength electrode.

### Analytical Methods

RP-HPLC-MS to determine the purity of CNP-38: Mobile phase A was composed of 0.05% aqueous TFA (v/v) and mobile phase B was composed of 0.04% TFA (v/v) in acetonitrile. A Waters Acquity CSH C18, 130 Å, 1.7 μm, 2.1×100 mm column was used. Flow rate was set to 0.3 ml/min, detection was at a wavelength of 215 nm, the column running temperature was 60° C. (+1° C.). For LCMS the HPLC system was coupled to an LTQ Orbitrap Discovery XL instrument and ESI-MS was performed in the positive mode. A stepwise elution gradient was used and is shown in Table 21.

TABLE 21

| Time [min] | % B |
|---|---|
| 0.00 | 14.0 |
| 17.00 | 18.0 |
| 19.00 | 18.0 |
| 26.00 | 47.0 |
| 32.00 | 56.0 |
| 34.00 | 56.0 |
| 39.00 | 90.0 |
| 41.00 | 90.0 |
| 42.00 | 14.0 |
| 52.00 | 14.0 |

Preparative RP-HPLC to isolate succinate adduct CNP-38. Mobile phase A was composed of water/acetonitrile/TFA (95/5/0.05, v/v/v) and mobile phase B was composed of water/acetonitrile/TFA (75/25/0.05, v/v/v). A Waters Acquity CSH C18, 130 Å, 10 μm, 10×150 mm column (+10×10 mm pre-column) was used. Flow rate was set to 10 ml/min, detection was at a wavelength of 215 nm, the column running temperature room temperature The elution gradient is shown in Table 22.

TABLE 22

| Time [min] | % B |
|---|---|
| 0.0 | 50 |
| 2.5 | 60 |
| 17.5 | 60 |
| 27.5 | 67.5 |
| 30 | 75 |
| 35 | 100 |

RP-HPLC to determine the content and purity of compound (1) and to detect free CNP-38: Mobile phase A was composed of 0.05% aqueous TFA (v/v) and mobile phase B was composed of 0.04% TFA (v/v) in acetonitrile. A Waters Acquity CSH C18, 130 Å, 1.7 μm, 2.1×100 mm column was used. Flow rate was set to 0.3 ml/min, detection was at a wavelength of 215 nm, the column running temperature was 60° C. (+1° C.) and the sample injection load was approx. 3 μg CNP-38 eq. (20 μL of a 0.135 mg/ml sample). A stepwise eluent gradient running first from 5% B to 54% B over 25.5 min and then from 54% B to 59% B over 10.0 min was used. Samples were diluted to 0.15 mg CNP-38 eq./ml with dilution buffer (10 mM succinic acid, 84 g/L trehalose dihydrate, adjusted to pH 5 with 1 M Tris) and further diluted 9:1 (w/w) with dilution buffer containing 0.5% Tween-20 (w/v). The content was determined by peak area comparison to a reference solution with known content. Free CNP-38 was identified by reference peptide and quantified relative to the total peak area.

SE-HPLC to determine the purity of (1): Mobile phase was composed of 15 mM sodium phosphate pH 7.40, 135 mM sodium chloride, 0.2% Poloxamer 188 in water (e.g. 2.07 g NaH₂PO₄*H₂O, 7.89 g NaCl and 2.00 g Poloxamer 188 in 1 L HPLC-grade water; pH adjusted to 7.40 using 4 M aqueous NaOH solution). A GE Superdex 200 Increase 10/300 GL column was used. Isocratic flow rate was set to 0.75 ml/min, detection was at a wavelength of 215 nm, the column running temperature was room temperature and the sample injection load was 17 μg CNP-38 eq. (50 μL of a 0.34 mg/ml sample). Samples were diluted to 0.34 mg/mL with dilution buffer (10 mM succinic acid, 84 g/l trehalose dihydrate, adjusted to pH 5 with 1 M Tris) and dilution buffer containing 0.1% Poloxamer 188 (w/v) so that the final concentration of Poloxamer 188 was 0.05% (e.g. 20 μL of a 3.6 mg CNP-38 eq./ml solution were diluted with 86 μL dilution buffer to 0.68 mg/ml and 50 μL of this solution were further diluted with 50 μL dilution buffer containing 0.1% Poloxamer 188).

Peptide mapping of (1) to assess aspartate (28) conversion into isoaspartate (28) (Isoasp) and methionine (33) oxidation into methionine sulfoxide (33) (Met (O)): Thermolysin digestion of (1) was performed at a thermolysin/CNP-38 ratio=1:20 (w/w). The pH was adjusted with proteolysis buffer concentrate to pH 7.5. After 7 h incubation at 37° C. the digest was stopped by the addition of 5% (v/v) phosphoric acid. The resulting peptide mixture was separated by RP-HPLC on Waters Acquity UPLC HSS T3, 100 Å, 1.8 μm, 2.1×150 mm column with 0.10 vol % aq. TFA (v/v) as mobile phase A and 0.09% TFA (v/v) in acetonitrile as mobile phase B and detection at 210 nm. Flow rate was set to 0.28 ml/min, the column running temperature was 45° C. (+1° C.) and the sample injection load was 19 μg CNP-38 eq. (e.g. 20 μL of a 0.94 mg/ml sample). An eluent gradient running from 0% B to 3% B over 31.0 min was used. The thermolysin fragments which contain aspartate (28) (LisoDR) and methionine (33) (IGSM (O) SG) were identified by reference peptides and quantified based on their respective peak areas relative to the peak areas of the corresponding unmodified fragments (LDR; IGS+MSG).

Peptide mapping with LCMS for CNP-38 and variants. Peptide Mapping and RP-HPLC was done as described for (1). For LCMS the HPLC system was coupled to an LTQ Orbitrap Discovery XL instrument and ESI-MS was performed in positive mode.

IEX-HPLC Mobile phase A was composed of 20 mM sodium phosphate buffer pH 6.5 containing 10% (v/v) ethanol and mobile phase B was composed of 20 mM sodium phosphate buffer pH 6.5, 1 M sodium chloride containing 10% (v/v) ethanol. A Thermo Scientific ProPac Elite WCX 5 μm; 4 mm×250 mm column was used. Flow rate was set to 0.4 ml/min, detection was at a wavelength of 220 nm, the column running temperature was 40° C. (±0.8° C.) and the sample injection load was approx. 5.3 μg CNP-38 eq. (7 μL of a 0.75 mg/ml sample). The gradient is described in Table 23. Samples were diluted to 0.75 mg/ml with water.

TABLE 23

| Time [min] | % B |
|---|---|
| 0.0 | 2 |
| 2.0 | 2 |
| 17.0 | 16 |
| 17.1 | 98 |
| 25.0 | 98 |
| 25.1 | 2 |
| 43.0 | 2 |

RP-HPLC/MS to determine the content and purity of CNP (F33-45): Mobile phase A was composed of 0.05% aqueous TFA (v/v) and mobile phase B was composed of 0.04% TFA (v/v) in acetonitrile. A Waters Acquity CSH C18, 130 Å, 1.7 μm, 2.1×100 mm column was used. Flow rate was set to 0.3 ml/min, detection was at a wavelength of 215 nm, the column running temperature was 65° C. and the sample injection load was approx. 3 μg CNP-38 eq. (20 μL of a 0.135 mg/ml sample). Samples were diluted to 0.135 mg/mL with dilution buffer (10 mM succinic acid, 84 g/l trehalose dihydrate, adjusted to pH 5 with 1M Tris). The gradient is shown in Table 24. For LCMS the HPLC system was coupled to an LTQ Orbitrap Discovery XL instrument and ESI-MS was performed in the positive mode. The change in content of the TIM, T3M and T6M was determined by peak area comparison to the TO sample. For determination of the purity, all peaks higher than the LOQ (0.10%) and not present in the blank were integrated.

TABLE 24

| Time [min] | % B |
|---|---|
| 0.00 | 0.5 |
| 2.00 | 0.5 |
| 19.00 | 24.0 |
| 38.10 | 74.0 |
| 38.60 | 95.0 |
| 40.10 | 95.0 |
| 40.20 | 0.5 |
| 52.10 | 0.5 |

### Example 1: Synthesis of Compound (1)

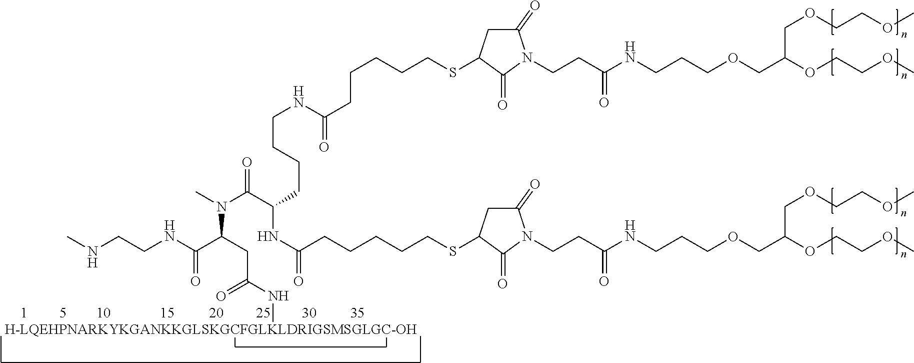

Compound (1) was synthesized as described in WO 2017/118693 A1 for conjugate 11i.

### Example 2: Stability of CNP-38 at Different pH Values in Succinate Buffer

The influence of the pH value of the liquid pharmaceutical formulation on the stability of CNP-38 was evaluated. For this purpose, six different formulations (F1, F2, F3, F4, F5, and F6) containing 2.8 mg/ml CNP-38 were prepared (Table 1).

TABLE 1

| | CNP-38 | Succinic acid | Trehalose dihydrate | | |
|---|---|---|---|---|---|
| | (mg/ml) | (mg/ml) | (mg/ml) | Base | pH |
| F1 | 2.8 | 1.18 | 84 | 1M Tris | 3.5 |
| F2 | 2.8 | 1.18 | 84 | 1M Tris | 4.0 |
| F3 | 2.8 | 1.18 | 84 | 1M Tris | 4.5 |
| F4 | 2.8 | 1.18 | 84 | 1M Tris | 5.0 |
| F5 | 2.8 | 1.18 | 84 | 1M Tris | 5.5 |
| F6 | 2.8 | 1.18 | 84 | 1M Tris | 6.0 |

The formulations were incubated at 37° C. for 7 days, after which they were assessed for overall purity and the presence of succinate adducts by using RP-HPLC and RP-HPLC-MS (MW of CNP-38+100 Da). The results indicate that following incubation the F3 formulation at pH 4.5 has the highest purity (shows the least degradation) and that succinate adducts were detected in F1 to F5 but not in F6 (see Table 2).

TABLE 2

| | | | Succinate adducts |
|---|---|---|---|
| Sample | pH | Purity [%] | detected |
| F1 | 3.5 | T0: 98.4 | Yes |
| | | T7 d: 93.3 | |
| | | Δ = 5.1 | |
| F2 | 4.0 | T0: 98.6 | Yes |
| | | T7 d: 93.9 | |
| | | Δ = 4.7 | |
| F3 | 4.5 | T0: 98.5 | Yes |
| | | T7 d: 94.0 | |
| | | Δ = 4.5 | |
| F4 | 5.0 | T0: 98.6 | Yes |
| | | T7 d: 93.6 | |
| | | Δ = 5.0 | |
| F5 | 5.5 | T0: 98.2 | Yes |
| | | T7 d: 91.6 | |
| | | Δ = 6.6 | |
| F6 | 6.0 | T0: 98.4 | No |
| | | T7 d: 88.6 | |
| | | Δ = 9.8 | |

### Example 3: Identification of Major CNP-38 Succinate Adduct

Incubated CNP-38 from Example 2 was used to isolate the major succinate isomer by RP-HPLC. In parallel, succinylated CNP-38 was generated de novo by incubating CNP-38 with succinic anhydride: To 10 mg CNP-38 in 5 ml PBS, 2.5 eq succinic anhydride in 0.6 ml PBS buffer were added, the pH of the mixture was adjusted by 4N NaOH to PH ˜6.5 and the mixture incubated for 30 min. The addition and pH adjustment were repeated three times, and the major isomer was isolated by RP-HPLC and lyophilized. By peptide mapping and LCMS analyses a succinylation was shown to be located at the N-terminus of the peptides for both compounds.

### Example 4: Stability of CNP-38 at Different pH Values in Acetate Buffer

The influence of the pH value of the liquid formulation on the stability of CNP-38 was evaluated. For this purpose, five different formulations containing CNP-38 (1.8 mg/ml) in acetate buffer (F7, F8, F9, F10 and F11) and one formulation in succinate buffer (F12) were prepared (Table 3).

TABLE 3

| | | | Trehalose | | |
|---|---|---|---|---|---|
| | Acetic acid | Succinic acid | dihydrate | | |
| | (mg/ml) | (mg/ml) | (mg/ml) | Base | pH |
| F7 | 0.60 | — | 84 | 0.1M NaOH | 3.5 |
| F8 | 0.60 | — | 84 | 0.1M NaOH | 4.0 |
| F9 | 0.60 | — | 84 | 0.1M NaOH | 4.5 |
| F10 | 0.60 | — | 84 | 0.1M NaOH | 5.0 |
| F11 | 0.60 | — | 84 | 0.1M NaOH | 5.5 |
| F12 | — | 1.18 | 84 | 1M Tris | 4.5 |

The formulations were incubated at 37° C. and samples were pulled for analysis after 8 days and 28 days. The samples were assessed for overall purity and the presence of acetate or succinate adducts by using RP-HPLC and RP-HPLC-MS. The results indicate that for the acetate formulations, the pH 4.0 (F8) and pH 4.5 (F9) formulations have the highest purities (least degradation) and that no acetate adducts were detected after RP-HPLC or RP-HPLC-MS evaluation in any of the five formulations. Succinate adducts were again detected in the formulation containing succinate (F12) and when comparing the equivalent acetate and succinate formulations (F9 vs F12 respectively) the acetate formulation gave higher purity after incubation (Table 4).

TABLE 4

| | | | Buffer adducts |
|---|---|---|---|
| Sample | pH | Purity [%] | detected |
| F7 | 3.5 | T0: 96.6 | No |
| | | T8 d: 93.0 (Δ = 3.6) | |
| | | T28 d: 84.2 (Δ = 12.4) | |
| F8 | 4.0 | T0: 96.7 | No |
| | | T8 d: 94.2 (Δ = 2.5) | |
| | | T28 d: 87.2 (Δ = 9.5) | |
| F9 | 4.5 | T0: 97.7 | No |
| | | T8 d: 94.5 (Δ: 3.2) | |
| | | T28 d: 87.4 (Δ = 10.3) | |
| F10 | 5.0 | T0: 95.6 | No |
| | | T8 d: 91.7 (Δ: 3.9) | |
| | | T28 d: 82.7 (Δ = 12.9) | |
| F11 | 5.5 | T0: 97.0 | No |
| | | T8 d: 90.4 (Δ = 6.6) | |
| | | T28 d: 76.5 (Δ = 20.5) | |
| F12 | 4.5 | T0: 96.6 | Yes |
| | | T8 d: 92.3 (Δ = 4.3) | |
| | | T28 d: 84.4 (Δ = 12.2) | |

### Example 5: Influence of the Base Used for pH Adjustment on the Stability of CNP-38 in a Liquid Formulation

The influence of the base used to adjust the pH of the liquid formulation on the stability of CNP-38 was evaluated. For this purpose, two different formulations containing CNP-38 (1.8 mg/ml) in acetate buffer (F13 and F14) were prepared. In F13 the pH was adjusted by using 1.0 M Tris solution and in F14 by using 0.1 M NaOH solution (Table 5). The samples were incubated at 37° C. for 29 days and then analyzed by RP-HPLC. The results showed only a minor difference in the purity between the two formulations, F13=84.9% and, F14=84.2%.

TABLE 5

| | | Anti- | Tonicity | Preser- | | |
|---|---|---|---|---|---|---|
| | Buffer | oxidant | Agent | vative | Base | pH |
| F13 | Acetic acid | — | Trehalose | — | 1.0M Tris | 4.5 |
| | 0.60 mg/ml | | dihydrate | | | |
| | | | 84 mg/ml | | | |
| F14 | Acetic acid | — | Trehalose | — | 0.1M NaOH | 4.5 |
| | 0.60 mg/ml | | dihydrate | | | |
| | | | 84 mg/ml | | | |

### Example 6: Influence of the Tonicity Agent on the Stability of CNP-38 in Liquid Formulation

The influence of the tonicity agent used in the liquid formulation on the stability of CNP-38 was evaluated. For this purpose, four formulations containing CNP-38 (1.8 mg/ml) in acetate buffer using four different tonicity agents were prepared (F14, F15, F16 and F17, Table 6). The samples were incubated at 37° C. for 29 days and then analyzed by RP-HPLC. The results showed only a minor difference in purity between the four formulations, F14=84.2%, F15=85.0%, F16=84.5% and F17=84.7%.

TABLE 6

| | | Anti- | Tonicity | Preser- | | |
|---|---|---|---|---|---|---|
| | Buffer | oxidant | Agent | vative | Base | pH |
| F14 | Acetic acid | — | Trehalose | — | 0.1M NaOH | 4.5 |
| | 0.60 mg/ml | | dihydrate | | | |
| | | | 84 mg/ml | | | |
| F15 | Acetic acid | — | Sodium | — | 0.1M NaOH | 4.5 |
| | 0.60 mg/ml | | Chloride | | | |
| | | | 8.4 mg/ml | | | |
| F16 | Acetic acid | — | Mannitol | — | 0.1M NaOH | 4.5 |
| | 0.60 mg/ml | | 52.1 mg/ml | | | |
| F17 | Acetic acid | — | Sucrose | — | 0.1M NaOH | 4.5 |
| | 0.60 mg/ml | | 97.9 mg/ml | | | |

### Example 7: Influence of the Antioxidant on the Stability of CNP-38 in a Liquid Formulation

The influence of the antioxidant used in the liquid formulation on the stability of CNP-38 was evaluated. For this purpose, four different formulations containing CNP-38 (1.8 mg/ml) were prepared (Table 7): one in acetate buffer without antioxidant (F14), one in acetate buffer containing 1.5 mg/ml methionine (F18), one in EDTA buffer (F19) and one in citrate buffer 10 (F20).

TABLE 7

| | | Anti- | Tonicity | Preser- | | |
|---|---|---|---|---|---|---|
| | Buffer | oxidant | Agent | vative | Base | pH |
| F14 | Acetic acid | — | Trehalose | — | 0.1M | 4.5 |
| | 0.60 mg/ml | | dihydrate | | NaOH | |
| | | | 84 mg/ml | | | |
| F18 | Acetic acid | Methionine | Trehalose | — | 0.1M | 4.5 |
| | 0.60 mg/ml | 1.5 mg/ml | dihydrate | | NaOH | |
| | | | 84 mg/ml | | | |
| F19 | EDTA | EDTA | Trehalose | — | 0.1M | 4.5 |
| | 2.9 mg/ml | | dihydrate | | NaOH | |
| | | | 84 mg/ml | | | |
| F20 | Citric acid | Citric acid | Trehalose | — | 0.1M | 4.5 |
| | 1.9 mg/ml | | dihydrate | | NaOH | |
| | | | 84 mg/ml | | | |

The samples were incubated at 37° C. for 29 days and then analyzed by RP-HPLC (Table 8). The results showed that the amount of methionine oxidation detected in the formulation containing methionine as antioxidant (F18 0.2%) was lower than in the formulation without antioxidant (F14, 0.5%). Moreover, the amount of methionine oxidation in the EDTA (F19, 1.6%) and citrate (F20, 0.7%) formulations was higher than in the formulation without antioxidant (F14). The results show that whilst adding methionine protects against oxidation of the methionine residue, neither EDTA nor citrate provides protection and possibly increases the amount of oxidation compared to an acetate buffer without antioxidant (Table 8). The purity of the two acetate formulations (with and without methionine) are essentially the same and the EDTA formulation is approximately 1% lower (83.2%). However, the citrate formulation has a significantly lower purity (79.0%) suggesting that in addition to not preventing oxidation it is also an unsuitable choice of buffer in comparison to acetate (Table 8).

TABLE 8

| | | Methionine Oxidation | Purity |
|---|---|---|---|
| Sample | Antioxidant | [%] | [%] |
| F14 | — | T0: 0.1 | T0: 96.4 |
| | | T29 d: 0.5 | T29 d: 84.2 |
| | | Δ = 0.4 | Δ = 12.2 |
| F18 | Methionine | T0: 0.1 | T0: 96.7 |
| | | T29 d: 0.2 | T29 d: 84.4 |
| | | Δ = 0.1 | Δ = 12.2 |
| F19 | EDTA | T0: 0.2 | T0: 96.2 |
| | | T29 d: 1.6 | T29 d: 83.2 |
| | | Δ = 1.4 | Δ = 13.0 |
| F20 | Citrate | T0: 0.1 | T0: 95.7 |
| | | T29 d: 0.7 | T29 d: 79.0 |
| | | Δ = 0.6 | Δ = 16.7 |

### Example 8: Influence of the Preservative on the Stability of CNP-38 in a Liquid Formulation

The influence of the preservative used in the liquid formulation on the stability of CNP-38 was evaluated. For this purpose, two different formulations containing CNP-38 (1.8 mg/ml) in acetate buffer with either 3 mg/ml m-cresol (F22) or 3 mg/ml phenol (F23) were prepared (Table 9). The samples were incubated at 37° C. for 29 days and then analyzed by RP-HPLC. The results showed only a minor difference in purity between the two formulations, F22=83.4% and F23=83.9% and the formulation without preservative F14=84.2%.

TABLE 9

| | | Anti- | Tonicity | Preser- | | |
|---|---|---|---|---|---|---|
| | Buffer | oxidant | Agent | vative | Base | pH |
| F22 | Acetic acid | — | Trehalose | m-Cresol | 0.1M NaOH | 4.5 |
| | 0.60 mg/ml | | dihydrate | 0.3% | | |
| | | | 84 mg/ml | | | |
| F23 | Acetic acid | — | Trehalose | Phenol | 0.1M NaOH | 4.5 |
| | 0.60 mg/ml | | dihydrate | 0.3% | | |
| | | | 84 mg/ml | | | |

### Example 9: Evaluation of the Effectiveness of m-Cresol as Preservative in a Liquid Formulation Comprising Compound (1)

The ability of m-cresol to preserve a liquid formulation containing compound (1) was evaluated. For this purpose, compound (1) was formulated at a concentration of 6.0 mg CNP-38 eq./ml in acetic acid with trehalose dihydrate and m-cresol at pH 5.0 (F24, Table 10).

TABLE 10

| | | Anti- | Tonicity | Preser- | | |
|---|---|---|---|---|---|---|
| | Buffer | oxidant | Agent | vative | Base | pH |
| F24 | Acetic acid | — | Trehalose | m-Cresol | 0.1M NaOH | 5.0 |
| | 0.60 mg/ml | | dihydrate | 0.285% | | |
| | | | 84 mg/ml | | | |

The formulation was tested for antimicrobial effectiveness against Staphylococcus aureus after 6 hours and 24 hours and against Candida albicans after 7 days and showed sufficient preservation (criteria A, Ph. Eur. 5.1.3.).

### Example 10: Stability Testing of Liquid Formulations Containing Compound (1)

The influence of the pH, the antioxidant, the tonicity agent and the CNP-38 content of a liquid formulation on the stability of compound (1) was evaluated. For this purpose, eight formulations were prepared in acetate buffer using m-cresol as preservative (Table 11): one each at pH 4.0 (F25), pH 4.5 (F26) and pH 5.0 (F27) using trehalose dihydrate as tonicity agent and a concentration of 3.6 mg CNP-38 eq./ml and the same using mannitol as tonicity agent (F28, F29 and F30), and additionally, a 3.6 mg CNP-38 eq./ml formulation using trehalose dihydrate and containing methionine as antioxidant (F31) and a trehalose dihydrate formulation containing 6.0 mg CNP-38 eq./ml were prepared (F32, Table 11). All the formulations were incubated at 5° C. and one trehalose dihydrate comprising formulation (F26) and one mannitol comprising formulation (F29) were also incubated at 25° C.

TABLE 11

| | CNP-38 | | | Tonicity | | | |
|---|---|---|---|---|---|---|---|
| | eq Content | Buffer | Antioxidant | Agent | Preservative | Base | pH |
| F25 | 3.6 mg/ml | Acetic acid | — | Trehalose | m-Cresol | NaOH | 4.0 |
| | | 0.60 mg/ml | | dihydrate | 0.3% | | |
| | | | | 84 mg/ml | | | |
| F26 | 3.6 mg/ml | Acetic acid | — | Trehalose | m-Cresol | NaOH | 4.5 |
| | | 0.60 mg/ml | | dihydrate | 0.3% | | |
| | | | | 84 mg/ml | | | |
| F27 | 3.6 mg/ml | Acetic acid | — | Trehalose | m-Cresol | NaOH | 5.0 |
| | | 0.60 mg/ml | | dihydrate | 0.3% | | |
| | | | | 84 mg/ml | | | |
| F28 | 3.6 mg/ml | Acetic acid | — | Mannitol | m-Cresol | NaOH | 4.0 |
| | | 0.60 mg/ml | | 41 mg/ml | 0.3% | | |
| F29 | 3.6 mg/ml | Acetic acid | — | Mannitol | m-Cresol | NaOH | 4.5 |
| | | 0.60 mg/ml | | 41 mg/ml | 0.3% | | |
| F30 | 3.6 mg/ml | Acetic acid | — | Mannitol | m-Cresol | NaOH | 5.0 |
| | | 0.60 mg/ml | | 41 mg/ml | 0.3% | | |
| F31 | 3.6 mg/ml | Acetic acid | Methionine | Trehalose | m-Cresol | NaOH | 4.5 |
| | | 0.60 mg/ml | 1.5 mg/ml | dihydrate | 0.3% | | |
| | | | | 84 mg/ml | | | |
| F32 | 6.0 mg/ml | Acetic acid | — | Trehalose | m-Cresol | NaOH | 4.5 |
| | | 0.60 mg/ml | | dihydrate | 0.3% | | |
| | | | | 84 mg/ml | | | |

The trehalose dihydrate containing formulations were analyzed after 3, 6, and 24 months by SE-HPLC and RP-HPLC. No loss of content or purity was detected after 6 months for any of the formulations incubated at 5° C., except for F27 (pH 5.0 formulation), which contained 0.12% free CNP-38 after 6 months incubation indicating that higher pH values lead to higher amounts of free CNP-38 (Table 12). After 24 months the purity remained high for all of the formulations at 5° C., and small amounts of free CNP were observed for all formulations at pH 4.5 and above (F26, F31. F32), indicating that higher pH values lead to higher amounts of free CNP-38. No loss of content was observed.

TABLE 12

| | Purity by SE-HPLC (%) | | | | Purity by RP-HPLC (%) | | | | Free CNP by RP-HPLC (%) | | | |
|---|---|---|---|---|---|---|---|---|---|---|---|---|
| | T0 | 3 M | 6 M | 24 M | T0 | 3 M | 6 M | 24 M | T0 | 3 M | 6 M | 24 M |
| F25 | 100 | 100 | 100 | 99.7 | 99.6 | 99.6 | 99.6 | 99.7 | <LOQ | <LOQ | <LOQ | <LOQ |
| 5° C. | | | | | | | | | | | | |
| F26 | 100 | 100 | 100 | 99.7 | 99.6 | 99.6 | 99.6 | 99.5 | <LOQ | <LOQ | <LOQ | 0.22 |
| 5° C. | | | | | | | | | | | | |
| F27 | 100 | 100 | 100 | 99.7 | 99.6 | 99.7 | 99.5 | 98.9 | <LOQ | <LOQ | 0.12 | 0.48 |
| 5° C. | | | | | | | | | | | | |
| F31 | 100 | 100 | 100 | 99.7 | 99.7 | 99.7 | 99.6 | 99.5 | <LOQ | <LOQ | <LOQ | 0.20 |
| 5° C. | | | | | | | | | | | | |
| F32 | 100 | 100 | 100 | 99.7 | 99.6 | 99.7 | 99.6 | 99.4 | <LOQ | <LOQ | <LOQ | 0.18 |
| 5° C. | | | | | | | | | | | | |
| F26 | 100 | 99.3 | 98.9 | 95.2 | 99.7 | 98.3 | 96.7 | 86.6 | <LOQ | 0.72 | 1.3 | 2.9 |
| 25° C. | | | | | | | | | | | | |

LOQ (RP-HPLC) = 0.10%

The trehalose dihydrate containing formulations were analyzed after 3, 6, and 24 months for methionine oxidation and isoaspartate by peptide mapping. For all formulations incubated at 5° C. both methionine oxidation (Met[O]) and isoaspartate remained below the LOQ for 6 months. After 24 months methionine oxidation slightly above LOQ was observed for all formulations with the exception of the methionine containing formulation F31. Approximately 2% of isoaspartate formation was observed for all formulations after 24 months. Incubation of F26 at 25° C. led to an increased amount of methionine oxidation and larger amounts of isoaspartate (Table 13).

TABLE 13

| | Met[O] by Peptide Mapping (%) | | | | Isoaspartate Formation by Peptide Mapping (%) | | | |
|---|---|---|---|---|---|---|---|---|
| | T0 | 3 M | 6 M | 24 M | T0 | 3 M | 6 M | 24 M |
| F25 | <LOQ | <LOQ | <LOQ | 1.1 | <LOQ | <LOQ | <LOQ | 2.4 |
| 5° C. | | | | | | | | |
| F26 | <LOQ | <LOQ | <LOQ | 1.2 | <LOQ | <LOQ | <LOQ | 1.9 |
| 5° C. | | | | | | | | |
| F27 | <LOQ | <LOQ | <LOQ | 1.2 | <LOQ | <LOQ | <LOQ | 1.5 |
| 5° C. | | | | | | | | |
| F31 | <LOQ | <LOQ | <LOQ | <LOQ | <LOQ | <LOQ | <LOQ | 1.7 |
| 5° C. | | | | | | | | |
| F32 | <LOQ | <LOQ | <LOQ | 1.0 | <LOQ | <LOQ | <LOQ | 2.1 |
| 5° C. | | | | | | | | |
| F26 | <LOQ | 1.3 | 1.5 | 3.2 | <LOQ | 2.6 | 7.2 | 23.3 |
| 25° C. | | | | | | | | |

LOQ = 1.0%

The three mannitol containing formulations, F28 (pH 4.0), F29 (pH 4.5), and F30 (pH 5.0) were analyzed after 3 months, 6 months, and 12 months by SE-HPLC and RP-HPLC. In SE-HPLC a loss of purity of 0.5% was observed for the 5° C. incubated samples. In RP-HPLC the pH 4.0 and pH 4.5 samples remain unchanged with regards to content and purity, whilst the purity of the pH 5.0 sample is slightly reduced after 12 months at 5° C. After 12 months at 5° C. there was 0.23% free CNP-38 in F30, 0.10% in F29 and below LOQ in F28 indicating that higher pH values lead to higher amounts of free CNP-38 (Table 14).

TABLE 14

| | Purity by SE-HPLC (%) | | | | Purity by RP-HPLC (%) | | | | Free CNP by RP-HPLC (%) | | | |
|---|---|---|---|---|---|---|---|---|---|---|---|---|
| | T0 | 3 M | 6 M | 12 M | T0 | 3 M | 6 M | 12 M | T0 | 3 M | 6 M | 12 M |
| F28 | 100 | 100 | 100 | 99.5 | 99.6 | 99.6 | 99.6 | 99.6 | <LOQ | <LOQ | <LOQ | <LOQ |
| 5° C. | | | | | | | | | | | | |
| F29 | 100 | 100 | 100 | 99.5 | 99.6 | 99.6 | 99.6 | 99.7 | <LOQ | <LOQ | <LOQ | <LOQ |
| 5° C. | | | | | | | | | | | | |
| F30 | 100 | 100 | 100 | 99.5 | 99.6 | 99.7 | 99.5 | 99.3 | <LOQ | <LOQ | 0.11 | 0.23 |
| 5° C. | | | | | | | | | | | | |
| F29 | 100 | 99.3 | 98.9 | 97.8 | 99.6 | 98.2 | 96.8 | 93.8 | <LOQ | 0.68 | 1.2 | 2.0 |
| 25° C. | | | | | | | | | | | | |

LOQ = 0.1% (RP-HPLC)

The mannitol containing formulations were analyzed after 3 months, 6 months, and 12 months for methionine oxidation and isoaspartate by peptide mapping. For all formulations incubated at 5° C. the amount of methionine oxidation remained below the LOQ (1.0%) and isoaspartate was only detected in F28 (1.3%) and F29 (1.1%) after 12 months indicating higher amounts of isoaspartate at lower pH values. Incubation of F29 at 25° C. led to an increasing amount of methionine oxidation and isoaspartate over time (Table 15).

TABLE 15

| | Met[O] by Peptide Mapping (%) | | | | Isoaspartate formation by Peptide Mapping (%) | | | |
|---|---|---|---|---|---|---|---|---|
| | T0 | 3 M | 6 M | 12 M | T0 | 3 M | 6 M | 12 M |
| F28 | <LOQ | <LOQ | <LOQ | <LOQ | <LOQ | <LOQ | <LOQ | 1.3 |
| 5° C. | | | | | | | | |
| F29 | <LOQ | <LOQ | <LOQ | <LOQ | <LOQ | <LOQ | <LOQ | 1.1 |
| 5° C. | | | | | | | | |
| F30 | <LOQ | <LOQ | <LOQ | <LOQ | <LOQ | <LOQ | <LOQ | <LOQ |
| 5° C. | | | | | | | | |
| F29 | <LOQ | 1.2 | 1.4 | 2.1 | <LOQ | 2.7 | 7.1 | 13.6 |
| 25° C. | | | | | | | | |

LOQ = 1.0%

The SE-HPLC, RP-HPLC, and peptide mapping results for the trehalose dihydrate formulations (F25, F26 and F27) and the mannitol formulations (F28, F29 and F30) incubated at 5° C. are similar and indicate that trehalose dihydrate and mannitol perform equally well. This is supported by comparing the results of trehalose dihydrate formulation F26 and mannitol formulation F29 incubated at 25° C., which are also similar.

### Example 11: Stability Testing of Liquid Formulations Containing CNP

The influence of the buffering agent, pH, tonicity agent, detergent, and preservative on the stability of CNP-38 was evaluated. For this purpose, nine formulations of CNP-38 were prepared at a concentration of 2 mg/mL and incubated for 6 months (Table 16). One formulation each at pH 4.5 (F33), pH 5.0 (F34) and pH 4.0 (F35) in acetate buffer were prepared. L-methionine was used to prevent the oxidation of the methionine residue in the peptide, trehalose dihydrate was used as a tonicity or isotonicity agent, Poloxamer 188 as a detergent and m-cresol as a preservative. Another formulation at pH 4.5 containing the same excipients was prepared, in which trehalose was switched to mannitol (F37). Formulation F38 was the same as formulation F33 but did not contain Poloxamer 188. The formulations were incubated at 5° C. To evaluate whether acetate adducts are formed in acetate buffer, the same formulation as in F33 was incubated at accelerated conditions at 25° C. (F33, 25° C.). For comparison, a similar formulation using succinic acid instead of acetic acid was prepared to check for succinylation of the peptide at 25° C. (F36). A citrate formulation containing methionine, trehalose, mannitol, and Polysorbate-80 (PS80) as detergent was prepared (F39). A preserved version of this formulation was also prepared by adding m-cresol and exchanging PS80 with Poloxamer 188, due to the incompatibility of PS80 and m-cresol (F40). The formulations were incubated at 5° C. for 6 months and at 25° C. for 1 month. Formulations of CNP-22, CNP-27, CNP-53 and PG-CNP-37 with the same excipients as for F33 were prepared and incubated at 5° C. (F41, F43, F44, F45). In order to investigate the succinylation of CNP-22, a formulation in succinate buffer was prepared and incubated at 25° C. (F42).

TABLE 16

| | | | | | L- | Tonicity | | | | |
|---|---|---|---|---|---|---|---|---|---|---|
| | | | | Buffer | Met | agent | | Preservative | Base | |
| | Peptide | Temp | | (mM) | (mM) | (g/l) | Detergent | (%) | (1M) | pH |
| F33 | CNP-38 | 5° | C. | 10 | 10 | 84 | 0.1% | 0.3 | NaOH | 4.5 |
| | | | | Acetate | | Trehalose | Poloxamer | m-Cresol | | |
| | | | | | | dihydrate | 188 | | | |
| F34 | CNP-38 | 5° | C. | 10 | 10 | 84 | 0.1% | 0.3 | NaOH | 5.0 |
| | | | | Acetate | | Trehalose | Poloxamer | m-Cresol | | |
| | | | | | | dihydrate | 188 | | | |
| F35 | CNP-38 | 5° | C. | 10 | 10 | 84 | 0.1% | 0.3 | NaOH | 4.0 |
| | | | | Acetate | | Trehalose | Poloxamer | m-Cresol | | |
| | | | | | | dihydrate | 188 | | | |
| F33 | CNP-38 | 25° | C. | 10 | 10 | 84 | 0.1% | 0.3 | NaOH | 4.5 |
| | | | | Acetate | | Trehalose | Poloxamer | m-Cresol | | |
| | | | | | | dihydrate | 188 | | | |
| F36 | CNP-38 | 25° | C. | 10 | 10 | 84 | 0.1% | 0.3 | NaOH | 4.5 |
| | | | | Succinate | | Trehalose | Poloxamer | m-Cresol | | |
| | | | | | | dihydrate | 188 | | | |
| F37 | CNP-38 | 5° | C. | 10 | 10 | 41 | 0.1% | 0.3 | NaOH | 4.5 |
| | | | | Acetate | | Mannitol | Poloxamer | m-Cresol | | |
| | | | | | | | 188 | | | |
| F38 | CNP-38 | 5° | C. | 10 | 10 | 84 | — | 0.3 | NaOH | 4.5 |
| | | | | Acetate | | Trehalose | | m-Cresol | | |
| | | | | | | dihydrate | | | | |
| F39 | CNP-38 | 5° | C. | 5 Citrate | 4.89 | 58.01 | 0.05 | — | NaOH | 5.5 |
| | | | | | | Trehalose | mg/mL | | | |
| | | | | | | dihydrate, | PS80 | | | |
| | | | | | | 15 | | | | |
| | | | | | | mannitol | | | | |
| F40 | CNP-38 | 5° | C. | 5 Citrate | 4.89 | 58.01 | 0.1% | 0.3 | NaOH | 5.5 |
| | | | | | | Trehalose | Poloxamer | m-Cresol | | |
| | | | | | | dihydrate, | 188 | | | |
| | | | | | | 15 | | | | |
| | | | | | | mannitol | | | | |
| F39 | CNP-38 | 25° | C. | 5 Citrate | 4.89 | 58.01 | 0.05 | — | NaOH | 5.5 |
| | | | | | | Trehalose | mg/mL | | | |
| | | | | | | dihydrate, | PS80 | | | |
| | | | | | | 15 | | | | |
| | | | | | | mannitol | | | | |
| F40 | CNP-38 | 25° | C. | 5 Citrate | 4.89 | 58.01 | 0.1% | 0.3 | NaOH | 5.5 |
| | | | | | | Trehalose | Poloxamer | m-Cresol | | |
| | | | | | | dihydrate, | 188 | | | |
| | | | | | | 15 | | | | |
| | | | | | | mannitol | | | | |
| F41 | CNP-22 | 5° | C. | 10 | 10 | 84 | 0.1% | 0.3 | NaOH | 4.5 |
| | | | | Acetate | | Trehalose | Poloxamer | m-Cresol | | |
| | | | | | | dihydrate | 188 | | | |
| F42 | CNP-22 | 25° | C. | 10 | 10 | 84 | 0.1% | 0.3 | NaOH | 4.5 |
| | | | | Succinate | | Trehalose | Poloxamer | m-Cresol | | |
| | | | | | | dihydrate | 188 | | | |
| F43 | CNP-27 | 5° | C. | 10 | 10 | 84 | 0.1% | 0.3 | NaOH | 4.5 |
| | | | | Acetate | | Trehalose | Poloxamer | m-Cresol | | |
| | | | | | | dihydrate | 188 | | | |
| F44 | CNP-53 | 5° | C. | 10 | 10 | 84 | 0.1% | 0.3 | NaOH | 4.5 |
| | | | | Acetate | | Trehalose | Poloxamer | m-Cresol | | |
| | | | | | | dihydrate | 188 | | | |
| F45 | PG- | 5° | C. | 10 | 10 | 84 | 0.1% | 0.3 | NaOH | 4.5 |
| | CNP-37 | | | Acetate | | Trehalose | Poloxamer | m-Cresol | | |
| | | | | | | dihydrate | 188 | | | |

The results from the RP-HPLC/MS analyses are shown in Table 17.

TABLE 17

| | | | | | | Succinate/ |
|---|---|---|---|---|---|---|
| | | Asp(28) | | Deamid | | acetate/ |
| | | trun- | | & | Aspar- | citrate |
| | CNP | cation | Met[O] | Iso Asp | timide(28) | adducts |
| Sample | (%) | (%) | (%) | (%) | (%) | (%) |
| F33 | T0: 98.0 | <LOQ | <LOQ | 1.4 | 0.1 | —* |
| | T1M: 97.9 | <LOQ | 0.1 | 1.5 | 0.2 | — |
| | Δ = 0.1 | | | | | |
| | T3M: 97.5 | <LOQ | 0.1 | 1.7 | 0.6 | — |
| | Δ = 0.5 | | | | | |
| | T6M: 96.9 | <LOQ | 0.2 | 1.7 | 0.9 | — |
| | Δ = 1.1 | | | | | |
| F34 | T0: 98.2 | <LOQ | <LOQ | 1.5 | 0.1 | — |
| | T1M: 98.2 | <LOQ | <LOQ | 1.4 | 0.2 | — |
| | Δ = 0 | | | | | |
| | T3M: 97.4 | <LOQ | 0.1 | 1.8 | 0.4 | — |
| | Δ = 0.6 | | | | | |
| | T6M: 97.1 | <LOQ | 0.1 | 2.1 | 0.6 | — |
| | Δ = 1.1 | | | | | |
| F35 | T0: 97.7 | <LOQ | <LOQ | 1.3 | 0.1 | — |
| | T1M: 97.7 | <LOQ | <LOQ | 1.3 | 0.3 | — |
| | Δ = 0 | | | | | |
| | T3M: 97.0 | 0.1 | 0.1 | 1.4 | 0.7 | — |
| | Δ = 0.7 | | | | | |
| | T6M: 96.3 | 0.2 | 0.1 | 1.5 | 1.3 | — |
| | Δ = 1.4 | | | | | |
| F33 | T0: 97.9 | <LOQ | <LOQ | 1.4 | 0.1 | — |
| 25° C. | T1M: 95.0 | <LOQ | 0.1 | 3.0 | 1.5 | — |
| | Δ = 2.9 | | | | | |
| | T3M: 89.5 | 0.3 | 0.1 | 7.1 | 2.4 | — |
| | Δ = 8.4 | | | | | |
| | T6M: 82.8 | 0.5 | 0.2 | 10.9 | 2.6 | — |
| | Δ = 15.1 | | | | | |
| F36 | T0: 98.1 | <LOQ | <LOQ | 1.2 | 0.2 | <LOQ |
| | T1M: 94.5 | 0.2 | 0.1 | 2.7 | 1.8 | 0.2 |
| | Δ = 3.6 | | | | | |
| | T3M: 87.7 | 0.6 | 0.2 | 6.2 | 2.4 | 0.8 |
| | Δ = 10.4 | | | | | |
| | T6M: 80.3 | 1.0 | 0.2 | 10.9 | 3.2 | 1.5 |
| | Δ = 17.8 | | | | | |
| F37 | T0: 98.0 | <LOQ | <LOQ | 1.3 | 0.1 | — |
| | T1M: 97.6 | <LOQ | <LOQ | 1.4 | 0.3 | |
| | Δ = 0.4 | | | | | |
| | T3M: 97.5 | <LOQ | 0.1 | 1.5 | 0.5 | — |
| | Δ = 0.5 | | | | | |
| | T6M: 97.1 | <LOQ | 0.1 | 1.7 | 0.9 | — |
| | Δ = 0.9 | | | | | |
| F38 | T0: 98.0 | <LOQ | <LOQ | 1.4 | 0.1 | — |
| | T1M: 98.0 | <LOQ | 0.1 | 1.4 | 0.2 | — |
| | Δ = 0 | | | | | |
| | T3M: 97.6 | <LOQ | 0.1 | 1.5 | 0.6 | — |
| | Δ = 0.4 | | | | | |
| | T6M: 97.2 | <LOQ | 0.1 | 1.6 | 0.9 | — |
| | Δ = 0.8 | | | | | |
| F39 | T0: 98.2 | <LOQ | <LOQ | 1.4 | 0.1 | — |
| | T1M: 97.6 | <LOQ | 0.1 | 1.6 | 0.2 | — |
| | Δ = 0.6 | | | | | |
| | T3M: 97.3 | <LOQ | 0.1 | 1.8 | 0.3 | <LOQ |
| | Δ = 0.9 | | | | | |
| | T6M: 96.5 | <LOQ | 0.2 | 1.9 | 0.5 | — |
| | Δ = 1.7 | | | | | |
| F40 | T0: 98.0 | <LOQ | 0.1 | 1.4 | 0.1 | — |
| | T1M: 97.7 | <LOQ | 0.1 | 1.6 | 0.2 | — |
| | Δ = 0.3 | | | | | |
| | T3M: 97.0 | <LOQ | 0.2 | 2.1 | 0.4 | <LOQ |
| | Δ = 1.0 | | | | | |
| | T6M: 96.5 | <LOQ | 0.2 | 2.1 | 0.5 | — |
| | Δ = 1.5 | | | | | |
| F39 | T1M: 71.4 | 0.2 | 0.2 | 23.8 | 0.5 | 0.7 |
| 25° C. | | | | | | |
| F40 | T1M: 70.3 | 0.2 | 0.2 | 24.8 | 0.5 | 0.7 |
| 25° C. | | | | | | |
| F41 | T0: 91.4 | <LOQ | 0.1 | 5.8 | <LOQ | — |
| | T1M: 90.8 | <LOQ | 0.2 | 5.8 | 0.2 | — |
| | Δ = 0.6 | | | | | |
| | T3M: 90.6 | <LOQ | 0.2 | 6.2 | 0.6 | — |
| | Δ = 0.8 | | | | | |
| | T6M: 90.1 | <LOQ | 0.2 | 6.2 | 1.0 | — |
| | Δ = 1.3 | | | | | |
| F42 | T0: 91.2 | <LOQ | 0.1 | 5.8 | <LOQ | — |
| | T1M: 88.2 | 0.2 | 0.2 | 5.9 | 2.0 | 0.3 |
| | Δ = 3.0 | | | | | |
| | T3M: 83.3 | 0.5 | 0.3 | 7.8 | 4.1 | 0.8 |
| | Δ = 7.9 | | | | | |
| | T6M: 78.4 | 0.9 | 0.3 | 11.3 | 4.2 | 1.2 |
| | Δ = 12.8 | | | | | |
| F43 | T0: 94.6 | <LOQ | 0.3 | 1.7 | <LOQ | — |
| | T1M: 94.5 | 0.1 | 0.4 | 1.9 | 0.2 | — |
| | Δ = 0.1 | | | | | |
| | T3M: 94.0 | 0.1 | 0.4 | 1.9 | 0.5 | — |
| | Δ = 0.6 | | | | | |
| | T6M: 93.4 | 0.2 | 0.5 | 2.0 | 0.9 | — |
| | Δ = 1.2 | | | | | |
| F44 | T0: 81.2 | <LOQ | 3.2 | 6.0 | <LOQ | — |
| | T1M: 80.8 | <LOQ | 3.3 | 6.5 | 0.2 | — |
| | Δ = 0.4 | | | | | |
| | T3M: 80.7 | <LOQ | 3.3 | 7.1 | 0.4 | — |
| | Δ = 0.5 | | | | | |
| | T6M: 79.4 | <LOQ | 3.3 | 7.7 | 0.6 | — |
| | Δ = 1.8 | | | | | |
| F45 | T0: 86.7 | <LOQ | 0.2 | 6.5 | 0.1 | — |
| | T1M: 86.7 | <LOQ | 0.3 | 7.1 | 0.2 | — |
| | Δ = 0 | | | | | |
| | T3M: 86.5 | <LOQ | 0.3 | 7.2 | 0.4 | — |
| | Δ = 0.2 | | | | | |
| | T6M: 85.6 | 0.1 | 0.3 | 7.5 | 0.7 | — |
| | Δ = 1.1 | | | | | |

*“—” means that the impurity was not detected.

Overall high purities are observed for the peptides when incubated in formulation buffer at 5° C. Increased amounts of deamidation are observed at pH 5.0 (F34) as compared to pH 4.5 (F33) and pH 4.0 (F35). On the other hand, more aspartimide (28) and more Asp (28) truncation can be observed at pH 4.0 (F35) than at pH 4.5 (F33) and pH 5.0 (F34). Replacing the tonicity agent trehalose dihydrate (F33) with mannitol (F37) did not have a significant influence on the amounts of formed impurities. Very similar results have also been observed when leaving out the detergent Poloxamer 188 (F39). The purity results and amounts of formed impurities are similar to F33. None of the mentioned formulations F33, F34, F35, F37, or F38 revealed formation of acetylated species. The two citrate formulations incubated at 5° C. (F39 and F40) showed increased amounts of deamidation in comparison to F33 and less aspartimide (28) formation. Both observations are considered to be related to the increased pH of 5.5 in F39 and F40 compared to pH 4.5 in F33. Small amounts of citrate adducts were observed by LC-MS after 3 months incubation at 5° C. After 1 month of incubation of F39 and F40 at 25° C., citrate adducts were observed in both samples. The formation of acetylated or succinylated species was checked at accelerated conditions at 25° C. in either acetate buffer (F33, 25° C.) or succinate buffer (F36). While no acetylated species were observed by LC-MS, several succinylated species were detected. Additionally, increased amounts of Asp (28) truncation and aspartimide (28) were detected in the succinate formulation (F36). The purity of CNP-38 in the acetate formulation which was incubated at 25° C. (F33, 25° C.) was significantly lower than the purity of the same formulation incubated at 5° C. (F33), as more impurities had formed due to accelerated conditions. CNP-22, CNP-27, CNP-53 and PG-CNP-37 (F41, F43, F45, F46) showed similar levels of impurity formation after incubation in acetate buffer at pH 4.5 for 6 months at 5° C. No significant differences in terms of the stability of these peptides were observed under the conditions displayed in Table 16. No acetylated peptides were detected. CNP-22 in succinate buffer at 25° C. (F42) showed comparable amounts of succinylation as for CNP-38 stored at the same conditions (F36). The peptide content remained stable. Additionally, only a slight increase in methionine oxidation was observed.

### Example 12: Stability Testing of Liquid Formulations Containing Compound (1)

The influence of the buffering agent on the stability of compound (1) was evaluated. For this purpose, six formulations of CNP-38 were prepared at a nominal concentration of 2.2 mg/ml and incubated for 6 months (Table 18). One formulation was prepared in acetate buffer, containing trehalose, methionine, m-cresol and Poloxamer 188 (F46) at pH 4.5. A similar formulation containing phenol instead of m-cresol as a preservative was also prepared (F48). Both formulations were incubated at 5° C. To check for the formation of acetate adducts, F46 was incubated at 25° C. (F46, 25° C.). For comparison, a formulation in succinate buffer was also prepared and incubated at 25° C. (F47). Two formulations of compound (1) in citrate were prepared. One of the formulations contained PS80 as a detergent and no preservative (F49), while the other one contained Poloxamer 188 and the preservative m-cresol (F50). The formulations were incubated at 5° C.

TABLE 18

| | | | | L- | Tonicity | | | | |
|---|---|---|---|---|---|---|---|---|---|
| | | T | Buffer | Met | agent | | Preservative | Base | |
| | Compound | (° C.) | (mM) | (mM) | (g/l) | Detergent | (%) | (1M) | pH |
| F46 | Compound | 5 | 10 | 10 | 84 | 0.1% | 0.3 | NaOH | 4.5 |
| | (1) | | Acetate | | Trehalose | Poloxamer | m-Cresol | | |
| | | | | | dihydrate | 188 | | | |
| F46 | Compound | 25 | 10 | 10 | 84 | 0.1% | 0.3 | NaOH | 4.5 |
| | (1) | | Acetate | | Trehalose | Poloxamer | m-Cresol | | |
| | | | | | dihydrate | 188 | | | |
| F47 | Compound | 25 | 10 | 10 | 84 | 0.1% | 0.3 | NaOH | 4.5 |
| | (1) | | Succinate | | Trehalose | Poloxamer | m-Cresol | | |
| | | | | | dihydrate | 188 | | | |
| F48 | Compound | 5 | 10 | 10 | 84 | 0.1% | 0.5 | NaOH | 4.5 |
| | (1) | | Acetate | | Trehalose | Poloxamer | m-Phenol | | |
| | | | | | dihydrate | 188 | | | |
| F49 | Compound | 5 | 5 Citrate | 4.89 | 58.01 | 0.05 | — | NaOH | 5.5 |
| | (1) | | | | Trehalose | mg/ml | | | |
| | | | | | dihydrate, | PS80 | | | |
| | | | | | 15 | | | | |
| | | | | | mannitol | | | | |
| F50 | Compound | 5 | 5 Citrate | 4.89 | 58.01 | 0.1% | 0.3 | NaOH | 5.5 |
| | (1) | | | | Trehalose | Poloxamer | m-Cresol | | |
| | | | | | dihydrate, | 188 | | | |
| | | | | | 15 | | | | |
| | | | | | mannitol | | | | |

The results from RP-HPLC and SEC analyses are shown in Table 19.

TABLE 19

| | | | Free CNP | Content |
|---|---|---|---|---|
| | Purity by | Purity by | by RP- | by RP-HPLC |
| Sample | SE-HPLC (%) | RP-HPLC (%) | HPLC (%) | (mg CNP-38/ml) |
| F46 | T0: 100.0 | 99.6 | <LOQ | 2.4 |
| | T1 M: 100.0 | 99.6 | <LOQ | 2.3 |
| | Δ = 0 | Δ = 0 | | |
| | T3 M: 100.0 | 99.6 | <LOQ | 2.3 |
| | Δ = 0 | Δ = 0 | | |
| | T6 M: 100.0 | 99.6 | <LOQ | 2.3 |
| | Δ = 0 | Δ = 0 | | |
| F46 | T0: 100.0 | 99.6 | <LOQ | 2.4 |
| (25° C.) | T1 M: 99.5 | 99.0 | 0.3 | 2.4 |
| | Δ = 0.5 | Δ = 0.6 | | |
| | T3 M: 99.2 | 97.8 | 0.9 | 2.3 |
| | Δ = 0.8 | Δ = 1.8 | | |
| | T6 M: 98.8 | 96.3 | 1.5 | 2.3 |
| | Δ = 1.2 | Δ = 3.3 | | |
| F47 | T0: 99.8 | 99.6 | <LOQ | 2.4 |
| | T1 M: 99.5 | 99.1 | 0.2 | 2.4 |
| | Δ = 0.3 | Δ = 0.5 | | |
| | T3 M: 99.3 | 97.9 | 0.7 | 2.4 |
| | Δ = 0.5 | Δ = 1.7 | | |
| | T6 M: 98.9 | 96.6 | 1.2 | 2.3 |
| | Δ = 0.9 | Δ = 3.0 | | |
| F48 | T0: 99.8 | 99.6 | <LOQ | 2.3 |
| | T1 M: 100.0 | 99.6 | <LOQ | 2.3 |
| | Δ = 0.2 | Δ = 0 | | |
| | T3 M: 100.0 | 99.6 | <LOQ | 2.3 |
| | Δ = 0.2 | Δ = 0 | | |
| | T6 M: 99.8 | 99.6 | <LOQ | 2.3 |
| | Δ = 0 | Δ = 0 | | |
| F49 | T0: 99.8 | 99.6 | <LOQ | 2.4 |
| | T1 M: 99.6 | 99.6 | <LOQ | 2.4 |
| | Δ = 0.2 | Δ = 0 | | |
| | T3 M: 99.5 | 99.4 | 0.1 | 2.4 |
| | Δ = 0.3 | Δ = 0.2 | | |
| | T6 M: 99.5 | 99.1 | 0.2 | 2.4 |
| | Δ = 0.3 | Δ = 0.5 | | |
| F50 | T0: 99.8 | 99.6 | <LOQ | 2.3 |
| | T1 M: 99.9 | 99.6 | <LOQ | 2.3 |
| | Δ = 0.1 | Δ = 0 | | |
| | T3 M: 99.8 | 99.4 | 0.2 | 2.3 |
| | Δ = 0 | Δ = 0.2 | | |
| | T6 M: 99.5 | 99.1 | 0.3 | 2.3 |
| | Δ = 0.3 | Δ = 0.5 | | |

LOQ (RP-HPLC) = 0.10%

No changes in the purity of compound (1) were observed in either SE-HPLC or RP-HPLC for the formulation in acetate buffer at pH 4.5 with m-cresol (F46) for 6 months at 5° C. The amount of free CNP-38 was below LOQ at all timepoints, and the content of compound (1) remained stable. The same results were obtained for formulation F48, which contained phenol instead of m-cresol as preservative. A slight decrease in the purity of compound (1) was observed via both methods for the citrate formulations, of which F49 did not contain a preservative and F50 did. Additionally, values above LOQ for free CNP-38 were obtained, likely due to the higher pH of 5.5. The content of compound (1) remained stable throughout the 6 months. A significant decrease in purity was observed via both analytical methods for the formulations in acetate (F46) and succinate (F47) buffer, under accelerated conditions at 25° C. Values of free CNP-38 above 1% were attributed to the higher temperature. The content of compound (1) was not affected by the incubation. Additional impurities not present in the acetate formulation (F46) were detected by IEX analysis in the succinate formulation (F47)(Table 20).

TABLE 20

Additional impurities detected by IEX (%)

| Sample | T0 | T1 M | T3 M | T6 M |
|---|---|---|---|---|
| F47 | — | 0.23 | 1.4 | 2.5 |

### Abbreviations

CNP—C-type natriuretic peptide
EDTA—ethylenediaminetetraacetic acid
ESI-MS—electrospray ionization mass spectrometry
HPLC—high-performance liquid chromatography
IEX-HPLC—ion exchange high-performance liquid chromatography
IGS—isoleucine-glycine-serine
IGSM (O) SG—isoleucine-glycine-serine-methionine oxide-serine-glycine
Isoasp—isoaspartate
LCMS—liquid chromatography-coupled mass spectrometry
LDR—leucine-aspartic acid-arginine
LisoDR—leucine-isoaspartate-arginine
LOQ—limit of quantification
Met(O)—methionine sulfoxide
MS—mass spectrometry
MSG—methionine-serine-glycine
MW—molecular weight
MWCO—molecular weight cut-off
PBS—phosphate buffered saline
PS80—polysorbate 80
PVDF—polyvinylidene difluoride
RP-HPLC—reverse-phase high-performance liquid chromatography
SE-HPLC—size-exclusion high-performance liquid chromatography
TFA—trifluoroacetic acid
TFF—tangential flow filtration
UPLC—ultra-high-performance liquid chromatography

1. A liquid pharmaceutical formulation comprising a CNP compound, a buffering agent, an isotonicity agent, a preservative and optionally an antioxidant.

2. The liquid pharmaceutical formulation of claim 1 for storage for at least 6 months at 2 to 8° C.

3-4. (canceled)

5. The liquid pharmaceutical formulation of claim 1 for storage for 6 to 36 months at 5° C.

6. The liquid pharmaceutical formulation of claim 1, wherein the CNP compound is a CNP conjugate.

7. The liquid pharmaceutical formulation of claim 1, wherein the buffering agent is selected from the group consisting of acetic acid, succinic acid, citric acid, lactic acid, glutamic acid, fumaric acid, aspartic acid, glutaric acid, phosphoric acid, histidine, gluconic acid, tartaric acid, malic acid and Tris (tris (hydroxymethyl) aminomethane).

8. (canceled)

9. The liquid pharmaceutical formulation of claim 1, wherein the isotonicity agent is selected from the group consisting of mannitol, trehalose, sucrose, raffinose, gelatin, lactose, dibasic calcium phosphate, sorbitol, xylitol, glycine, histidine, ethanol, hydroxyethylstarch, potassium chloride, sodium chloride, dextrose, dextran, propylene glycol and glycerol.

10-12. (canceled)

13. The liquid pharmaceutical formulation of claim 1, wherein the preservative is selected from the group consisting of m-cresol, benzyl alcohol, benzoic acid, phenol, methylparaben, ethylparaben, propylparaben, butylparaben, potassium sorbate, chlorobutanol, benzyl alcohol, phenylmercuric nitrate, thimerosal, sorbic acid, potassium sorbate, chlorocresol, benzalkonium chloride, 2-ethoxyethanol, chlorhexidine, chlorobutanol, phenylethyl alcohol and phenylmercuric acetate.

14-16. (canceled)

17. The liquid pharmaceutical formulation of claim 1, wherein the pharmaceutical formulation comprises an antioxidant selected from the group consisting of methionine, butylhydroxytoluene, butylhydroxyanisol, tocopherol, propylgallate, ascorbic acid, sodium bisulfite, ethylenediaminetetraacetic acid (EDTA), cysteine, glutathione, monothioglycerol, poly(ethylenimine), vitamin E, ectoine and morin.

18. The liquid pharmaceutical formulation of claim 1, wherein the pH of the liquid formulation is from about pH 3.5 to about pH 5.5.

19. (canceled)

20. The liquid pharmaceutical formulation of claim 1, wherein the liquid formulation further comprises a pH-adjusting agent.

21. (canceled)

22. The liquid pharmaceutical formulation of claim 1, wherein the CNP compound is a reversible CNP conjugate that comprises a CNP moiety that is covalently and reversibly conjugated to a carrier moiety.

23. (canceled)

24. The liquid pharmaceutical formulation of claim 1, wherein the CNP compound is a CNP agonist.

25. The liquid pharmaceutical formulation of claim 1, wherein the CNP compound is a reversible CNP conjugate of formula (Ia): 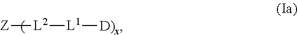
wherein
-D is a CNP moiety;
-L¹- is a reversible linker moiety;
-L²- is a single chemical bond or a spacer moiety;
-Z is a carrier moiety; and
x is an integer selected from the group consisting of 1, 2, 3, 4, 5, 6, 7, 8, 9, 10, 11, 12, 13, 14, 15 and 16.

26-28. (canceled)

29. The liquid pharmaceutical formulation of claim 6, wherein the liquid pharmaceutical formulation comprises:

| | | |
|---|---|---|
| CNP conjugate, of which CNP moiety | 0.1-20 | mg/ml |
| acetic acid | 0.3-3 | mg/ml |
| D-mannitol | 10-200 | mg/ml |
| m-cresol | 1-10 | mg/ml |

or wherein the liquid pharmaceutical formulation comprises:

| | | |
|---|---|---|
| CNP conjugate, of which CNP moiety | 0.1-20 | mg/ml, |
| acetic acid | 0.3-3 | mg/ml |
| trehalose dihydrate | 10-200 | mg/ml |
| m-cresol | 1-10 | mg/ml.. |

30. (canceled)

31. The liquid pharmaceutical formulation of claim 25, wherein the moiety -L¹- is of formula (II): 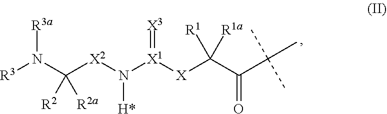
wherein the dashed line indicates the attachment to a nitrogen of -D which is a CNP moiety by forming an amide bond;
—X— is —C(R⁴R^{4a})—, —N(R⁴)—, —O—, —C(R⁴R^{4a})—C(R⁵R^{5a})—, —C(R⁵R^{5a})—C(R⁴R^{4a})—, —C(R⁴R^{4a})—N(R⁶)—, —N(R⁶)—C(R⁴R^{4a})—, —C(R⁴R^{4a})—O—, —O—C(R⁴R^{4a})— or —C(R⁷R^{7a})—;
X^{f} is C, or S (O);
—X²— is —C(R⁸R^{8a})— or —C(R⁸R^{8a})—C(R⁹R^{9a})—;
═X³ is ═O, ═S or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to the N atom to which they are attached through a sp³-hybridized carbon atom;
—R⁷ is —N(R¹⁰R^{10a}) or —NR¹⁰—(C═O)—R¹¹;
—R^{7a}, —R¹⁰, —R^{10a}, —R¹¹ are independently of each other —H; or C₁₋₆ alkyl;
optionally, one or more of the pairs —R^{1a}/—R^{4a}, —R^{1a}/—R^{5a}, —R^{1a}/—R^{7a}, —R^{4a}/—R^{5a}—R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁸/—R^{8a}, —R⁹/—R^{9a} are joined together with the atom to which they are attached to form a C₃₋₁₀ cycloalkyl; or 3- to 10-membered heterocyclyl;
optionally, one or more of the
pairs —R¹/—R⁴, —R¹/—R⁵, —R¹/—R⁶, —R¹/—R^{7a}, —R⁴/—R⁵, —R⁴/—R⁶, —R⁸/—R⁹, —R²/—R³ are joined together with the atoms to which they are attached to form a ring A;
optionally, —R³/—R^{3a} are joined together with the nitrogen atom to which they are attached to form a 3- to 10-membered heterocycle;
A is selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; and
wherein -L¹- is substituted with -L²-Z and wherein -L¹- is optionally further substituted, provided that the hydrogen marked with the asterisk in formula (II) is not replaced by -L²-Z or a substituent.

32. The liquid pharmaceutical formulation of claim 25, wherein the moiety -L¹-L²- is of formula (IId-ii): 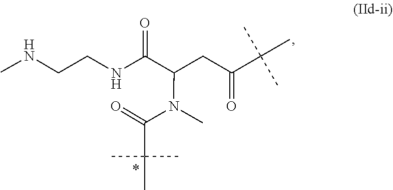
wherein
the unmarked dashed line indicates attachment to a nitrogen of an amino acid side chain of the ring moiety of -D by forming an amide bond; and the dashed line marked with the asterisk indicates attachment to —Z; wherein -D has the sequence of SEQ ID NO: 24 and -L¹- is conjugated to the lysine at position 26;
wherein —Z is of formula (h): 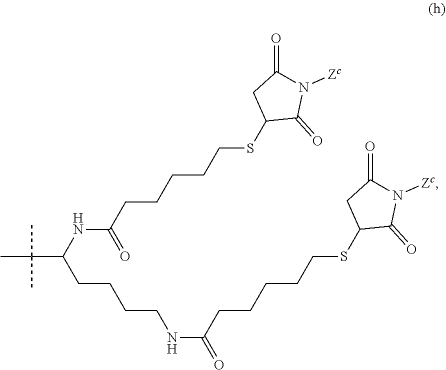
wherein
the dashed line indicates attachment to -L²-; and
each —Z^{c} is a moiety 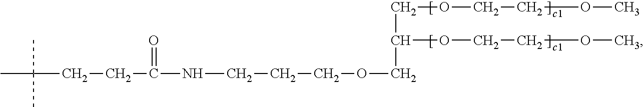
wherein
each c1 is an integer independently ranging from about 200 to 250.

33. A method of manufacturing the liquid pharmaceutical formulation of claim 1, wherein the method comprises the following steps:
(i) admixing the CNP compound with at least a buffering agent, an isotonicity agent, a preservative and optionally an antioxidant;
(ii) adjusting the pH of the admixture of step (i);
(iii) optionally, filtering the admixture from step (ii);
(iv) transferring amounts of the admixture from step (ii) or (iii) equivalent to the desired number of dosages into a container;
(v) sealing the container; and
wherein the order of steps (ii) and (iii) may optionally be reversed.

34-37. (canceled)

38. A container comprising the liquid pharmaceutical formulation of claim 1.

39-43. (canceled)

44. A method of treating, reducing the risk of or delaying in a human patient a disease treatable by CNP, the method comprising the step of administering to said patient of a therapeutically effective amount of the liquid pharmaceutical formulation of claim 1.

45. The method of claim 44, wherein the disease is selected from the group consisting of achondroplasia, hypochondroplasia, short stature, Noonan syndrome and SHOX deficiency.

46-47. (canceled)


SEQUENCE LISTING (106 sequences)

SEQ ID NO: 1 - protein, 22 aa, Homo sapiens
  DISULFID 6..22: note = intrachain disulfide bond
GLSKGCFGLKLDRIGSMSGLGC

SEQ ID NO: 2 - protein, 53 aa, Homo sapiens
  DISULFID 37..53: note = intrachain disulfide bond
DLRVDTKSRAAWARLLQEHPNARKYKGANKKGLSKGCFGLKLDRIGSMSGLGC

SEQ ID NO: 3 - protein, 54 aa, synthetic construct
  REGION 1..54: note = G-CNP-53
  DISULFID 38..54: note = intrachain disulfide bond
GDLRVDTKSRAAWARLLQEHPNARKYKGANKKGLSKGCFGLKLDRIGSMSGLGC

SEQ ID NO: 4 - protein, 54 aa, synthetic construct
  REGION 1..54: note = M-CNP-53
  DISULFID 38..54: note = intrachain disulfide bond
MDLRVDTKSRAAWARLLQEHPNARKYKGANKKGLSKGCFGLKLDRIGSMSGLGC

SEQ ID NO: 5 - protein, 54 aa, synthetic construct
  REGION 1..54: note = P-CNP-53
  DISULFID 38..54: note = intrachain disulfide bond
PDLRVDTKSRAAWARLLQEHPNARKYKGANKKGLSKGCFGLKLDRIGSMSGLGC

SEQ ID NO: 6 - protein, 53 aa, synthetic construct
  REGION 1..53: note = CNP-53 M48N
  DISULFID 37..53: note = intrachain disulfide bond
DLRVDTKSRAAWARLLQEHPNARKYKGANKKGLSKGCFGLKLDRIGSNSGLGC

SEQ ID NO: 7 - protein, 36 aa, synthetic construct
  REGION 1..36: note = CNP-53 with deletion of amino acids 15-31
  DISULFID 20..36: note = intrachain disulfide bond
DLRVDTKSRAAWARGLSKGCFGLKLDRIGSMSGLGC

SEQ ID NO: 8 - protein, 52 aa, synthetic construct
  REGION 1..52: note = CNP-52
  DISULFID 36..52: note = intrachain disulfide bond
LRVDTKSRAAWARLLQEHPNARKYKGANKKGLSKGCFGLKLDRIGSMSGLGC

SEQ ID NO: 9 - protein, 51 aa, synthetic construct
  REGION 1..51: note = CNP-51
  DISULFID 35..51: note = intrachain disulfide bond
RVDTKSRAAWARLLQEHPNARKYKGANKKGLSKGCFGLKLDRIGSMSGLGC

SEQ ID NO: 10 - protein, 50 aa, synthetic construct
  REGION 1..50: note = CNP-50
  DISULFID 34..50: note = intrachain disulfide bond
VDTKSRAAWARLLQEHPNARKYKGANKKGLSKGCFGLKLDRIGSMSGLGC

SEQ ID NO: 11 - protein, 49 aa, synthetic construct
  REGION 1..49: note = CNP-49
  DISULFID 33..49: note = intrachain disulfide bond
DTKSRAAWARLLQEHPNARKYKGANKKGLSKGCFGLKLDRIGSMSGLGC

SEQ ID NO: 12 - protein, 48 aa, synthetic construct
  REGION 1..48: note = CNP-48
  DISULFID 32..48: note = intrachain disulfide bond
TKSRAAWARLLQEHPNARKYKGANKKGLSKGCFGLKLDRIGSMSGLGC

SEQ ID NO: 13 - protein, 47 aa, synthetic construct
  REGION 1..47: note = CNP-47
  DISULFID 31..47: note = intrachain disulfide bond
KSRAAWARLLQEHPNARKYKGANKKGLSKGCFGLKLDRIGSMSGLGC

SEQ ID NO: 14 - protein, 46 aa, synthetic construct
  REGION 1..46: note = CNP-46
  DISULFID 30..46: note = intrachain disulfide bond
SRAAWARLLQEHPNARKYKGANKKGLSKGCFGLKLDRIGSMSGLGC

SEQ ID NO: 15 - protein, 45 aa, synthetic construct
  REGION 1..45: note = CNP-45
  DISULFID 29..45: note = intrachain disulfide bond
RAAWARLLQEHPNARKYKGANKKGLSKGCFGLKLDRIGSMSGLGC

SEQ ID NO: 16 - protein, 44 aa, synthetic construct
  REGION 1..44: note = CNP-44
  DISULFID 28..44: note = intrachain disulfide bond
AAWARLLQEHPNARKYKGANKKGLSKGCFGLKLDRIGSMSGLGC

SEQ ID NO: 17 - protein, 35 aa, synthetic construct
  REGION 1..35: note = CNP-44 with a deletion of amino acids 14-22
  DISULFID 19..35: note = intrachain disulfide bond
AAWARLLQEHPNAGLSKGCFGLKLDRIGSMSGLGC

SEQ ID NO: 18 - protein, 36 aa, synthetic construct
  REGION 1..36: note = CNP-44 with a deletion of amino acids 15-22
  DISULFID 20..36: note = intrachain disulfide bond
AAWARLLQEHPNARGLSKGCFGLKLDRIGSMSGLGC

SEQ ID NO: 19 - protein, 43 aa, synthetic construct
  REGION 1..43: note = CNP-43
  DISULFID 27..43: note = intrachain disulfide bond
AWARLLQEHPNARKYKGANKKGLSKGCFGLKLDRIGSMSGLGC

SEQ ID NO: 20 - protein, 42 aa, synthetic construct
  REGION 1..42: note = CNP-42
  DISULFID 26..42: note = intrachain disulfide bond
WARLLQEHPNARKYKGANKKGLSKGCFGLKLDRIGSMSGLGC

SEQ ID NO: 21 - protein, 41 aa, synthetic construct
  REGION 1..41: note = CNP-41
  DISULFID 25..41: note = intrachain disulfide bond
ARLLQEHPNARKYKGANKKGLSKGCFGLKLDRIGSMSGLGC

SEQ ID NO: 22 - protein, 40 aa, synthetic construct
  REGION 1..40: note = CNP-40
  DISULFID 24..40: note = intrachain disulfide bond
RLLQEHPNARKYKGANKKGLSKGCFGLKLDRIGSMSGLGC

SEQ ID NO: 23 - protein, 39 aa, synthetic construct
  REGION 1..39: note = CNP-39
  DISULFID 23..39: note = intrachain disulfide bond
LLQEHPNARKYKGANKKGLSKGCFGLKLDRIGSMSGLGC

SEQ ID NO: 24 - protein, 38 aa, synthetic construct
  REGION 1..38: note = CNP-38
  DISULFID 22..38: note = intrachain disulfide bond
LQEHPNARKYKGANKKGLSKGCFGLKLDRIGSMSGLGC

SEQ ID NO: 25 - protein, 37 aa, synthetic construct
  REGION 1..37: note = CNP-37
  DISULFID 21..37: note = intrachain disulfide bond
QEHPNARKYKGANKKGLSKGCFGLKLDRIGSMSGLGC

SEQ ID NO: 26 - protein, 37 aa, synthetic construct
  REGION 1..37: note = CNP-37 mit Q1pQ (pQ = pyroglutamate)
  SITE 1: note = MISC_FEATURE - X = pyroglutamate
  DISULFID 21..37: note = intrachain disulfide bond
XEHPNARKYKGANKKGLSKGCFGLKLDRIGSMSGLGC

SEQ ID NO: 27 - protein, 38 aa, synthetic construct
  REGION 1..38: note = G-CNP-37
  DISULFID 22..38: note = intrachain disulfide bond
GQEHPNARKYKGANKKGLSKGCFGLKLDRIGSMSGLGC

SEQ ID NO: 28 - protein, 38 aa, synthetic construct
  REGION 1..38: note = P-CNP-37
  DISULFID 22..38: note = intrachain disulfide bond
PQEHPNARKYKGANKKGLSKGCFGLKLDRIGSMSGLGC

SEQ ID NO: 29 - protein, 38 aa, synthetic construct
  REGION 1..38: note = M-CNP-37
  DISULFID 22..38: note = intrachain disulfide bond
MQEHPNARKYKGANKKGLSKGCFGLKLDRIGSMSGLGC

SEQ ID NO: 30 - protein, 39 aa, synthetic construct
  REGION 1..39: note = PG-CNP-37
  DISULFID 23..39: note = intrachain disulfide bond
PGQEHPNARKYKGANKKGLSKGCFGLKLDRIGSMSGLGC

SEQ ID NO: 31 - protein, 39 aa, synthetic construct
  REGION 1..39: note = MG-CNP-37
  DISULFID 23..39: note = intrachain disulfide bond
MGQEHPNARKYKGANKKGLSKGCFGLKLDRIGSMSGLGC

SEQ ID NO: 32 - protein, 37 aa, synthetic construct
  REGION 1..37: note = CNP-37 M32N
  DISULFID 21..37: note = intrachain disulfide bond
QEHPNARKYKGANKKGLSKGCFGLKLDRIGSNSGLGC

SEQ ID NO: 33 - protein, 38 aa, synthetic construct
  REGION 1..38: note = G-CNP-37 M32N
  DISULFID 22..38: note = intrachain disulfide bond
GQEHPNARKYKGANKKGLSKGCFGLKLDRIGSNSGLGC

SEQ ID NO: 34 - protein, 38 aa, synthetic construct
  REGION 1..38: note = G-CNP-37 K14Q
  DISULFID 22..38: note = intrachain disulfide bond
GQEHPNARKYKGANQKGLSKGCFGLKLDRIGSMSGLGC

SEQ ID NO: 35 - protein, 38 aa, synthetic construct
  REGION 1..38: note = G-CNP-37 K14P
  DISULFID 22..38: note = intrachain disulfide bond
GQEHPNARKYKGANPKGLSKGCFGLKLDRIGSMSGLGC

SEQ ID NO: 36 - protein, 37 aa, synthetic construct
  REGION 1..37: note = G-CNP-37 K14Q, deletion of amino acid 15
  DISULFID 21..37: note = intrachain disulfide bond
GQEHPNARKYKGANQGLSKGCFGLKLDRIGSMSGLGC

SEQ ID NO: 37 - protein, 38 aa, synthetic construct
  REGION 1..38: note = G-CNP-37 K14Q, K15Q
  DISULFID 22..38: note = intrachain disulfide bond
GQEHPNARKYKGANQQGLSKGCFGLKLDRIGSMSGLGC

SEQ ID NO: 38 - protein, 36 aa, synthetic construct
  REGION 1..36: note = CNP-36
  DISULFID 20..36: note = intrachain disulfide bond
EHPNARKYKGANKKGLSKGCFGLKLDRIGSMSGLGC

SEQ ID NO: 39 - protein, 35 aa, synthetic construct
  REGION 1..35: note = CNP-35
  DISULFID 19..35: note = intrachain disulfide bond
HPNARKYKGANKKGLSKGCFGLKLDRIGSMSGLGC

SEQ ID NO: 40 - protein, 34 aa, synthetic construct
  REGION 1..34: note = CNP-34
  DISULFID 18..34: note = intrachain disulfide bond
PNARKYKGANKKGLSKGCFGLKLDRIGSMSGLGC

SEQ ID NO: 41 - protein, 33 aa, synthetic construct
  REGION 1..33: note = CNP-33
  DISULFID 17..33: note = intrachain disulfide bond
NARKYKGANKKGLSKGCFGLKLDRIGSMSGLGC

SEQ ID NO: 42 - protein, 32 aa, synthetic construct
  REGION 1..32: note = CNP-32
  DISULFID 16..32: note = intrachain disulfide bond
ARKYKGANKKGLSKGCFGLKLDRIGSMSGLGC

SEQ ID NO: 43 - protein, 31 aa, synthetic construct
  REGION 1..31: note = CNP-31
  DISULFID 15..31: note = intrachain disulfide bond
RKYKGANKKGLSKGCFGLKLDRIGSMSGLGC

SEQ ID NO: 44 - protein, 30 aa, synthetic construct
  REGION 1..30: note = CNP-30
  DISULFID 14..30: note = intrachain disulfide bond
KYKGANKKGLSKGCFGLKLDRIGSMSGLGC

SEQ ID NO: 45 - protein, 29 aa, synthetic construct
  REGION 1..29: note = CNP-29
  DISULFID 13..29: note = intrachain disulfide bond
YKGANKKGLSKGCFGLKLDRIGSMSGLGC

SEQ ID NO: 46 - protein, 28 aa, synthetic construct
  REGION 1..28: note = CNP-28
  DISULFID 12..28: note = intrachain disulfide bond
KGANKKGLSKGCFGLKLDRIGSMSGLGC

SEQ ID NO: 47 - protein, 38 aa, synthetic construct
  REGION 1..38: note = GHKSEVAHRF-CNP-28
  DISULFID 22..38: note = intrachain disulfide bond
GHKSEVAHRFKGANKKGLSKGCFGLKLDRIGSMSGLGC

SEQ ID NO: 48 - protein, 27 aa, synthetic construct
  REGION 1..27: note = CNP-27
  DISULFID 11..27: note = intrachain disulfide bond
GANKKGLSKGCFGLKLDRIGSMSGLGC

SEQ ID NO: 49 - protein, 27 aa, synthetic construct
  REGION 1..27: note = CNP-27 K4Q, K5Q
  DISULFID 11..27: note = intrachain disulfide bond
GANQQGLSKGCFGLKLDRIGSMSGLGC

SEQ ID NO: 50 - protein, 27 aa, synthetic construct
  REGION 1..27: note = CNP-27 K4R, K5R
  DISULFID 11..27: note = intrachain disulfide bond
GANRRGLSKGCFGLKLDRIGSMSGLGC

SEQ ID NO: 51 - protein, 27 aa, synthetic construct
  DISULFID 11..27: note = intrachain disulfide bond
GANPRGLSKGCFGLKLDRIGSMSGLGC

SEQ ID NO: 52 - protein, 27 aa, synthetic construct
  REGION 1..27: note = CNP-27 K4S, K5S
  DISULFID 11..27: note = intrachain disulfide bond
GANSSGLSKGCFGLKLDRIGSMSGLGC

SEQ ID NO: 53 - protein, 30 aa, synthetic construct
  REGION 1..30: note = GAN-CNP-27 K4P, K5R
  DISULFID 14..30: note = intrachain disulfide bond
GANGANPRGLSRGCFGLKLDRIGSMSGLGC

SEQ ID NO: 54 - protein, 27 aa, synthetic construct
  REGION 1..27: note = CNP-27 K4R, K5R, K9R
  DISULFID 11..27: note = intrachain disulfide bond
GANRRGLSRGCFGLKLDRIGSMSGLGC

SEQ ID NO: 55 - protein, 27 aa, synthetic construct
  REGION 1..27: note = CNP-27 K4R, K5R, K9R, M22N
  DISULFID 11..27: note = intrachain disulfide bond
GANRRGLSRGCFGLKLDRIGSNSGLGC

SEQ ID NO: 56 - protein, 28 aa, synthetic construct
  REGION 1..28: note = P-CNP-27 K4R, K5R, K9R
  DISULFID 12..28: note = intrachain disulfide bond
PGANRRGLSRGCFGLKLDRIGSMSGLGC

SEQ ID NO: 57 - protein, 28 aa, synthetic construct
  REGION 1..28: note = M-CNP-27 K4R, K5R, K9R
  DISULFID 12..28: note = intrachain disulfide bond
MGANRRGLSRGCFGLKLDRIGSMSGLGC

SEQ ID NO: 58 - protein, 38 aa, synthetic construct; identical to SEQ ID NO: 47
  REGION 1..38: note = Human Serum Albumine Fragment - CNP-27
  DISULFID 22..38: note = intrachain disulfide bond

SEQ ID NO: 59 - protein, 38 aa, synthetic construct
  REGION 1..38: note = Human Serum Albumine Fragment - CNP-27 M22N
  DISULFID 22..38: note = intrachain disulfide bond
GHKSEVAHRFKGANKKGLSKGCFGLKLDRIGSNSGLGC

SEQ ID NO: 60 - protein, 39 aa, synthetic construct
  REGION 1..39: note = methionine - Human Serum Albumine Fragment - CNP-27
  DISULFID 23..39: note = intrachain disulfide bond
MGHKSEVAHRFKGANKKGLSKGCFGLKLDRIGSMSGLGC

SEQ ID NO: 61 - protein, 39 aa, synthetic construct
  REGION 1..39: note = proline - Human Serum Albumine Fragment - CNP-27
  DISULFID 23..39: note = intrachain disulfide bond
PGHKSEVAHRFKGANKKGLSKGCFGLKLDRIGSMSGLGC

SEQ ID NO: 62 - protein, 26 aa, synthetic construct
  REGION 1..26: note = CNP-26
  DISULFID 10..26: note = intrachain disulfide bond
ANKKGLSKGCFGLKLDRIGSMSGLGC

SEQ ID NO: 63 - protein, 25 aa, synthetic construct
  REGION 1..25: note = CNP-25
  DISULFID 9..25: note = intrachain disulfide bond
NKKGLSKGCFGLKLDRIGSMSGLGC

SEQ ID NO: 64 - protein, 24 aa, synthetic construct
  REGION 1..24: note = CNP-24
  DISULFID 8..24: note = intrachain disulfide bond
KKGLSKGCFGLKLDRIGSMSGLGC

SEQ ID NO: 65 - protein, 23 aa, synthetic construct
  REGION 1..23: note = CNP-23
  DISULFID 7..23: note = intrachain disulfide bond
KGLSKGCFGLKLDRIGSMSGLGC

SEQ ID NO: 66 - protein, 23 aa, synthetic construct
  REGION 1..23: note = R-CNP-22
  DISULFID 7..23: note = intrachain disulfide bond
RGLSKGCFGLKLDRIGSMSGLGC

SEQ ID NO: 67 - protein, 24 aa, synthetic construct
  REGION 1..24: note = ER-CNP-22
  DISULFID 8..24: note = intrachain disulfide bond
ERGLSKGCFGLKLDRIGSMSGLGC

SEQ ID NO: 68 - protein, 23 aa, synthetic construct
  REGION 1..23: note = R-CNP-22 K4R
  DISULFID 7..23: note = intrachain disulfide bond
RGLSRGCFGLKLDRIGSMSGLGC

SEQ ID NO: 69 - protein, 24 aa, synthetic construct
  REGION 1..24: note = ER-CNP-22 4KR
  DISULFID 8..24: note = intrachain disulfide bond
ERGLSRGCFGLKLDRIGSMSGLGC

SEQ ID NO: 70 - protein, 24 aa, synthetic construct
  REGION 1..24: note = RR-CNP-22
  DISULFID 8..24: note = intrachain disulfide bond
RRGLSRGCFGLKLDRIGSMSGLGC

SEQ ID NO: 71 - protein, 37 aa, synthetic construct
  REGION 1..37: note = HRGP fragment - CNP-22
  DISULFID 21..37: note = intrachain disulfide bond
GHHSHEQHPHGANQQGLSKGCFGLKLDRIGSMSGLGC

SEQ ID NO: 72 - protein, 38 aa, synthetic construct
  REGION 1..38: note = HRGP fragment - CNP-22
  DISULFID 22..38: note = intrachain disulfide bond
GAHHPHEHDTHGANQQGLSKGCFGLKLDRIGSMSGLGC

SEQ ID NO: 73 - protein, 37 aa, synthetic construct
  REGION 1..37: note = HRGP fragment - CNP-22
  DISULFID 21..37: note = intrachain disulfide bond
GHHSHEQHPHGANPRGLSKGCFGLKLDRIGSMSGLGC

SEQ ID NO: 74 - protein, 36 aa, synthetic construct
  REGION 1..36: note = IgG1(FC) fragment - CNP-22
  DISULFID 20..36: note = intrachain disulfide bond
GQPREPQVYTLPPSGLSKGCFGLKLDRIGSMSGLGC

SEQ ID NO: 75 - protein, 39 aa, synthetic construct
  REGION 1..39: note = Human Serum Albumine - CNP-22
  DISULFID 23..39: note = intrachain disulfide bond
GQHKDDNPNLPRGANPRGLSKGCFGLKLDRIGSMSGLGC

SEQ ID NO: 76 - protein, 37 aa, synthetic construct
  REGION 1..37: note = Human Serum Albumine - CNP-22
  DISULFID 21..37: note = intrachain disulfide bond
GERAFKAWAVARLSQGLSKGCFGLKLDRIGSMSGLGC

SEQ ID NO: 77 - protein, 35 aa, synthetic construct
  REGION 1..35: note = osteocrin NPR C inhibitor fragment - CNP-22
  DISULFID 19..35: note = intrachain disulfide bond
FGIPMDRIGRNPRGLSKGCFGLKLDRIGSMSGLGC

SEQ ID NO: 78 - protein, 40 aa, synthetic construct [sequence omitted]

SEQ ID NO: 79 - protein, 37 aa, synthetic construct
  REGION 1..37: note = IgG1(FC) fragment - CNP-22 K4R
  DISULFID 21..37: note = intrachain disulfide bond
GQPREPQVYTGANQQGLSRGCFGLKLDRIGSMSGLGC

SEQ ID NO: 80 - protein, 36 aa, synthetic construct
  REGION 1..36: note = Human Serum Albumine fragment - CNP-22 K4R
  DISULFID 20..36: note = intrachain disulfide bond
GVPQVSTSTGANQQGLSRGCFGLKLDRIGSMSGLGC

SEQ ID NO: 81 - protein, 37 aa, synthetic construct
  REGION 1..37: note = fibronectin fragment - CNP-22 K4R
  DISULFID 21..37: note = intrachain disulfide bond
GQPSSSSQSTGANQQGLSRGCFGLKLDRIGSMSGLGC

SEQ ID NO: 82 - protein, 37 aa, synthetic construct
  REGION 1..37: note = fibronectin fragment - CNP-22 K4R
  DISULFID 21..37: note = intrachain disulfide bond
GQTHSSGTQSGANQQGLSRGCFGLKLDRIGSMSGLGC

SEQ ID NO: 83 - protein, 37 aa, synthetic construct
  REGION 1..37: note = fibronectin fragment - CNP-22 K4R
  DISULFID 21..37: note = intrachain disulfide bond
GSTGQWHSESGANQQGLSRGCFGLKLDRIGSMSGLGC

SEQ ID NO: 84 - protein, 37 aa, synthetic construct
  REGION 1..37: note = zinc finger fragment - CNP-22 K4R
  DISULFID 21..37: note = intrachain disulfide bond
GSSSSSSSSSGANQQGLSRGCFGLKLDRIGSMSGLGC

SEQ ID NO: 85 - protein, 21 aa, synthetic construct
  REGION 1..21: note = CNP-21
  DISULFID 5..21: note = intrachain disulfide bond
LSKGCFGLKLDRIGSMSGLGC

SEQ ID NO: 86 - protein, 20 aa, synthetic construct
  REGION 1..20: note = CNP-20
  DISULFID 4..20: note = intrachain disulfide bond
SKGCFGLKLDRIGSMSGLGC

SEQ ID NO: 87 - protein, 19 aa, synthetic construct
  REGION 1..19: note = CNP-19
  DISULFID 3..19: note = intrachain disulfide bond
KGCFGLKLDRIGSMSGLGC

SEQ ID NO: 88 - protein, 18 aa, synthetic construct
  REGION 1..18: note = CNP-18
  DISULFID 2..18: note = intrachain disulfide bond
GCFGLKLDRIGSMSGLGC

SEQ ID NO: 89 - protein, 17 aa, synthetic construct
  REGION 1..17: note = CNP-17
  DISULFID 1..17: note = intrachain disulfide bond
CFGLKLDRIGSMSGLGC

SEQ ID NO: 90 - protein, 32 aa, synthetic construct
  REGION 1..32: note = BNP fragment - CNP-17 - BNP- fragment
  DISULFID 10..26: note = intrachain disulfide bond
SPKMVQGSGCFGLKLDRIGSMSGLGCKVLRRH

SEQ ID NO: 91 - protein, 38 aa, synthetic construct; identical to SEQ ID NO: 27
  REGION 1..38: note = CNP-38 L1G
  DISULFID 22..38: note = intrachain disulfide bond

SEQ ID NO: 92 - protein, 37 aa, synthetic construct; identical to SEQ ID NO: 25
  REGION 1..37: note = Ac-CNP-37
  MOD_RES 1: note = ACETYLATION
  DISULFID 21..37: note = intrachain disulfide bond

SEQ ID NO: 93 - protein, 37 aa, synthetic construct
  REGION 1..37: note = CNP-37, X = K or R, with the provision that at least one Xaa is R
  SITE 8: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  SITE 10: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  SITE 14: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  SITE 15: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  SITE 19: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  DISULFID 21..37: note = intrachain disulfide bond
  SITE 25: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
QEHPNARXYXGANXXGLSXGCFGLXLDRIGSMSGLGC

SEQ ID NO: 94 - protein, 37 aa, synthetic construct
  REGION 1..37: note = mutated CNP-37
  SITE 14: note = MISC_FEATURE - X is selected from the group consising of Lys, Arg, Pro, Ser and Gln, with the provision that at least one of the amino acids at position 14, 15, 19 and 25 is selected from the group consisting of Arg, Pro, Ser and Gln
  SITE 15: note = MISC_FEATURE - X is selected from the group consising of Lys, Arg, Pro, Ser and Gln, with the provision that at least one of the amino acids at position 14, 15, 19 and 25 is selected from the group consisting of Arg, Pro, Ser and Gln
  SITE 19: note = MISC_FEATURE - X is selected from the group consising of Lys, Arg, Pro, Ser and Gln, with the provision that at least one of the amino acids at position 14, 15, 19 and 25 is selected from the group consisting of Arg, Pro, Ser and Gln
  DISULFID 21..37: note = intrachain disulfide bond
  SITE 25: note = MISC_FEATURE - X is selected from the group consising of Lys, Arg, Pro, Ser and Gln, with the provision that at least one of the amino acids at position 14, 15, 19 and 25 is selected from the group consisting of Arg, Pro, Ser and Gln
QEHPNARKYKGANXXGLSXGCFGLXLDRIGSMSGLGC

SEQ ID NO: 95 - protein, 37 aa, synthetic construct
  REGION 1..37: note = mutated CNP-37
  REGION 14..15: note = MISC_FEATURE - X X is selected from the group consisting of Lys Arg, Arg Lys, Lys Pro, Pro Lys, Ser Ser, Arg Ser, Ser Arg, Gln Lys, Gln Arg, Lys Gln, Arg Gln, Arg Arg and Gln Gln
  DISULFID 21..37: note = intrachain disulfide bond
QEHPNARKYKGANXXGLSKGCFGLKLDRIGSMSGLGC

SEQ ID NO: 96 - protein, 15 aa, Homo sapiens
FGLKLDRIGSMSGLG

SEQ ID NO: 97 - protein, 15 aa, synthetic construct
FGLKLDRIGSNSGLG

SEQ ID NO: 98 - protein, 39 aa, Synthetic construct
  DISULFID 23..39: note = intrachain disulfide bond
PGQEHPQARRYRGAQRRGLSRGCFGLKLDRIGSMSGLGC

SEQ ID NO: 99 - protein, 39 aa, synthetic construct
  DISULFID 23..39: note = intrachain disulfide bond
PGQEHPNARRYRGANRRGLSRGCFGLKLDRIGSMSGLGC

SEQ ID NO: 100 - protein, 39 aa, synthetic construct
  DISULFID 23..39: note = intrachain disulfide bond
PGQEHPQARKYKGAQKKGLSKGCFGLKLDRIGSMSGLGC

SEQ ID NO: 101 - protein, 39 aa, synthetic construct; identical to SEQ ID NO: 98
  DISULFID 23..39: note = intrachain disulfide bond
  MOD_RES 1: note = acetylation

SEQ ID NO: 102 - protein, 39 aa, synthetic construct; identical to SEQ ID NO: 30
  DISULFID 23..39: note = intrachain disulfide bond
  MOD_RES 1: note = acetylation
  MOD_RES 39: note = C-terminal NH2

SEQ ID NO: 103 - protein, 39 aa, synthetic construct; identical to SEQ ID NO: 99
  DISULFID 23..39: note = intrachain disulfide bond
  MOD_RES 1: note = acetylation

SEQ ID NO: 104 - protein, 39 aa, synthetic construct; identical to SEQ ID NO: 99
  DISULFID 23..39: note = intrachain disulfide bond
  MOD_RES 1: note = acetylation
  MOD_RES 39: note = C-terminal NH2

SEQ ID NO: 105 - protein, 39 aa, synthetic construct; identical to SEQ ID NO: 98
  DISULFID 23..39: note = intrachain disulfide bond
  MOD_RES 1: note = acetylation
  MOD_RES 39: note = C-terminal NH2

SEQ ID NO: 106 - protein, 38 aa, Synthetic construct
  DISULFID 22..38: note = intrachain disulfide bond
LQEHPQARKYKGAQKKGLSKGCFGLKLDRIGSMSGLGC